(19) United States
(12) Patent Application Publication
(10) Pub. No.: US 2026/0226041 A1
(43) Pub. Date: Aug. 6, 2026

(54) ARYL HYDROCARBON RECEPTOR MODULATOR AND USE THEREOF

(71) Applicant: ARIGENX THERAPEUTICS CO., LTD., Shenzhen (CN)

(72) Inventor: Suoming ZHANG, Shenzhen (CN)

(73) Assignee: ARIGENX THERAPEUTICS CO., LTD., Shenzhen (CN)

(21) Appl. No.: 19/577,323

(22) Filed: Mar. 24, 2026

Related U.S. Application Data
(63) Continuation of application No. PCT/CN2024/118483, filed on Sep. 12, 2024.

(30) Foreign Application Priority Data
Sep. 28, 2023 (CN) 202311273015.0

(51) Int. Cl. C07D 417/14; C07D 491/056; C07F 9/6558

(52) U.S. Cl. CPC C07D 417/14; C07D 491/056; C07F 9/65586

(57) ABSTRACT

An aryl hydrocarbon receptor modulator is provided. The aryl hydrocarbon receptor modulator includes the following general formula (I):

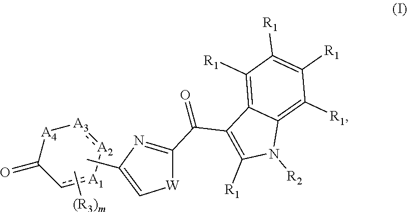

where W is C═C or S, A₁, A₂, A₃ are each independently a bond or C, and m is an integer from 1 to 7; R₃ is independently H, D, halogen, CN, OR, SR, N(R)R, —C(O)R, —C(O)OR, RC(O)O—, S(O)ₙR, unsubstituted C1-C8 alkyl or substituted C1-C8 alkyl with 1-6 Rₐ, unsubstituted C2-C8 alkenyl or substituted C2-C8 alkenyl with 1-6 Rₐ, unsubstituted C2-C8 alkynyl or substituted C2-C8 alkynyl with 1-6 Rₐ, or unsubstituted C3-C10 cycloalkyl or substituted C3-C10 cycloalkyl with 1-6 Rₐ; A₄ is independently O or NR₄; R₂ is H, —(CH₂O)ₐC(O)(CH₂)_{b}(O)_{c}(CH(R_{c}))_{d}(CH₂CH₂O)ₑR_{c}, —(CH₂O)ₐP(O)(Rₚ)₂, or —(CH₂O)ₐ(CH(R_{c}))_{d}(CH₂CH₂O)ₑR_{c}.

### CROSS-REFERENCE TO RELATED APPLICATIONS

This application is a continuation of International Application No. PCT/CN2024/118483, filed on Sep. 12, 2024, which claims priority to Chinese Patent Application No. 202311273015.0, filed on Sep. 28, 2023, the entire contents of each of which are hereby incorporated by reference.

### TECHNICAL FIELD

The present disclosure generally relates to the technical field of pharmaceutical synthesis, and in particular, to an aryl hydrocarbon receptor modulator and a use thereof.

### BACKGROUND

The aryl hydrocarbon receptor (AHR) is a member of the bHLH-PER-ARNT-SIM (bHLH-PAS) subfamily within the basic helix-loop-helix (bHLH) superfamily. The AHR is the only ligand-activatable receptor in the bHLH-PAS family [Murray et al., Nat. Rev. Cancer, 2014, 14(12), 801-814; Bersten et al., Nat. Rev. Cancer, 2013, 13(12), 827-841].

Agonists of the AHR are highly diverse, including traditionally recognized aromatic hydrocarbon xenobiotics, such as 2,3,7,8-tetrachlorodibenzo-p-dioxin (TCDD). Over the past two decades, numerous endogenous agonists have also been gradually identified, such as 2-(1′H-indole-3′-carbonyl)-thiazole-4-carboxylic acid methyl ester (ITE) and the tryptophan metabolite kynurenic acid. The agonists of the AHR can also be derived from dietary intake, such as indole-3-carbinol (I3C) from cruciferous vegetables, and metabolites (e.g., indoleacetic acid (IAA)) from the gut microbiota. After activation by the agonist, the AHR translocate from the cytoplasm into the nucleus and forms a heterodimer with the aryl hydrocarbon receptor nuclear translocator (ARNT). The heterodimer binds to the xenobiotic response element (XRE) in DNA, thereby initiating transcription of XRE-regulated genes (e.g., genes encoding cytochrome CYP450 enzymes (e.g., CYP1A1)). The AHR can also regulate XRE-independent gene expression and metabolism through protein-protein interactions, for example, with the estrogen receptor.

The AHR pathway regulates numerous key innate and adaptive immune responses. Studies have found that some AHR agonists promote T-helper 17 (Th17) cell differentiation and interleukin-17 (IL-17) secretion. In contrast, other AHR agonists can induce the lateral differentiation of Th17 cells into regulatory T (Treg) cells and enhance the suppressive activity of Treg cells [Quintana et al., Nature, 2008, 453(7191), 65-71; Mezrich et al., J. Immunol., 2010, 185(6), 3190-3198]. Studies have demonstrated that AHR activation can inhibit macrophage-regulated innate inflammatory responses (e.g., reducing lipopolysaccharide (LPS)-induced expression of IL-1b, IL-6, IL-12, and TNFalpha) and inhibit dendritic cells (reducing dendritic cell activation and promoting IL-10 expression) [Kimura et al., J. Exp. Med., 2009, 206(9), 2027-2035; Wang et al., Clin. Exp. Immunol., 2014, 177(2), 521-530; Wei et al., Lab. Invest., 2014, 94(5), 528-535; Nguyen et al., Proc. Natl. Acad. Sci. USA, 2010, 107(46), 19961-19966].

As an important key factor in immune regulation, the AHR is considered a key target for immune-related diseases. Studies have indicated that the AHR plays a regulatory role in models of septic shock, herpes simplex virus ocular infection, and Toxoplasma gondii infection. The agonists of the AHR also exhibit protective effects in immune diseases such as multiple sclerosis, inflammatory bowel disease, rheumatoid arthritis, and psoriasis. The immune-regulatory activity associated with the AHR also plays an important role in numerous inflammation-related metabolic diseases, such as atherosclerosis, type I diabetes, chronic kidney disease, bone metabolism-related diseases, and pulmonary arterial hypertension. Therefore, it is of great significance to develop novel agonists targeting the AHR for providing new drugs for the treatment of immune-related diseases.

### SUMMARY

Some embodiments of the present disclosure provide an aryl hydrocarbon receptor modulator or an enantiomer, a prodrug, or a pharmaceutically acceptable salt thereof. The aryl hydrocarbon receptor modulator includes the following general formula (I):

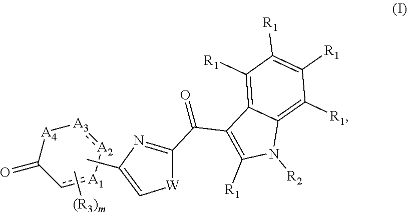

wherein
W is C═C or S;
A₁, A₂, and A₃ are each independently a bond or C; m is an integer from 1 to 7; R₃ is independently H, D, halogen, CN, OR, SR, N(R)R, —C(O)R, —C(O)OR, RC(O)O—, S(O)ₙR, unsubstituted C1-C8 alkyl or substituted C1-C8 alkyl with 1-6 Rₐ, unsubstituted C2-C8 alkenyl or substituted C2-C8 alkenyl with 1-6 Rₐ, unsubstituted C2-C8 alkynyl or substituted C2-C8 alkynyl with 1-6 Rₐ, or unsubstituted C3-C10 cycloalkyl or substituted C3-C10 cycloalkyl with 1-6 Rₐ; and Rₐ is D, halogen, OR, SR, N(R)R, —C(O)R, —C(O)OR, RC(O)O—, or S(O)ₙR;
A₄ is independently O or NR₄; R₄ is H, unsubstituted C1-C8 alkyl or substituted C1-C8 alkyl with 1-6 R_{b}, unsubstituted C2-C8 alkenyl or substituted C2-C8 alkenyl with 1-6 R_{b}, unsubstituted C2-C8 alkynyl or substituted C2-C8 alkynyl with 1-6 R_{b}, or unsubstituted C3-C10 cycloalkyl or substituted C3-C10 cycloalkyl with 1-6 Rb; and R_{b} is D, halogen, OR, SR, N(R)R, —C(O)R, —C(O)OR, RC(O)O—, or S(O)ₙR;
R₁ is independently H, D, halogen, CN, OR, SR, N(R)R, —C(O)R, —C(O)OR, RC(O)O—, S(O)ₙR, unsubstituted C1-C8 alkyl or substituted C1-C8 alkyl with 1-6 R_{b}, unsubstituted C2-C8 alkenyl or substituted C2-C8 alkenyl with 1-6 R_{b}, unsubstituted C2-C8 alkynyl or substituted C2-C8 alkynyl with 1-6 R_{b}, or unsubstituted C3-C10 cycloalkyl or substituted C3-C10 cycloalkyl with 1-6 R_{b}; Rb is D, halogen, OR, SR, N(R)R, —C(O)R, —C(O)OR, RC(O)O—, or S(O)ₙR; or two adjacent or meta R1 groups form a 3-10 membered carbocycle or a 3-10 membered heterocycle containing 1 to 3 heteroatoms selected from N, O, or S;
   n is 1 or 2;
   R is independently H, C1-C8 alkyl, C2-C8 alkenyl, C2-C8 alkynyl, or C3-C10 cycloalkyl; and
   R₂ is H, —(CH₂O)ₐC(O)(CH₂)_{b}(O)_{c}(CH(R_{c}))_{d}(CH₂CH₂O)ₑR_{c}, —(CH₂O)ₐP(O)(Rₚ)₂, or —(CH₂O)ₐ(CH(R_{c}))_{d}(CH₂CH₂O)ₑR_{c}, Rₚ is independently selected form (CH₂)_{b}(O)_{c}(CH(R_{c}))_{d}(CH₂CH₂O)ₑR_{c}; R_{c} is independently H, D, C1-C8 alkyl, NH₂—, (C1-C8 alkyl)NH—, (C1-C8 alkyl)₂N—, NH₂(C1-C8 alkyl)-, OH, HO—(C1-C8 alkyl)-, or (C1-C8 alkyl)O—; a, b, c, and d are each independently 0 or 1; and e is an integer from 0 to 3.

In some embodiments, when W is S, the general formula (I) is a general formula (Ia):

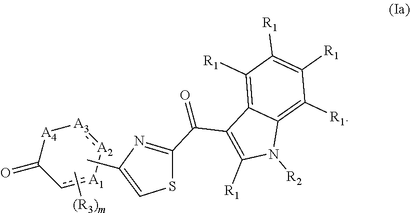

In some embodiments, when A₄ is 0, the general formula (Ia) is a general formula (Ia1):

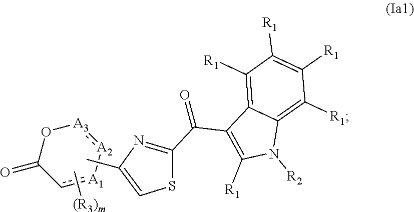

or
when A₄ is NR₄, the general formula (Ia) is a general formula (Ia2):

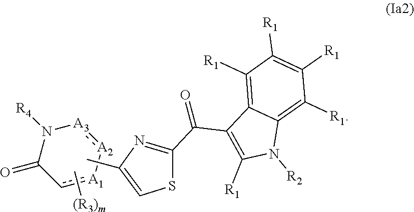

In some embodiments, when W is C═C, the general formula (I) is a general formula (Ib):

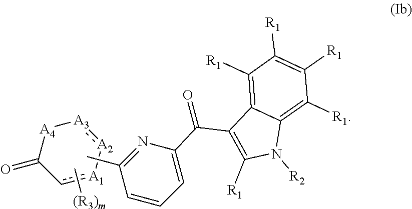

In some embodiments, when A₄ is O, the general formula (Ib) is a general formula (Ib1):

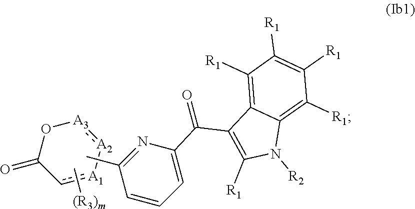

or
when A₄ is NR₄, the general formula (Ib) is a general formula (Ib2):

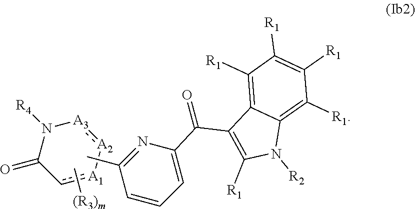

In some embodiments, A₁, A₂, and A₃ are each independently a bond or C; m is an integer from 1 to 5; R₃ is independently H, D, halogen, CN, OR, SR, N(R)R, —C(O)R, —C(O)OR, RC(O)O—, S(O)ₙR, unsubstituted C1-C6 alkyl or substituted C1-C6 alkyl with 1-3 Rₐ, unsubstituted C2-C6 alkenyl OR substituted C2-C6 alkenyl with 1-3 Rₐ, unsubstituted C2-C6 alkynyl or substituted C2-C6 alkynyl with 1-3 Rₐ, or unsubstituted C3-C8 cycloalkyl or substituted C3-C8 cycloalkyl with 1-3 Rₐ; and Rₐ is D, halogen, OR, SR, N(R)R, —C(O)R, —C(O)OR, RC(O)O—, or S(O)ₙR; n is 1 or 2; and
R is independently H, C1-C6 alkyl, C2-C6 alkenyl, C2-C6 alkynyl, or C3-C8 cycloalkyl.

In some embodiments, A₁, A₂, and A₃ are each independently a bond or C; m is an integer from 1 to 3; R₃ is independently H, D, halogen, CN, OR, SR, N(R)R, —C(O)R, —C(O)OR, RC(O)O—, S(O)ₙR, unsubstituted C1-C4 alkyl or substituted C1-C4 alkyl with 1-2 Rₐ, unsubstituted C2-C4 alkenyl or substituted C2-C4 alkenyl with 1-2 Rₐ, unsubstituted C2-C4 alkynyl or substituted C2-C4 alkynyl with 1-2 Rₐ, or unsubstituted C3-C7 cycloalkyl or substituted C3-C7 cycloalkyl with 1-2 Rₐ; and Rₐ is D, halogen, OR, SR, N(R)R, —C(O)R, —C(O)OR, RC(O)O—, or S(O)ₙR; n is 1 or 2; and
R is independently H, C1-C4 alkyl, C2-C4 alkenyl, C2-C4 alkynyl, or C3-C7 cycloalkyl.

In some embodiments, A₁, A₂, and A₃ are each independently a bond or C; m is an integer from 1 to 2; R₃ is independently H, D, halogen, CN, OR, SR, N(R)R, —C(O)R, —C(O)OR, RC(O)O—, S(O)ₙR, unsubstituted C1-C3 alkyl or substituted C1-C3 alkyl with 1 Rₐ, unsubstituted C2-C3 alkenyl or substituted C2-C3 alkenyl with 1 Rₐ, unsubstituted C2-C3 alkynyl or substituted C2-C3 alkynyl with 1 Rₐ, or unsubstituted C5-C6 cycloalkyl or substituted C5-C6 cycloalkyl with 1 Rₐ; and Rₐ is D, halogen, OR, SR, N(R)R, —C(O)R, —C(O)OR, RC(O)O—, or S(O)ₙR; n is 1 or 2; and R is independently H, C1-C3 alkyl, C2-C3 alkenyl, C2-C3 alkynyl, or C5-C6 cycloalkyl.

In some embodiments, R₁ is independently H, D, halogen, CN, OR, SR, N(R)R, —C(O)R, —C(O)OR, RC(O)O—, S(O)ₙR, unsubstituted C1-C6 alkyl or substituted C1-C6 alkyl with 1-3 R_{b}, unsubstituted C2-C6 alkenyl or substituted C2-C6 alkenyl with 1-3 R_{b}, unsubstituted C2-C6 alkynyl or substituted C2-C6 alkynyl with 1-3 R_{b}, or unsubstituted C3-C8 cycloalkyl or substituted C3-C8 cycloalkyl with 1-3 R_{b}; R_{b} is D, halogen, OR, SR, N(R)R, —C(O)R, —C(O)OR, RC(O)O—, or S(O)ₙR; or two adjacent or meta R₁ groups form a 3-8 membered carbocycle or a 3-8 membered heterocycle containing 1-3 heteroatoms selected from N, O, or S; n is 1 or 2; and R is independently H, C1-C6 alkyl, C2-C6 alkenyl, C2-C6 alkynyl, or C3-C8 cycloalkyl.

In some embodiments, R₁ is independently H, D, halogen, CN, OR, SR, N(R)R, —C(O)R, —C(O)OR, RC(O)O—, S(O)ₙR, unsubstituted C1-C4 alkyl or substituted C1-C4 alkyl with 1-2 R_{b}, unsubstituted C2-C4 alkenyl or substituted C2-C4 alkenyl with 1-2 R_{b}, unsubstituted C2-C4 alkynyl or substituted C2-C4 alkynyl with 1-2 R_{b}, or unsubstituted C3-C7 cycloalkyl or substituted C3-C7 cycloalkyl with 1-2 R_{b}; R_{b} is D, halogen, OR, SR, N(R)R, —C(O)R, —C(O)OR, RC(O)O—, or S(O)ₙR; or two adjacent or meta R₁ groups form a 3-7 membered carbocycle or a 3-7 membered heterocycle containing 1-2 heteroatoms selected from N, O, or S; n is 1 or 2; and R is independently H, C1-C4 alkyl, C2-C4 alkenyl, C2-C4 alkynyl, or C3-C7 cycloalkyl.

In some embodiments, R₁ is independently H, D, halogen, CN, OR, SR, N(R)R, —C(O)R, —C(O)OR, RC(O)O—, S(O)ₙR, unsubstituted C1-C3 alkyl or substituted C1-C3 alkyl with 1 R_{b}, unsubstituted C2-C3 alkenyl or substituted C2-C3 alkenyl with 1 R_{b}, unsubstituted C2-C3 alkynyl or substituted C2-C3 alkynyl with 1 R_{b}, or unsubstituted C5-C6 cycloalkyl or substituted C5-C6 cycloalkyl with 1 R_{b}; R_{b} is D, halogen, OR, SR, N(R)R, —C(O)R, —C(O)OR, RC(O)O—, or S(O)ₙR; or two adjacent or meta R₁ groups form a 5-6 membered carbocycle or a 5-6 membered heterocycle containing 1-2 heteroatoms selected from N, O, or S; n is 1 or 2; and R is independently H, C1-C3 alkyl, C2-C3 alkenyl, C2-C3 alkynyl, or C5-C6 cycloalkyl.

In some embodiments, R₂ is H, —(CH₂O)ₐC(O)(CH₂)_{b}(O)_{c}(CH(R_{c}))_{d}(CH₂CH₂O)ₑR_{c}, —(CH₂O)ₐP(O)(Rₚ)₂, or —(CH₂O)ₐ(CH(R_{c}))_{d}(CH₂CH₂O)ₑR_{c}, Rₚ is independently selected from (CH₂)_{b}(O)_{c}(CH(R_{c}))_{d}(CH₂CH₂O)ₑR_{c}; R_{c} is independently H, D, C1-C6 alkyl, NH₂—, (C1-C6 alkyl)NH—, (C1-C6 alkyl)₂N—, NH₂(C1-C6 alkyl)-, OH, HO—(C1-C6 alkyl)-, or (C1-C6 alkyl)O—; a, b, c, and d are each independently 0 or 1; and e is an integer from 0 to 2.

In some embodiments, R₂ is H, —(CH₂O)ₐC(O)(CH₂)_{b}(O)_{c}(CH(R_{c}))_{d}(CH₂CH₂O)ₑR_{c}, —(CH₂O)ₐP(O)(Rₚ)₂, or —(CH₂O)ₐ(CH(R_{c}))_{d}(CH₂CH₂O)ₑR_{c}; Rₚ is independently selected from (CH₂)_{b}(O)_{c}(CH(R_{c}))_{d}(CH₂CH₂O)ₑR_{c}; R_{c} is independently H, D, C1-C4 alkyl, NH₂—, (C1-C4 alkyl)NH—, (C1-C4 alkyl)₂N—, NH₂(C1-C4)alkyl-, OH, HO—(C1-C4)alkyl-, or (C1-C4 alkyl)O—; a, b, c, and d are each independently 0 or 1; and e is an integer of 0 or 1.

In some embodiments, R₂ is H, —(CH₂O)ₐC(O)(CH₂)_{b}(O)_{c}(CH(R_{c}))_{d}(CH₂CH₂O)ₑR_{c}, —(CH₂O)ₐP(O)(Rₚ)₂, or —(CH₂O)ₐ(CH(R_{c}))_{d}(CH₂CH₂O)ₑR_{c}; Rₚ is independently selected from (CH₂)_{b}(O)_{c}(CH(R_{c}))_{d}(CH₂CH₂O)ₑR_{c}; R_{c} is independently H, D, C1-C3 alkyl, NH₂—, (C1-C3 alkyl)NH—, (C1-C3 alkyl)₂N—, NH₂(C1-C3)alkyl-, OH, HO—(C1-C3)alkyl-, or (C1-C3 alkyl)O—; a, b, c, and d are each independently 0 or 1; and e is an integer of 0 or 1.

In some embodiments, R₄ is H, unsubstituted C1-C6 alkyl or substituted C1-C6 alkyl with 1-3 R_{b}, unsubstituted C2-C6 alkenyl or substituted C2-C6 alkenyl with 1-3 R_{b}, unsubstituted C2-C6 alkynyl or substituted C2-C6 alkynyl with 1-3 R_{b}, or unsubstituted C3-C8 cycloalkyl or substituted C3-C8 cycloalkyl with 1-3 R_{b}; and R_{b} is D, halogen, OR, SR, N(R)R, —C(O)R, —C(O)OR, RC(O)O—, or S(O)ₙR; and R is independently H, D, C1-C6 alkyl, C2-C6 alkenyl, C2-C6 alkynyl, or C3-C8 cycloalkyl.

In some embodiments, R₄ is H, unsubstituted C1-C4 alkyl or substituted C1-C4 alkyl with 1-2 R_{b}, unsubstituted C2-C4 alkenyl or substituted C2-C4 alkenyl with 1-2 R_{b}, unsubstituted C2-C4 alkynyl or substituted C2-C4 alkynyl with 1-2 R_{b}, or unsubstituted C3-C7 cycloalkyl or substituted C3-C7 cycloalkyl with 1-2 R_{b}; and R_{b} is D, halogen, OR, SR, N(R)R, —C(O)R, —C(O)OR, RC(O)O—, or S(O)ₙR; and R is independently H, D, C1-C4 alkyl, C2-C4 alkenyl, C2-C4 alkynyl, or C3-C7 cycloalkyl.

In some embodiments, R₄ is H, unsubstituted C1-C3 alkyl or substituted C1-C3 alkyl with 1 R_{b}, unsubstituted C2-C3 alkenyl or substituted C2-C3 alkenyl with 1 R_{b}, unsubstituted C2-C3 alkynyl or substituted C2-C3 alkynyl with 1 R_{b}, or unsubstituted C5-C6 cycloalkyl or substituted C5-C6 cycloalkyl with 1 R_{b}; and R_{b} is D, halogen, OR, SR, N(R)R, —C(O)R, —C(O)OR, RC(O)O—, or S(O)ₙR; and R is independently H, D, C1-C3 alkyl, C2-C3 alkenyl, C2-C3 alkynyl, or C5-C6 cycloalkyl.

In some embodiments, the aryl hydrocarbon receptor modulator is one of the following compounds:

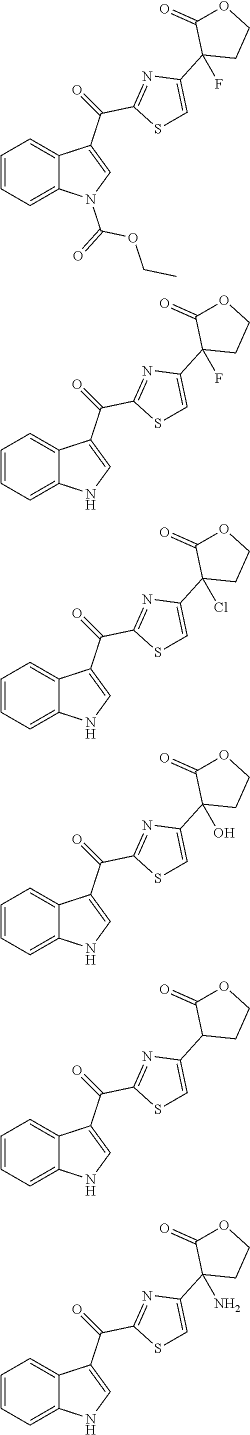

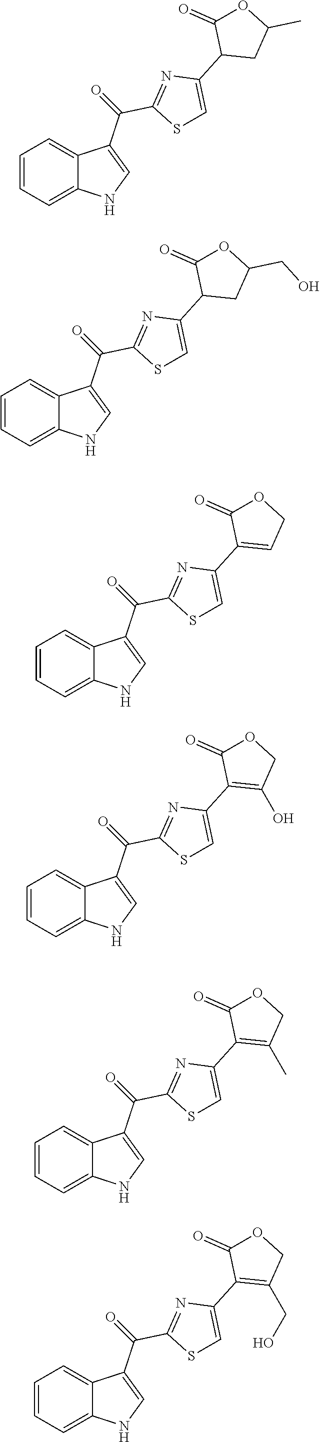 [SMILES: CC1CC(c2csc(C(=O)c3c[nH]c4ccccc34)n2)C(=O)O1 ; O=C(c1nc(C2CC(CO)OC2=O)cs1)c1c[nH]c2ccccc12 ; O=C1OCC=C1c1csc(C(=O)c2c[nH]c3ccccc23)n1 ; O=C1OCC(O)=C1c1csc(C(=O)c2c[nH]c3ccccc23)n1 ; CC1=C(c2csc(C(=O)c3c[nH]c4ccccc34)n2)C(=O)OC1 ; O=C1OCC(CO)=C1c1csc(C(=O)c2c[nH]c3ccccc23)n1]

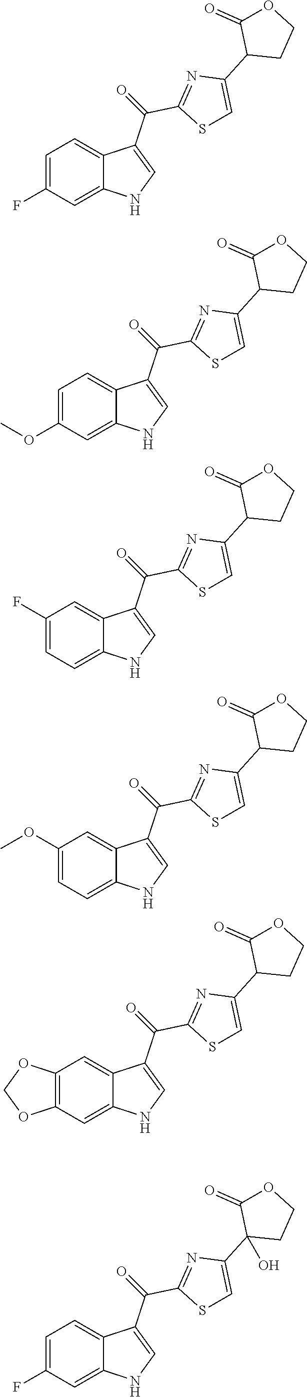 [SMILES: O=C(c1nc(C2CCOC2=O)cs1)c1c[nH]c2cc(F)ccc12 ; COc1ccc2c(C(=O)c3nc(C4CCOC4=O)cs3)c[nH]c2c1 ; O=C(c1nc(C2CCOC2=O)cs1)c1c[nH]c2ccc(F)cc12 ; COc1ccc2[nH]cc(C(=O)c3nc(C4CCOC4=O)cs3)c2c1 ; O=C(c1nc(C2CCOC2=O)cs1)c1c[nH]c2cc3c(cc12)OCO3 ; O=C(c1nc(C2(O)CCOC2=O)cs1)c1c[nH]c2cc(F)ccc12]

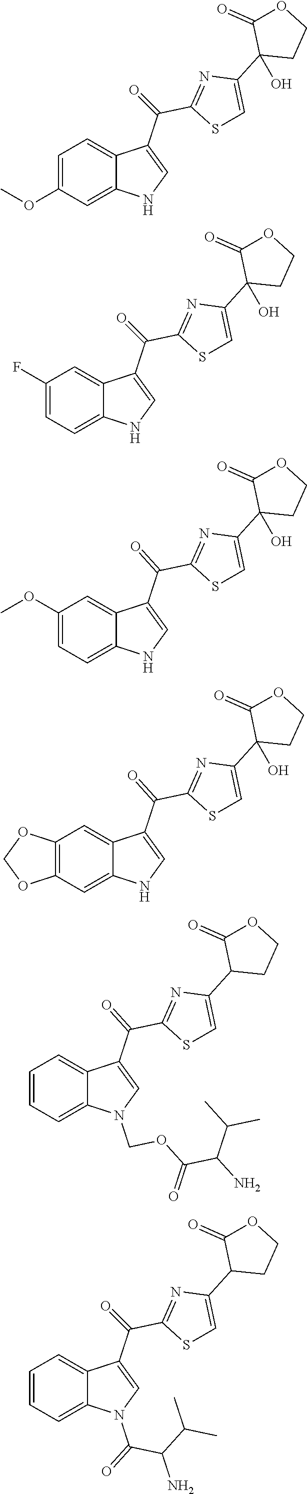

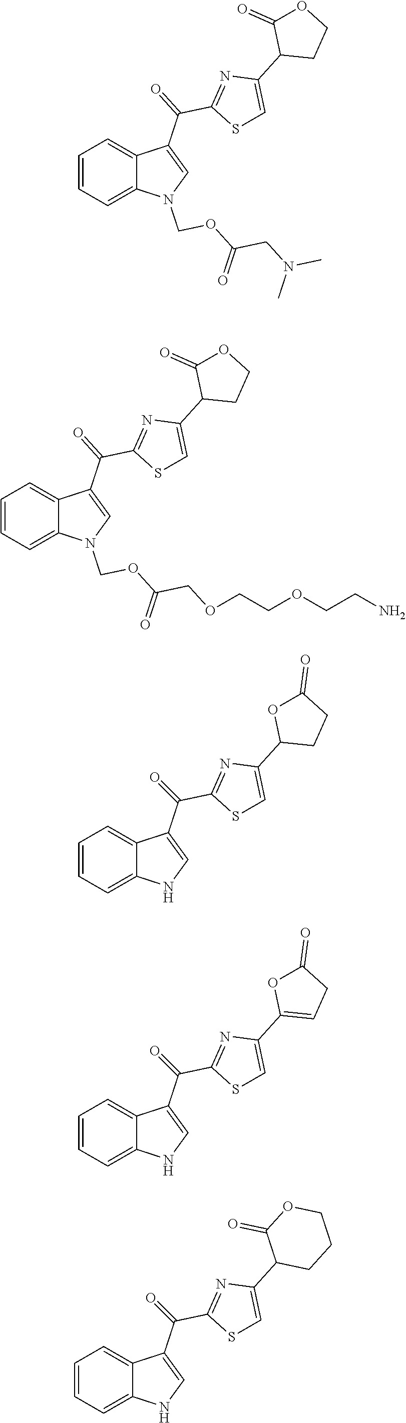 [SMILES: CN(C)CC(=O)OCn1cc(C(=O)c2nc(C3CCOC3=O)cs2)c2ccccc21 ; NCCOCCOCC(=O)OCn1cc(C(=O)c2nc(C3CCOC3=O)cs2)c2ccccc21 ; O=C1CCC(c2csc(C(=O)c3c[nH]c4ccccc34)n2)O1 ; O=C1CC=C(c2csc(C(=O)c3c[nH]c4ccccc34)n2)O1 ; O=C(c1nc(C2CCCOC2=O)cs1)c1c[nH]c2ccccc12]

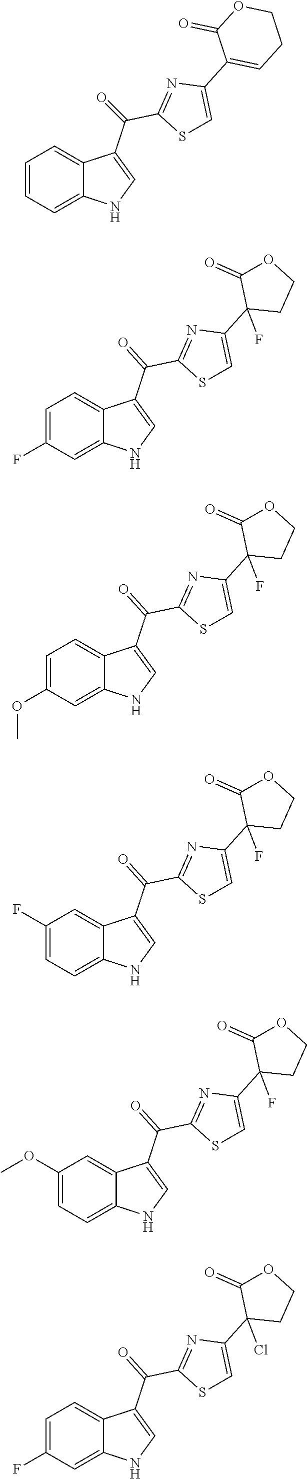 [SMILES: O=C1OCCC=C1c1csc(C(=O)c2c[nH]c3ccccc23)n1 ; O=C(c1nc(C2(F)CCOC2=O)cs1)c1c[nH]c2cc(F)ccc12 ; COc1ccc2c(C(=O)c3nc(C4(F)CCOC4=O)cs3)c[nH]c2c1 ; O=C(c1nc(C2(F)CCOC2=O)cs1)c1c[nH]c2ccc(F)cc12 ; COc1ccc2[nH]cc(C(=O)c3nc(C4(F)CCOC4=O)cs3)c2c1 ; O=C(c1nc(C2(Cl)CCOC2=O)cs1)c1c[nH]c2cc(F)ccc12]

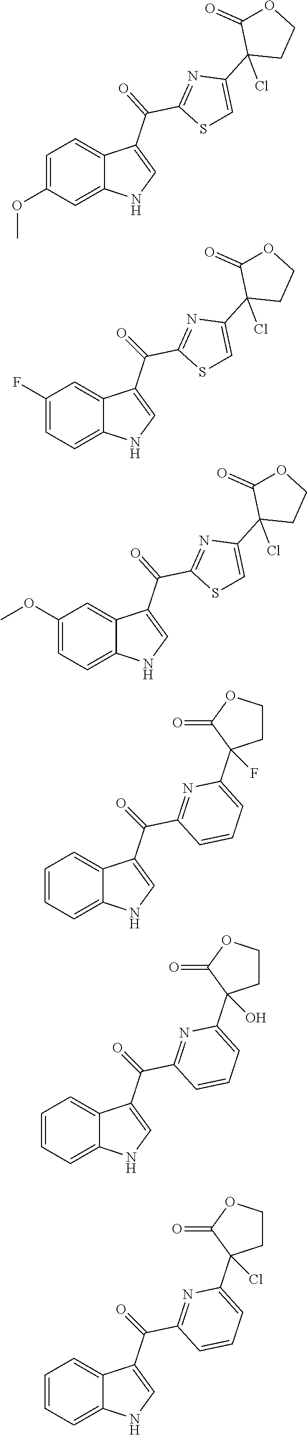 [SMILES: COc1ccc2c(C(=O)c3nc(C4(Cl)CCOC4=O)cs3)c[nH]c2c1 ; O=C(c1nc(C2(Cl)CCOC2=O)cs1)c1c[nH]c2ccc(F)cc12 ; COc1ccc2[nH]cc(C(=O)c3nc(C4(Cl)CCOC4=O)cs3)c2c1 ; O=C(c1cccc(C2(F)CCOC2=O)n1)c1c[nH]c2ccccc12 ; O=C(c1cccc(C2(O)CCOC2=O)n1)c1c[nH]c2ccccc12 ; O=C(c1cccc(C2(Cl)CCOC2=O)n1)c1c[nH]c2ccccc12]

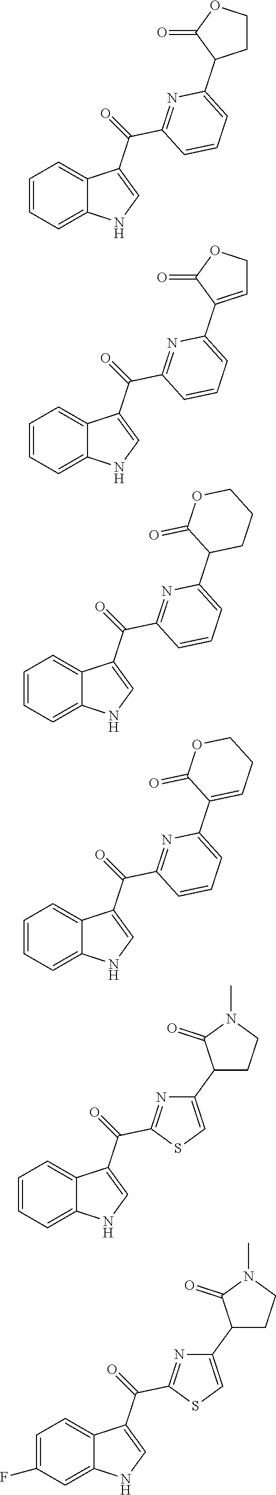 [SMILES: O=C(c1cccc(C2CCOC2=O)n1)c1c[nH]c2ccccc12 ; O=C1OCC=C1c1cccc(C(=O)c2c[nH]c3ccccc23)n1 ; O=C(c1cccc(C2CCCOC2=O)n1)c1c[nH]c2ccccc12 ; O=C1OCCC=C1c1cccc(C(=O)c2c[nH]c3ccccc23)n1 ; CN1CCC(c2csc(C(=O)c3c[nH]c4ccccc34)n2)C1=O ; CN1CCC(c2csc(C(=O)c3c[nH]c4cc(F)ccc34)n2)C1=O]

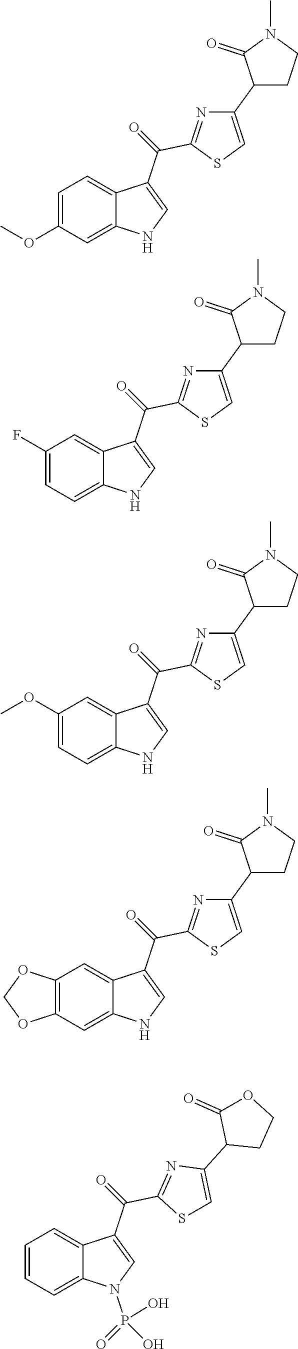 [SMILES: COc1ccc2c(C(=O)c3nc(C4CCN(C)C4=O)cs3)c[nH]c2c1 ; CN1CCC(c2csc(C(=O)c3c[nH]c4ccc(F)cc34)n2)C1=O ; COc1ccc2[nH]cc(C(=O)c3nc(C4CCN(C)C4=O)cs3)c2c1 ; CN1CCC(c2csc(C(=O)c3c[nH]c4cc5c(cc34)OCO5)n2)C1=O ; O=C(c1nc(C2CCOC2=O)cs1)c1cn(P(=O)(O)O)c2ccccc12]

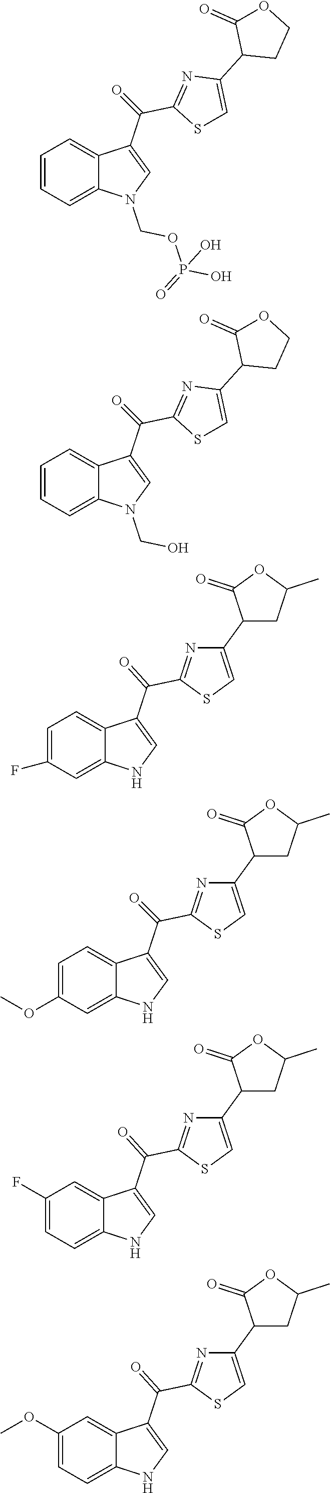

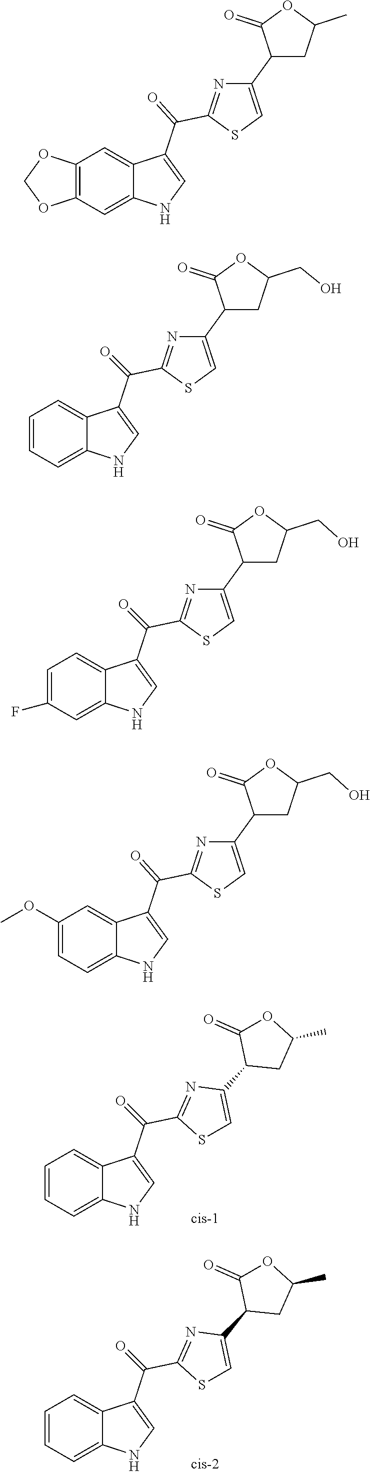

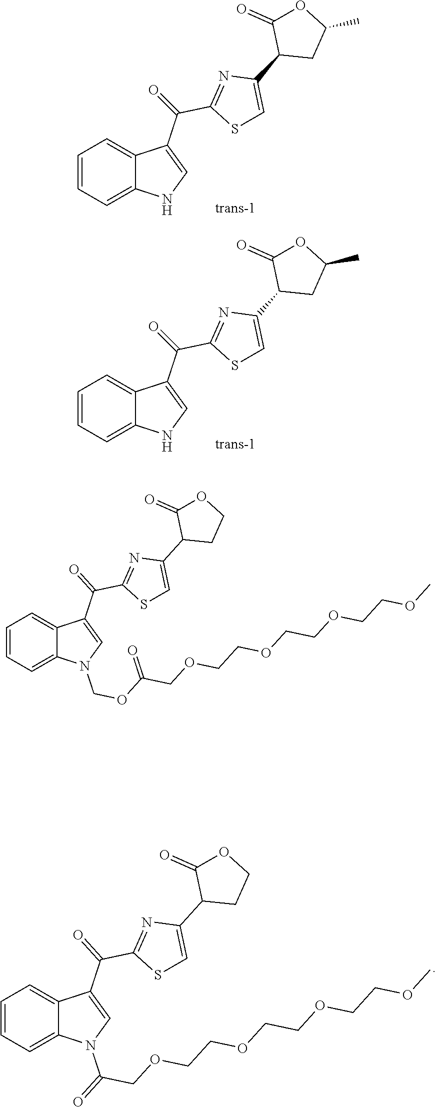 [SMILES: C[C@@H]1C[C@@H](c2csc(C(=O)c3c[nH]c4ccccc34)n2)C(=O)O1 ; C[C@H]1C[C@H](c2csc(C(=O)c3c[nH]c4ccccc34)n2)C(=O)O1 ; COCCOCCOCCOCC(=O)OCn1cc(C(=O)c2nc(C3CCOC3=O)cs2)c2ccccc21 ; COCCOCCOCCOCC(=O)n1cc(C(=O)c2nc(C3CCOC3=O)cs2)c2ccccc21]

Some embodiments of the present disclosure provide a salt, a prodrug, a hydrate, a solvate, or a deuterated derivative further substituted with deuterium of the aryl hydrocarbon receptor modulator of the general formula (I).

Some embodiments of the present disclosure provide a use of the aryl hydrocarbon receptor modulator of the general formula (I) in the preparation of a medicament for treating a central nervous system disease, cancer, or obesity, or in the preparation of a medicament for immunomodulation, hematopoiesis, cell cycle regulation, or intestinal barrier regulation, wherein the central nervous system disease is selected from Alzheimer's disease, Parkinson's disease, and multiple sclerosis; the immunomodulation is selected from immunomodulation for psoriasis, atopic dermatitis, lupus erythematosus, or vitiligo; and the intestinal barrier regulation is for inflammatory bowel disease.

Some embodiments of the present disclosure provide a use of the aryl hydrocarbon receptor modulator of the general formula (I) in the treatment of a central nervous system disease, cancer, or obesity, or in immunomodulation, hematopoiesis, cell cycle regulation, or intestinal barrier regulation, wherein the central nervous system disease is selected from Alzheimer's disease, Parkinson's disease, and multiple sclerosis; the immunomodulation is selected from immunomodulation for psoriasis, atopic dermatitis, lupus erythematosus, or vitiligo; and the intestinal barrier regulation is for inflammatory bowel disease.

Some embodiments of the present disclosure provide a method for preparing a drug for treating a central nervous system disease, cancer, or obesity, or for regulating immunomodulation, hematopoiesis, cell cycle, or intestinal barrier. The method includes mixing the aryl hydrocarbon receptor modulator, or the enantiomer, the prodrug, or the pharmaceutically acceptable salt with at least one pharmaceutically acceptable excipient, wherein the central nervous system disease is selected from Alzheimer's disease, Parkinson's disease, or multiple sclerosis; the immunomodulation is selected from immunomodulation for psoriasis, atopic dermatitis, lupus erythematosus, or vitiligo; and the intestinal barrier is for inflammatory bowel disease.

Some embodiments of the present disclosure provide a method for treating a central nervous system disease, cancer, or obesity, or for regulating immunomodulation, hematopoiesis, cell cycle, or intestinal barrier. The method includes administering the aryl hydrocarbon receptor modulator, or the enantiomer, the prodrug, or the pharmaceutically acceptable salt to a subject, wherein the central nervous system disease is selected from Alzheimer's disease, Parkinson's disease, or multiple sclerosis; the immunomodulation is selected from immunomodulation for psoriasis, atopic dermatitis, lupus erythematosus, or vitiligo; and the intestinal barrier is for inflammatory bowel disease.

A synthesis process of the aryl hydrocarbon receptor modulator of the general formula (I) of the present disclosure is as follows:

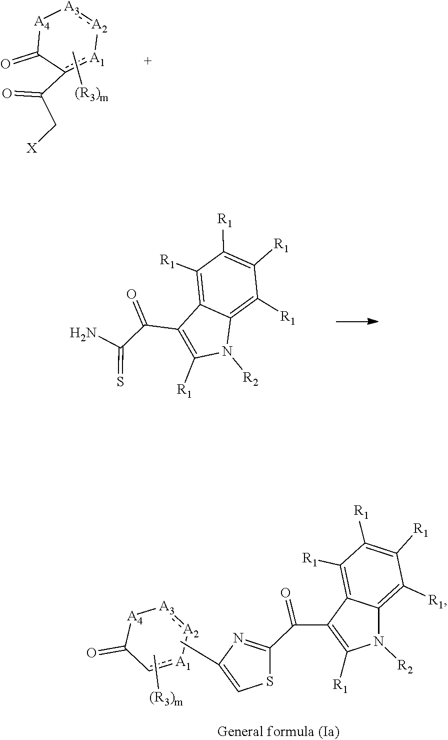

wherein X is halogen.

### DETAILED DESCRIPTION

In the embodiments of the present disclosure, halogen refers to a halogen atom, including fluorine, chlorine, bromine, and iodine. Alkyl refers to a saturated straight or branched chain hydrocarbon group composed of C and H. Alkenyl refers to a straight or branched chain hydrocarbon group containing at least one carbon-carbon double bond. Alkynyl refers to a straight or branched chain hydrocarbon group containing at least one carbon-carbon triple bond. Cycloalkyl refers to a saturated cyclic hydrocarbon group in which all ring atoms are carbon and all carbon-carbon bonds are all single bonds.

Some embodiments of the present disclosure provide an aryl hydrocarbon receptor modulator or an enantiomer, a prodrug, or a pharmaceutically acceptable salt thereof. The aryl hydrocarbon receptor modulator includes the following general formula (I):

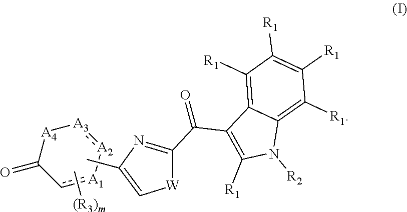

In the general formula (I), [symbol] is independently a single bond or a double bond. In the structures

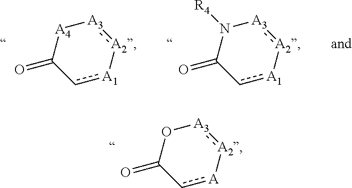

[symbol]

between A₂ and A₃ indicates that the bond between A₂ and A₃ may be either a single bond or a double bond. [symbol] between C and A₁ indicates that the bond between C and A₁ may be a single bond or a double bond. In some embodiments, whether the bond between A₂ and A₃ is a single bond or a double bond is independent of whether the bond between C and A₁ is a single bond or a double bond, there is no necessary correlation between the two, and it only needs to comply with the basic principle of chemical bond saturation. For example, when [symbol] between A₂ and A₃ is a single bond, [symbol] between C and A₁ may be a single bond or a double bond.

In some embodiments, in the general formula (I), a substituent

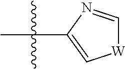

may be attached to any substitutable position on the structures

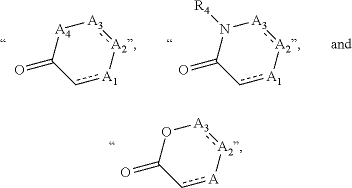

i.e., the substituent

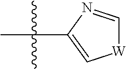

may be attached to C, or may be attached to A₁, A₂, or A₃.

In some embodiments, in the general formula (I), —(R₃)ₘ may be attached to any substitutable position on the structures

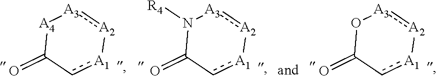

i.e., —(R₃)ₘ may be attached to C, or may be attached to A₁, A₂, or A₃. For example, there may be one or more (e.g., at least two) R₃. In some embodiments, at least two R₃ groups may be attached to the same atom or to different atoms.

In some embodiments, m is an integer from 1 to 7. In some embodiments, m is an integer from 1 to 5. In some embodiments, m is an integer from 1 to 3. In some embodiments, m is an integer from 1 to 2. For example, m is 1, 2, 3, 4, 5, 6, or 7.

In some embodiments, R₃ is independently H, D, halogen, CN, OR, SR, N(R)R, —C(O)R, —C(O)OR, RC(O)O—, S(O)ₙR, unsubstituted C1-C8 alkyl or substituted C1-C8 alkyl with 1-6 Rₐ, unsubstituted C2-C8 alkenyl or substituted C2-C8 alkenyl with 1-6 Rₐ, unsubstituted C2-C8 alkynyl or substituted C2-C8 alkynyl with 1-6 Rₐ, or unsubstituted C3-C10 cycloalkyl or substituted C3-C10 cycloalkyl with 1-6 Rₐ. In some embodiments, R₃ is independently H, D, halogen, CN, OR, SR, N(R)R, —C(O)R, —C(O)OR, RC(O)O—, S(O)ₙR, unsubstituted C1-C6 alkyl or substituted C1-C6 alkyl with 1-3 Rₐ, unsubstituted C2-C6 alkenyl OR substituted C2-C6 alkenyl with 1-3 Rₐ, unsubstituted C2-C6 alkynyl or substituted C2-C6 alkynyl with 1-3 Rₐ, or unsubstituted C3-C8 cycloalkyl or substituted C3-C8 cycloalkyl with 1-3 Rₐ. In some embodiments, R₃ is independently H, D, halogen, CN, OR, SR, N(R)R, —C(O)R, —C(O)OR, RC(O)O—, S(O)ₙR, unsubstituted C1-C4 alkyl or substituted C1-C4 alkyl with 1-2 Rₐ, unsubstituted C2-C4 alkenyl or substituted C2-C4 alkenyl with 1-2 Rₐ, unsubstituted C2-C4 alkynyl or substituted C2-C4 alkynyl with 1-2 Rₐ, or unsubstituted C3-C7 cycloalkyl or substituted C3-C7 cycloalkyl with 1-2 Rₐ. In some embodiments, R₃ is independently H, D, halogen, CN, OR, SR, N(R)R, —C(O)R, —C(O)OR, RC(O)O—, S(O)ₙR, unsubstituted C1-C3 alkyl or substituted C1-C3 alkyl with 1 Rₐ, unsubstituted C2-C3 alkenyl or substituted C2-C3 alkenyl with 1 Rₐ, unsubstituted C2-C3 alkynyl or substituted C2-C3 alkynyl with 1 Rₐ, or unsubstituted C5-C6 cycloalkyl or substituted C5-C6 cycloalkyl with 1 Rₐ.

In some embodiments, Rₐ is independently D, halogen, OR, SR, N(R)R, —C(O)R, —C(O)OR, RC(O)O—, or S(O)ₙR.

In some embodiments, R is independently H, C1-C8 alkyl, C2-C8 alkenyl, C2-C8 alkynyl, or C3-C10 cycloalkyl. In some embodiments, R is independently H, C1-C6 alkyl, C2-C6 alkenyl, C2-C6 alkynyl, or C3-C8 cycloalkyl. In some embodiments, R is independently H, C1-C4 alkyl, C2-C4 alkenyl, C2-C4 alkynyl, or C3-C7 cycloalkyl. In some embodiments, R is independently H, C1-C3 alkyl, C2-C3 alkenyl, C2-C3 alkynyl, or C5-C6 cycloalkyl.

n may be 1 or 2.

A₁, A₂, and A₃ may be each independently a bond or C. The bond may be a single bond or a double bond. For example, when A₁ is the bond, C on one side of A₁ is connected to A₂ on the other side of A₁ through a single bond or a double bond. For example, when A₂ is the bond, A₁ on one side of A₂ is connected to A₃ on the other side of A₂ through a single bond or a double bond. For example, when A₃ is the bond, A₂ on one side of A₃ is connected to A₄ on the other side of A₃ through a single bond or a double bond. For example, when both A₁ and A₂ are the bonds, C on the other side of A₁ is connected to A₃ on the other side of A₂ through a single bond or a double bond. For example, when A₁, A₂, and A₃ are the bonds, C on the other side of A₁ is connected to A₄ on the other side of A₃ through a single bond or a double bond.

A₄ may be independently O or NR₄.

In some embodiments, R₄ is H, unsubstituted C1-C8 alkyl or substituted C1-C8 alkyl with 1-6 R_{b}, unsubstituted C2-C8 alkenyl or substituted C2-C8 alkenyl with 1-6 R_{b}, unsubstituted C2-C8 alkynyl or substituted C2-C8 alkynyl with 1-6 R_{b}, or unsubstituted C3-C10 cycloalkyl or substituted C3-C10 cycloalkyl with 1-6 R_{b}. In some embodiments, R₄ is H, unsubstituted C1-C6 alkyl or substituted C1-C6 alkyl with 1-3 R_{b}, unsubstituted C2-C6 alkenyl or substituted C2-C6 alkenyl with 1-3 R_{b}, unsubstituted C2-C6 alkynyl or substituted C2-C6 alkynyl with 1-3 R_{b}, or unsubstituted C3-C8 cycloalkyl or substituted C3-C8 cycloalkyl with 1-3 R_{b}. In some embodiments, R₄ is H, unsubstituted C1-C4 alkyl or substituted C1-C4 alkyl with 1-2 R_{b}, unsubstituted C2-C4 alkenyl or substituted C2-C4 alkenyl with 1-2 R_{b}, unsubstituted C2-C4 alkynyl or substituted C2-C4 alkynyl with 1-2 R_{b}, or unsubstituted C3-C7 cycloalkyl or substituted C3-C7 cycloalkyl with 1-2 R_{b}. In some embodiments, R₄ is H, unsubstituted C1-C3 alkyl or substituted C1-C3 alkyl with 1 R_{b}, unsubstituted C2-C3 alkenyl or substituted C2-C3 alkenyl with 1 R_{b}, unsubstituted C2-C3 alkynyl or substituted C2-C3 alkynyl with 1 R_{b}, or unsubstituted C5-C6 cycloalkyl or substituted C5-C6 cycloalkyl with 1 R_{b}.

R_{b} may be independently D, halogen, OR, SR, N(R)R, —C(O)R, —C(O)OR, RC(O)O—, or S(O)ₙR. More descriptions regarding R and n may be found in the foregoing descriptions of the present disclosure.

W may be C═C or S.

In some embodiments, and R₁ is independently H, D, halogen, CN, OR, SR, N(R)R, —C(O)R, —C(O)OR, RC(O)O—, S(O)ₙR, unsubstituted C1-C8 alkyl or substituted C1-C8 alkyl with 1-6 R_{b}, unsubstituted C2-C8 alkenyl or substituted C2-C8 alkenyl with 1-6 R_{b}, unsubstituted C2-C8 alkynyl or substituted C2-C8 alkynyl with 1-6 R_{b}, unsubstituted C3-C10 cycloalkyl or substituted C3-C10 cycloalkyl with 1-6 R_{b}, or two adjacent or meta R₁ groups form a 3-10 membered carbocycle or a 3-10 membered heterocycle containing 1 to 3 heteroatoms selected from N, O, or S. In some embodiments, R₁ is independently H, D, halogen, CN, OR, SR, N(R)R, —C(O)R, —C(O)OR, RC(O)O—, S(O)ₙR, unsubstituted C1-C6 alkyl or substituted C1-C6 alkyl with 1-3 R_{b}, unsubstituted C2-C6 alkenyl or substituted C2-C6 alkenyl with 1-3 R_{b}, unsubstituted C2-C6 alkynyl or substituted C2-C6 alkynyl with 1-3 R_{b}, unsubstituted C3-C8 cycloalkyl or substituted C3-C8 cycloalkyl with 1-3 R_{b}, or two adjacent or meta R₁ groups form a 3-8 membered carbocycle or a 3-8 membered heterocycle containing 1-3 heteroatoms selected from N, O, or S. In some embodiments, R₁ is independently H, D, halogen, CN, OR, SR, N(R)R, —C(O)R, —C(O)OR, RC(O)O—, S(O)ₙR, unsubstituted C1-C4 alkyl or substituted C1-C4 alkyl with 1-2 R_{b}, unsubstituted C2-C4 alkenyl or substituted C2-C4 alkenyl with 1-2 R_{b}, unsubstituted C2-C4 alkynyl or substituted C2-C4 alkynyl with 1-2 R_{b}, unsubstituted C3-C7 cycloalkyl or substituted C3-C7 cycloalkyl with 1-2 R_{b}, or two adjacent or meta R₁ groups form a 3-7 membered carbocycle or a 3-7 membered heterocycle containing 1-2 heteroatoms selected from N, O, or S. In some embodiments, R₁ is independently H, D, halogen, CN, OR, SR, N(R)R, —C(O)R, —C(O)OR, RC(O)O—, S(O)ₙR, unsubstituted C1-C3 alkyl or substituted C1-C3 alkyl with 1 R_{b}, unsubstituted C2-C3 alkenyl or substituted C2-C3 alkenyl with 1 R_{b}, unsubstituted C2-C3 alkynyl or substituted C2-C3 alkynyl with 1 R_{b}, or unsubstituted C5-C6 cycloalkyl or substituted C5-C6 cycloalkyl with 1 R_{b}, or two adjacent or meta R₁ groups form a 5-6 membered carbocycle or a 5-6 membered heterocycle containing 1-2 heteroatoms selected from N, O, or S. More descriptions regarding R, n and R_{b} may be found in the foregoing descriptions of the present disclosure.

The carbocycle refers to a cyclic structure whose skeleton is entirely composed of carbon atoms. The carbon-carbon bond in the carbocycle may be a single bond, a double bond, or a triple bond. The 3-10 membered carbocycle refers to a ring formed by 3-10 carbon atoms.

The heterocycle refers to a cyclic structure whose skeleton contains at least one non-carbon atom. The non-carbon atom (also referred to as a heteroatom) may be selected from at least one of N, O, or S. The carbon-carbon bond and the bond between C and the heteroatom in the heterocycle may be single bonds, double bonds, or triple bonds.

The 3-10 membered heterocycle containing 1-3 heteroatoms selected from N, O, and S refers to a ring having 3-10 skeleton atoms, where the skeleton atoms forming the ring include C and heteroatoms, the heteroatoms are selected from at least one of N, O, or S, and a count of the heteroatoms is 1-3. For example, the heteroatoms may be N, O, S, two N, one N and one O, one O and one S, two N and one S, as well as one N, one O, and one S, etc.

R₂ may be H, —(CH₂O)ₐC(O)(CH₂)_{b}(O)_{c}(CH(R_{c}))_{d}(CH₂CH₂O)ₑR_{c}, —(CH₂O)ₐP(O)(Rₚ)₂, or —(CH₂O)ₐ(CH(R_{c}))_{d}(CH₂CH₂O)ₑR_{c}.

Rₚ may be independently selected form (CH₂)_{b}(O)_{c}(CH(R_{c}))_{d}(CH₂CH₂O)ₑR_{c}.

In some embodiments, R_{c} is independently H, D, C1-C8 alkyl, NH₂—, (C1-C8 alkyl)NH—, (C1-C8 alkyl)₂N—, NH₂(C1-C8 alkyl)-, OH, HO—(C1-C8 alkyl)-, or (C1-C8 alkyl)O—. In some embodiments, R_{c} is independently H, D, C1-C6 alkyl, NH₂—, (C1-C6 alkyl)NH—, (C1-C6 alkyl)₂N—, NH₂(C1-C6 alkyl)-, OH, HO—(C1-C6 alkyl)-, or (C1-C6 alkyl)O—. In some embodiments, R_{c} is independently H, D, C1-C4 alkyl, NH₂—, (C1-C4 alkyl)NH—, (C1-C4 alkyl)₂N—, NH₂(C1-C4)alkyl-, OH, HO—(C1-C4)alkyl-, or (C1-C4 alkyl)O—. In some embodiments, R_{c} is independently H, D, C1-C3 alkyl, NH₂—, (C1-C3 alkyl)NH—, (C1-C3 alkyl)₂N—, NH₂(C1-C3)alkyl-, OH, HO—(C1-C3)alkyl-, or (C1-C3 alkyl)O—.

a, b, c, and d may be each independently 0 or 1. In some embodiments of the present disclosure, a, b, and c denote the number of —CH₂O—, —CH₂—, and —O— in R₂ or Rₚ, respectively, and are unrelated to Rₐ, R_{b}, and R_{c}.

In some embodiments, e is an integer from 0 to 3. In some embodiments, e is an integer from 0 to 2. In some embodiments, e is an integer of 0 or 1. For example, e is 0, 1, 2, 3, etc.

In some embodiments, when W is S, the general formula (I) is a general formula (Ia):

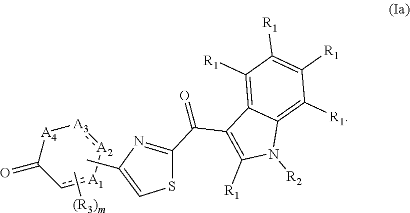

In some embodiments, when A₄ is O, the general formula (Ia) is a general formula (Ia1):

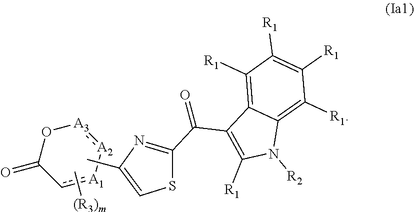

In some embodiments, when A₄ is NR₄, the general formula (Ia) is a general formula (Ia2):

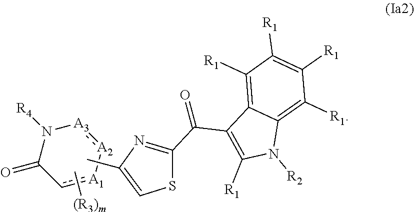

In some embodiments, when W is C═C, the general formula (I) is a general formula (Ib):

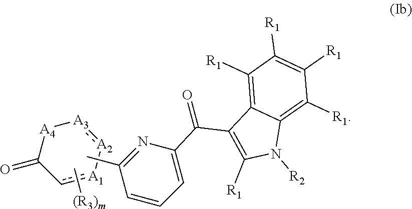

In some embodiments, when A₄ is O, the general formula (Ib) is a general formula (Ib1):

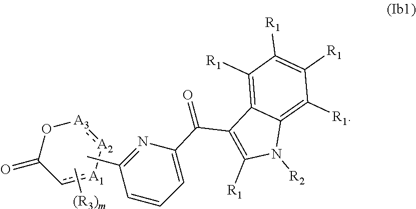

In some embodiments, when A₄ is NR₄, the general formula (Ib) is a general formula (Ib2):

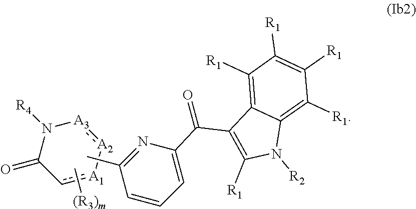

In some embodiments, the aryl hydrocarbon receptor modulator is one of the following compounds:

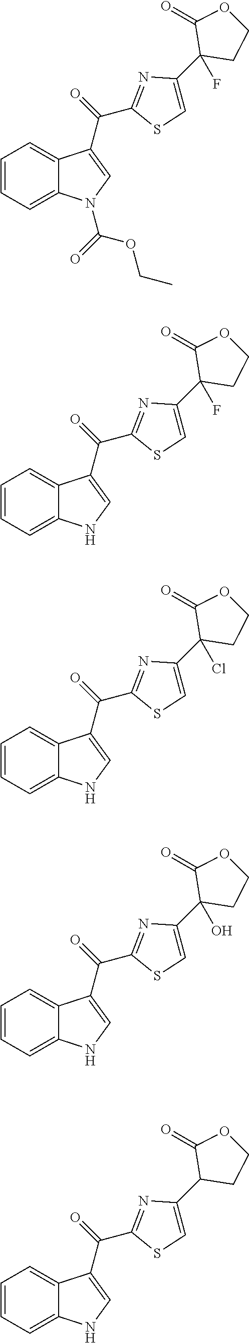 [SMILES: CCOC(=O)n1cc(C(=O)c2nc(C3(F)CCOC3=O)cs2)c2ccccc21 ; O=C(c1nc(C2(F)CCOC2=O)cs1)c1c[nH]c2ccccc12 ; O=C(c1nc(C2(Cl)CCOC2=O)cs1)c1c[nH]c2ccccc12 ; O=C(c1nc(C2(O)CCOC2=O)cs1)c1c[nH]c2ccccc12 ; O=C(c1nc(C2CCOC2=O)cs1)c1c[nH]c2ccccc12]

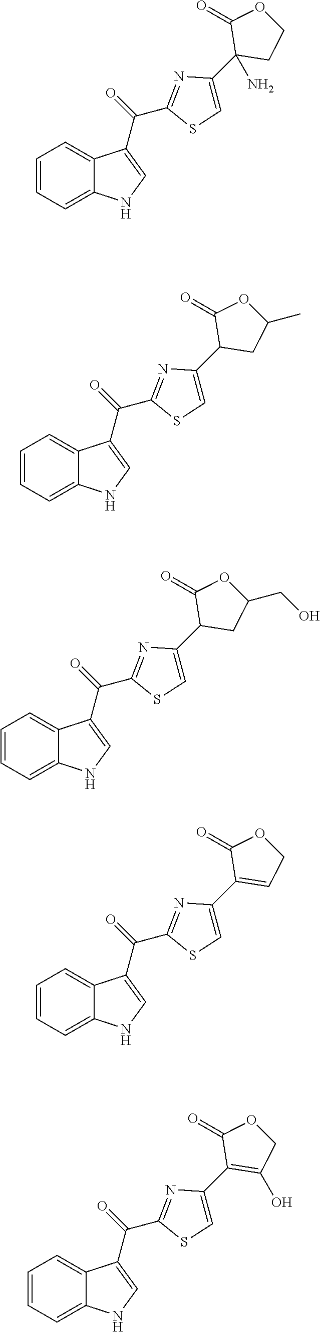 [SMILES: NC1(c2csc(C(=O)c3c[nH]c4ccccc34)n2)CCOC1=O ; CC1CC(c2csc(C(=O)c3c[nH]c4ccccc34)n2)C(=O)O1 ; O=C(c1nc(C2CC(CO)OC2=O)cs1)c1c[nH]c2ccccc12 ; O=C1OCC=C1c1csc(C(=O)c2c[nH]c3ccccc23)n1 ; O=C1OCC(O)=C1c1csc(C(=O)c2c[nH]c3ccccc23)n1]

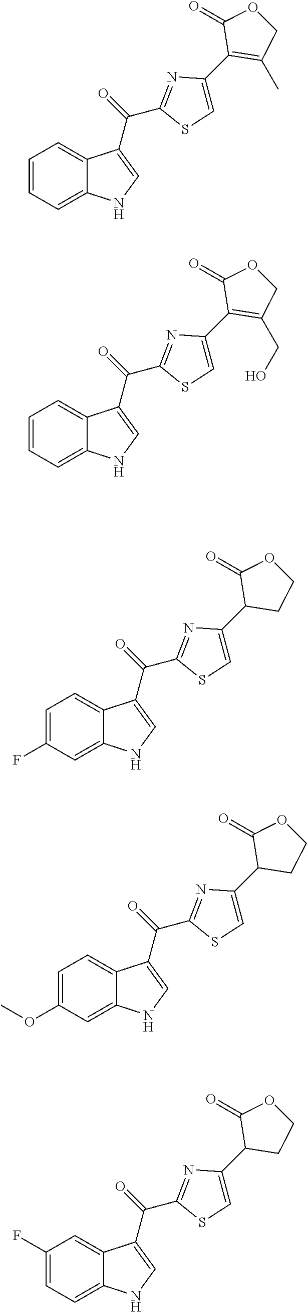

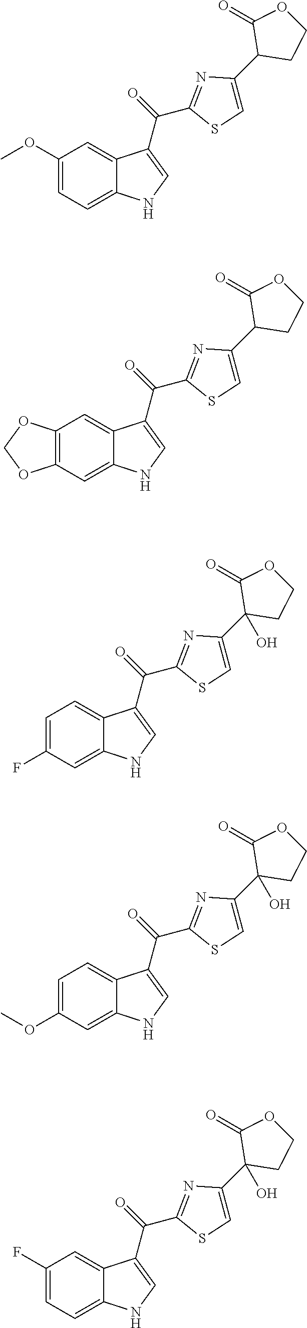 [SMILES: COc1ccc2[nH]cc(C(=O)c3nc(C4CCOC4=O)cs3)c2c1 ; O=C(c1nc(C2CCOC2=O)cs1)c1c[nH]c2cc3c(cc12)OCO3 ; O=C(c1nc(C2(O)CCOC2=O)cs1)c1c[nH]c2cc(F)ccc12 ; COc1ccc2c(C(=O)c3nc(C4(O)CCOC4=O)cs3)c[nH]c2c1 ; O=C(c1nc(C2(O)CCOC2=O)cs1)c1c[nH]c2ccc(F)cc12]

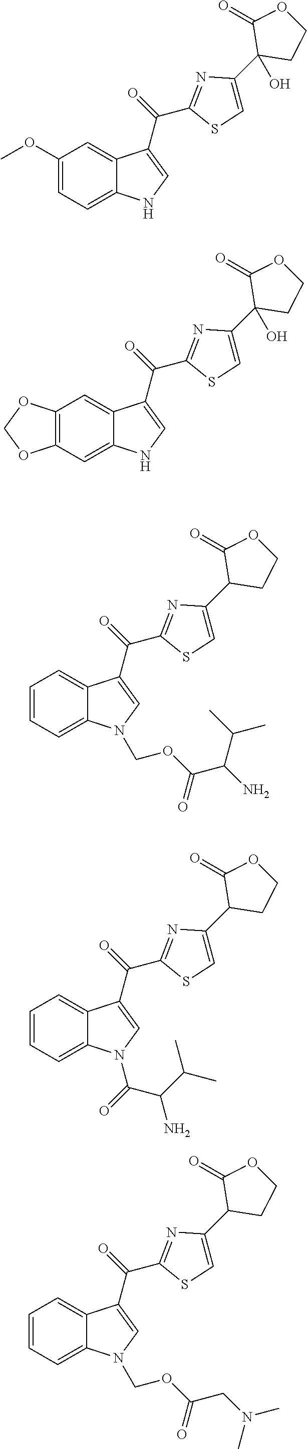

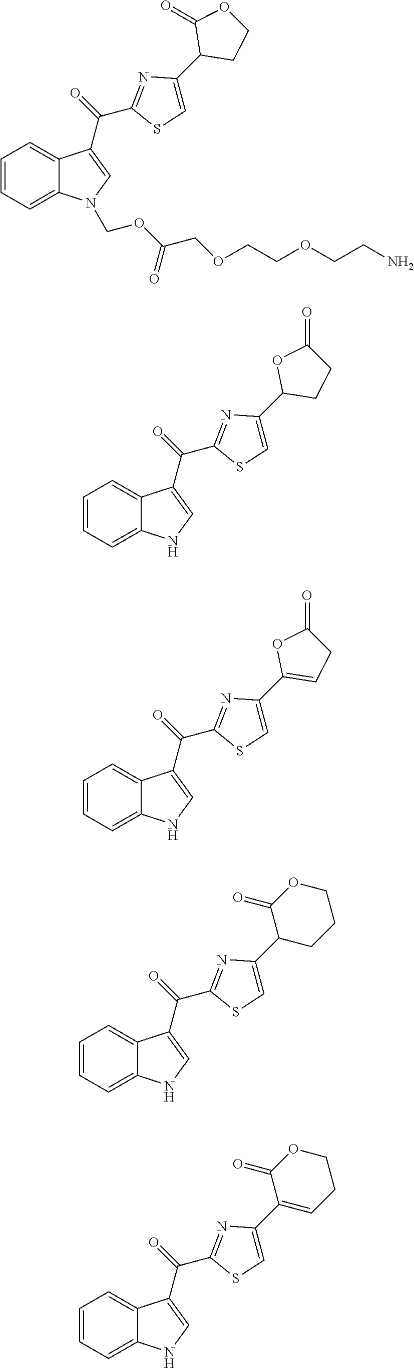 [SMILES: NCCOCCOCC(=O)OCn1cc(C(=O)c2nc(C3CCOC3=O)cs2)c2ccccc21 ; O=C1CCC(c2csc(C(=O)c3c[nH]c4ccccc34)n2)O1 ; O=C1CC=C(c2csc(C(=O)c3c[nH]c4ccccc34)n2)O1 ; O=C(c1nc(C2CCCOC2=O)cs1)c1c[nH]c2ccccc12 ; O=C1OCCC=C1c1csc(C(=O)c2c[nH]c3ccccc23)n1]

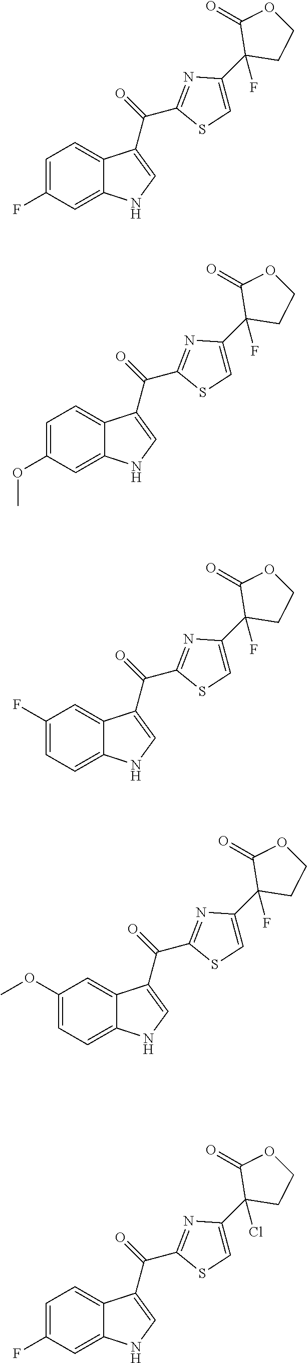 [SMILES: O=C(c1nc(C2(F)CCOC2=O)cs1)c1c[nH]c2cc(F)ccc12 ; COc1ccc2c(C(=O)c3nc(C4(F)CCOC4=O)cs3)c[nH]c2c1 ; O=C(c1nc(C2(F)CCOC2=O)cs1)c1c[nH]c2ccc(F)cc12 ; COc1ccc2[nH]cc(C(=O)c3nc(C4(F)CCOC4=O)cs3)c2c1 ; O=C(c1nc(C2(Cl)CCOC2=O)cs1)c1c[nH]c2cc(F)ccc12]

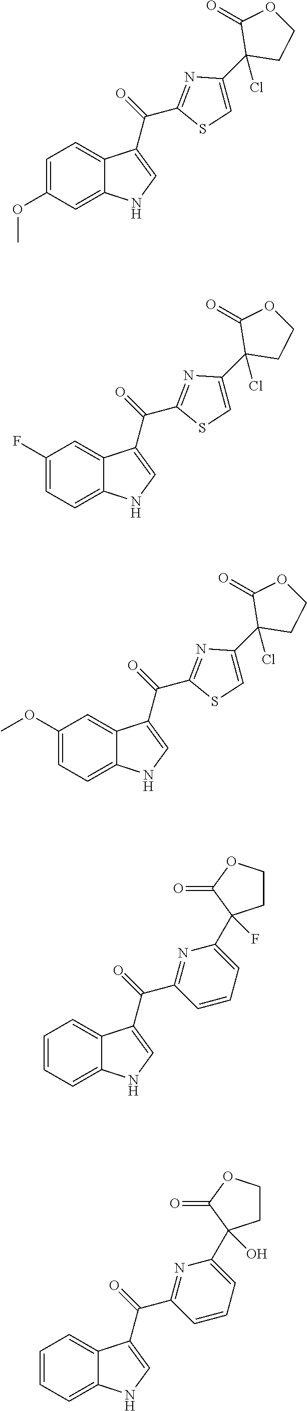 [SMILES: COc1ccc2c(C(=O)c3nc(C4(Cl)CCOC4=O)cs3)c[nH]c2c1 ; O=C(c1nc(C2(Cl)CCOC2=O)cs1)c1c[nH]c2ccc(F)cc12 ; COc1ccc2[nH]cc(C(=O)c3nc(C4(Cl)CCOC4=O)cs3)c2c1 ; O=C(c1cccc(C2(F)CCOC2=O)n1)c1c[nH]c2ccccc12 ; O=C(c1cccc(C2(O)CCOC2=O)n1)c1c[nH]c2ccccc12]

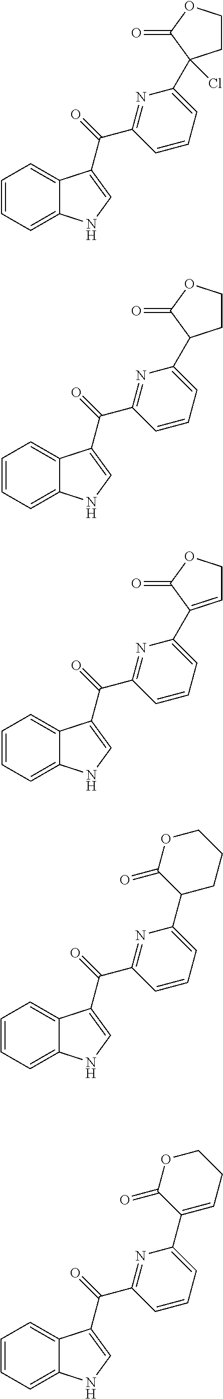 [SMILES: O=C(c1cccc(C2(Cl)CCOC2=O)n1)c1c[nH]c2ccccc12 ; O=C(c1cccc(C2CCOC2=O)n1)c1c[nH]c2ccccc12 ; O=C1OCC=C1c1cccc(C(=O)c2c[nH]c3ccccc23)n1 ; O=C(c1cccc(C2CCCOC2=O)n1)c1c[nH]c2ccccc12 ; O=C1OCCC=C1c1cccc(C(=O)c2c[nH]c3ccccc23)n1]

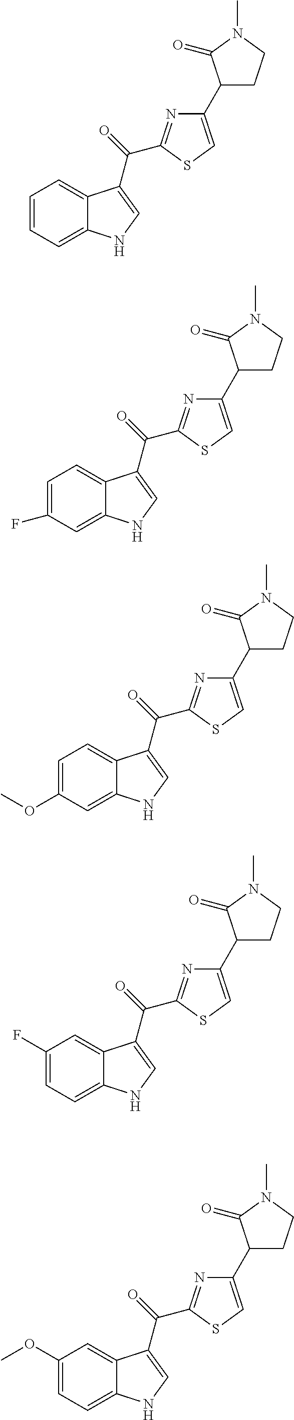 [SMILES: CN1CCC(c2csc(C(=O)c3c[nH]c4ccccc34)n2)C1=O ; CN1CCC(c2csc(C(=O)c3c[nH]c4cc(F)ccc34)n2)C1=O ; COc1ccc2c(C(=O)c3nc(C4CCN(C)C4=O)cs3)c[nH]c2c1 ; CN1CCC(c2csc(C(=O)c3c[nH]c4ccc(F)cc34)n2)C1=O ; COc1ccc2[nH]cc(C(=O)c3nc(C4CCN(C)C4=O)cs3)c2c1]

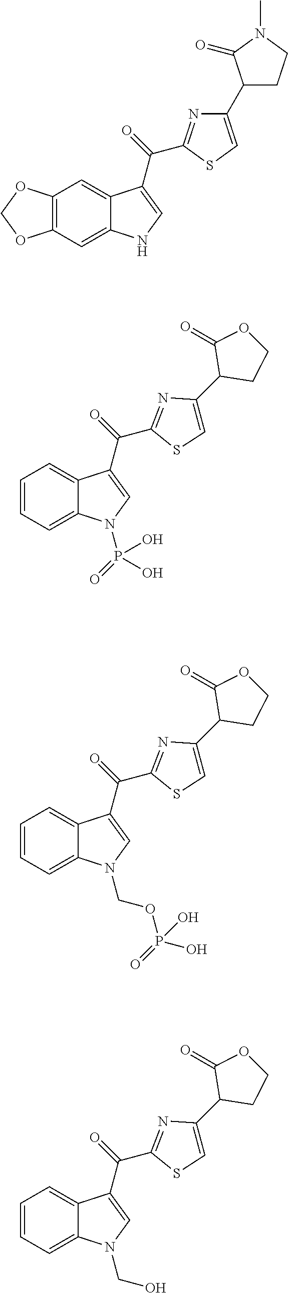 [SMILES: CN1CCC(c2csc(C(=O)c3c[nH]c4cc5c(cc34)OCO5)n2)C1=O ; O=C(c1nc(C2CCOC2=O)cs1)c1cn(P(=O)(O)O)c2ccccc12 ; O=C(c1nc(C2CCOC2=O)cs1)c1cn(COP(=O)(O)O)c2ccccc12 ; O=C(c1nc(C2CCOC2=O)cs1)c1cn(CO)c2ccccc12]

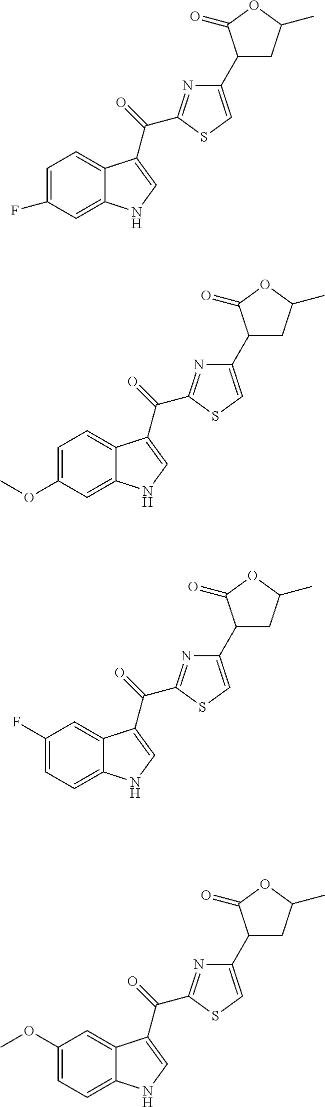

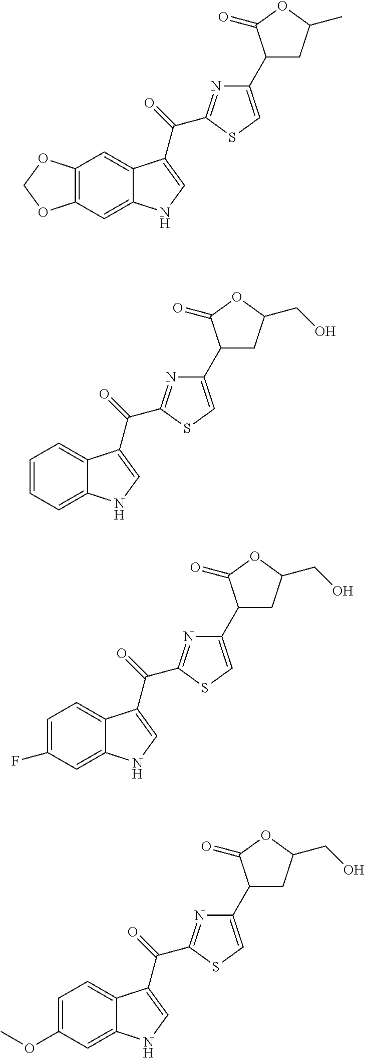

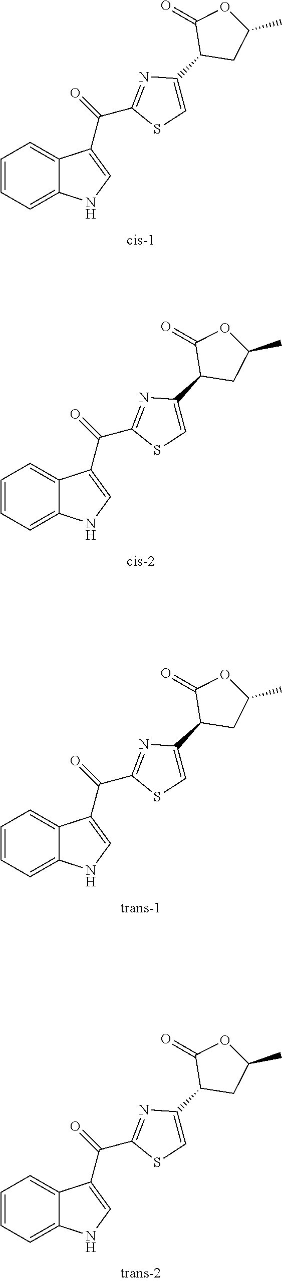 [SMILES: C[C@@H]1C[C@H](c2csc(C(=O)c3c[nH]c4ccccc34)n2)C(=O)O1 ; C[C@H]1C[C@@H](c2csc(C(=O)c3c[nH]c4ccccc34)n2)C(=O)O1 ; C[C@@H]1C[C@@H](c2csc(C(=O)c3c[nH]c4ccccc34)n2)C(=O)O1 ; C[C@H]1C[C@H](c2csc(C(=O)c3c[nH]c4ccccc34)n2)C(=O)O1]

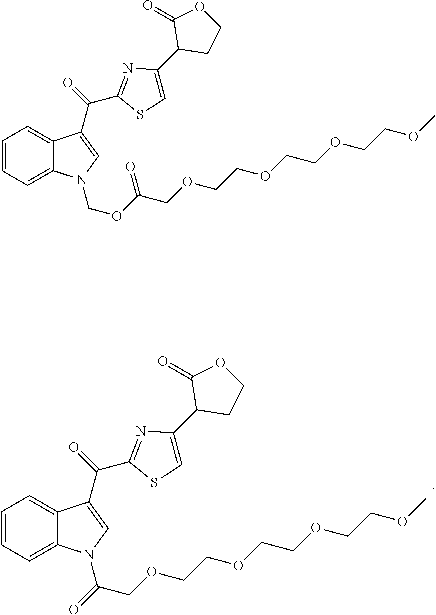 [SMILES: COCCOCCOCCOCC(=O)OCn1cc(C(=O)c2nc(C3CCOC3=O)cs2)c2ccccc21 ; COCCOCCOCCOCC(=O)n1cc(C(=O)c2nc(C3CCOC3=O)cs2)c2ccccc21]

Some embodiments of the present disclosure provide a salt, a prodrug, a hydrate, a solvate, or a deuterated derivative further substituted with deuterium of the aryl hydrocarbon receptor modulator.

The salt of the aryl hydrocarbon receptor modulator refers to an ionic compound formed by reacting the compound of the general formula (I) with an acid or a base.

The prodrug of the aryl hydrocarbon receptor modulator refers to a derivative obtained by chemically modifying the compound of the general formula (I). The derivative is inactive or has low activity in vitro, and is converted in vivo (through an enzymatic, pH, or a metabolic process) to release the active parent drug (i.e., the compound of the general formula (I)). The chemically modifying may include, but is not limited to, attaching a cleavable group (e.g., an ester, an amide, a phosphate ester, or a carbamate) to groups like —OH, —NH₂, etc.

The hydrate of the aryl hydrocarbon receptor modulator refers to a solid form formed during a crystallization process of the compound of the general formula (I), where water molecules are incorporated into the crystal lattice as part of its crystal structure.

The solvate of the aryl hydrocarbon receptor modulator refers to a solid form formed during a crystallization process of the compound of the general formula (I), where organic solvent molecules are incorporated into the crystal lattice. The organic solvent molecules may include, but are not limited to, ethanol, methanol, acetone, or ethyl acetate.

The deuterated derivative further substituted with deuterium of the aryl hydrocarbon receptor modulator refers to a compound obtained by replacing one or more H in the compound of the general formula (I) with deuterium atom (D).

Some embodiments of the present disclosure provide a method for preparing a drug for treating a central nervous system disease, cancer, or obesity, or for regulating immunomodulation, hematopoiesis, cell cycle, or intestinal barrier. The method includes mixing the aryl hydrocarbon receptor modulator, or the enantiomer, the prodrug, or the pharmaceutically acceptable salt with at least one pharmaceutically acceptable excipient, wherein the central nervous system disease is selected from Alzheimer's disease, Parkinson's disease, or multiple sclerosis; the immunomodulation is selected from immunomodulation for psoriasis, atopic dermatitis, lupus erythematosus, or vitiligo; and the intestinal barrier is for inflammatory bowel disease. In some embodiments, the at least one pharmaceutically acceptable excipient may include, but are not limited to, a filler (e.g., lactose or starch), a binder (e.g., povidone or hypromellose), a disintegrant (e.g., croscarmellose sodium), a lubricant (e.g., magnesium stearate or talc), and an antioxidant (e.g., sodium bisulfite or vitamin E), etc. In some embodiments, the at least one pharmaceutically acceptable excipient may account for 30 wt % to 90 wt % of the drug.

Some embodiments of the present disclosure provide a method for treating a central nervous system disease, cancer, or obesity, or for regulating immunomodulation, hematopoiesis, cell cycle, or intestinal barrier. The method includes administering the aryl hydrocarbon receptor modulator, or the enantiomer, the prodrug, or the pharmaceutically acceptable salt to a subject, wherein the central nervous system disease is selected from Alzheimer's disease, Parkinson's disease, or multiple sclerosis; the immunomodulation is selected from immunomodulation for psoriasis, atopic dermatitis, lupus erythematosus, or vitiligo; and the intestinal barrier is for inflammatory bowel disease.

### Synthesis of Intermediate Int-1

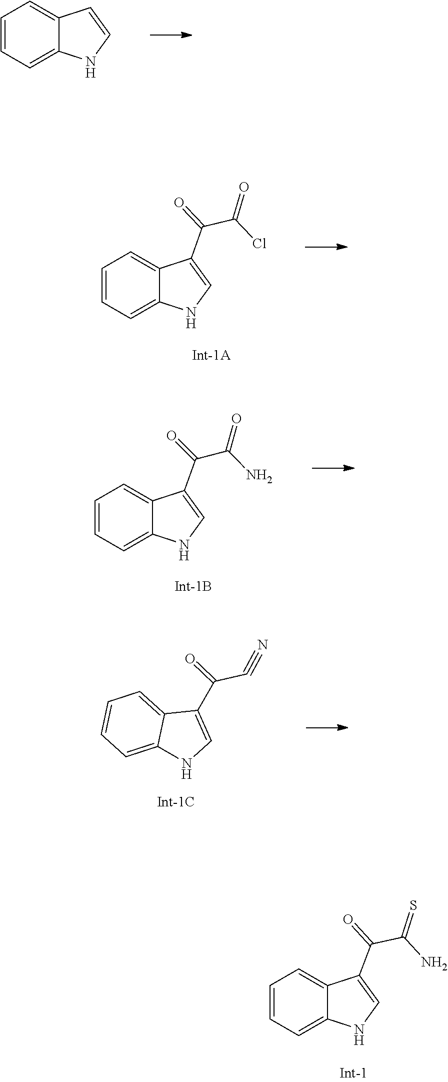 [SMILES: c1ccc2[nH]ccc2c1 ; O=C(Cl)C(=O)c1c[nH]c2ccccc12 ; NC(=O)C(=O)c1c[nH]c2ccccc12 ; N#CC(=O)c1c[nH]c2ccccc12 ; NC(=S)C(=O)c1c[nH]c2ccccc12]

### Step 1: 2-(1H-indol-3-yl)-2-oxalyl chloride (also Referred to as Int-1A)

At room temperature, indole (131 g, 1118 mmol) was dissolved in methyl tert-butyl ether (MTBE) (983 mL). The mixture was cooled to −10° C. Oxalyl chloride (149 g, 1174 mmol) was added dropwise slowly, and a temperature of −10° C. to 5° C. was maintained during the addition. After the addition was complete, the mixture was allowed to warm to room temperature naturally and reacted for 1 hour. Petroleum ether (983 mL) was added. The mixture was stirred at room temperature for 30 minutes and then filtered. The filter cake was washed with petroleum ether (200 mL). The solid filter cake was dried under reduced pressure to obtain a yellow solid Int-1A (228 g).

### Step 2: 2-(1H-indol-3-yl)-2-oxalamide (also Referred to as Int-1B)

Aqueous ammonia (~25 wt %, 749 mL) and ethanol (1140 mL) were mixed and cooled to −5° C. to 14° C. The Int-1A (228 g, 1101 mmol) was then added slowly in portions. After the addition was complete, the reaction mixture was maintained at −5° C. to 14° C. for 2 hours. The reaction mixture was poured into water (1140 mL). The mixture was stirred at room temperature for 30 minutes and then filtered. The filter cake was washed with water and dried in an oven at 60° C. overnight to obtain a white solid product Int-1B (170 g).

### Step 3: 1H-indole-3-carbonyl cyanide (also Referred to as Int-1C)

The Int-1B (170 g, 904 mmol) was dissolved in ethyl acetate (2000 mL). Pyridine (214 g, 2712 mmol) was added at room temperature. The mixture was cooled to 0° C. to 5° C. Trifluoroacetic anhydride (284 g, 1356 mmol) was added dropwise slowly over 1.5 hours. The reaction mixture was then maintained at 5° C. to 18° C. for an additional 1.5 hours. After completion of the reaction, the mixture was quenched with a saturated aqueous sodium bicarbonate solution (1700 mL) and stirred for 10 minutes. The mixture was extracted with ethyl acetate twice. The combined organic phases were washed twice with 0.5 mol/L dilute hydrochloric acid (650 mL×2), a volume of the dilute hydrochloric acid used each time was 650 mL and then washed with saturated brine (650 mL) once, dried over anhydrous sodium sulfate, and concentrated to dryness to obtain a crude product. The crude product was treated with a mixture of petroleum ether (PE) and ethyl acetate (EA) (a volume ratio of PE to EA was 10/1, and a volume of the mixture of PE and EA was 400 mL) and filtered to obtain a brown solid Int-1C (145 g).

### Step 4: Intermediate Int-1

Triethylamine (88.4 g, 873 mmol) was added to a solution of the Int-1C (135 g, 794 mmol) in pyridine (1350 mL) at 60° C. Ammonium sulfide (108 g, 1588 mmol) was then added. The reaction mixture was maintained at 60° C. for 1.5 hours. The reaction mixture was then cooled to room temperature and slowly poured into 1 mol/L hydrochloric acid (7 L). The mixture was extracted three times with ethyl acetate (2 L×3), and a volume of the ethyl acetate used each time was 2 L. The combined organic phases were washed with saturated brine (2 L), dried over anhydrous sodium sulfate, filtered, and concentrated to dryness to obtain a crude product of the Int-1. The crude Int-1 was stirred and washed twice with a mixture of PE and EA (a volume ratio of PE to EA was 10/1, and a volume of the mixture of PE and EA was 500 mL) and filtered to obtain a yellow solid Int-1 (90 g).

LCMS [M+H]⁺=205.2.

¹H NMR (400 MHz, DMSO-d₆) δ 12.15 (s, 1H), 10.23 (s, 1H), 10.02 (s, 1H), 8.20 (d, J=3.2 Hz, 1H), 8.14 (dd, J=5.8, 3.2 Hz, 1H), 7.58-7.47 (m, 1H), 7.31-7.19 (m, 2H).

TABLE 1
PREPARATION OF INTERMEDIATES INT-2 TO INT-9 BY THE SAME
METHOD AS THAT FOR PREPARING INTERMEDIATE INT-1

| | | | LCMS |
|---|---|---|---|
| Intermediate | Starting Material | Structural Formula | (ESI): [M + H]⁺ |
| Int-2 | 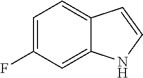 | 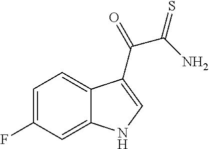 [SMILES: NC(=S)C(=O)c1c[nH]c2cc(F)ccc12] | 331 |
| Int-3 | 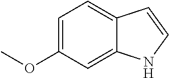 | 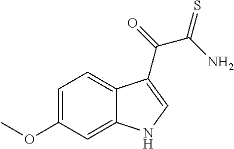 [SMILES: COc1ccc2c(C(=O)C(N)=S)c[nH]c2c1] | 343 |
| Int-4 | 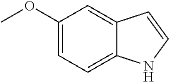 | 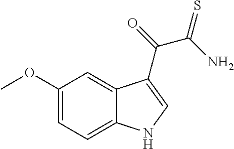 [SMILES: COc1ccc2[nH]cc(C(=O)C(N)=S)c2c1] | 343 |
| Int-5 | 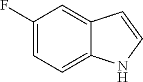 | 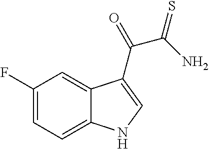 [SMILES: NC(=S)C(=O)c1c[nH]c2ccc(F)cc12] | 331 |
| Int-6 | 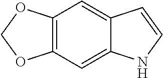 [SMILES: c1cc2cc3c(cc2[nH]1)OCO3] | 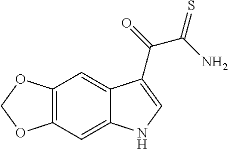 [SMILES: NC(=S)C(=O)c1c[nH]c2cc3c(cc12)OCO3] | 357 |
| Int-7 | 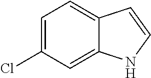 | 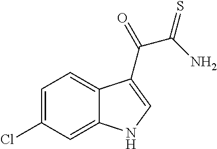 [SMILES: NC(=S)C(=O)c1c[nH]c2cc(Cl)ccc12] | 239 |
| Int-8 | 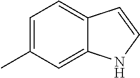 | 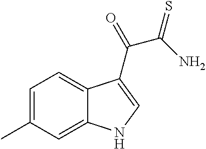 | 219 |
| Int-9 | 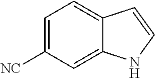 [SMILES: N#Cc1ccc2cc[nH]c2c1] | 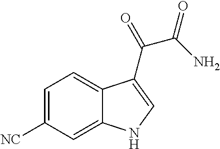 [SMILES: N#Cc1ccc2c(C(=O)C(N)=O)c[nH]c2c1] | 230 |

### Example 1 Synthesis of Compound 1

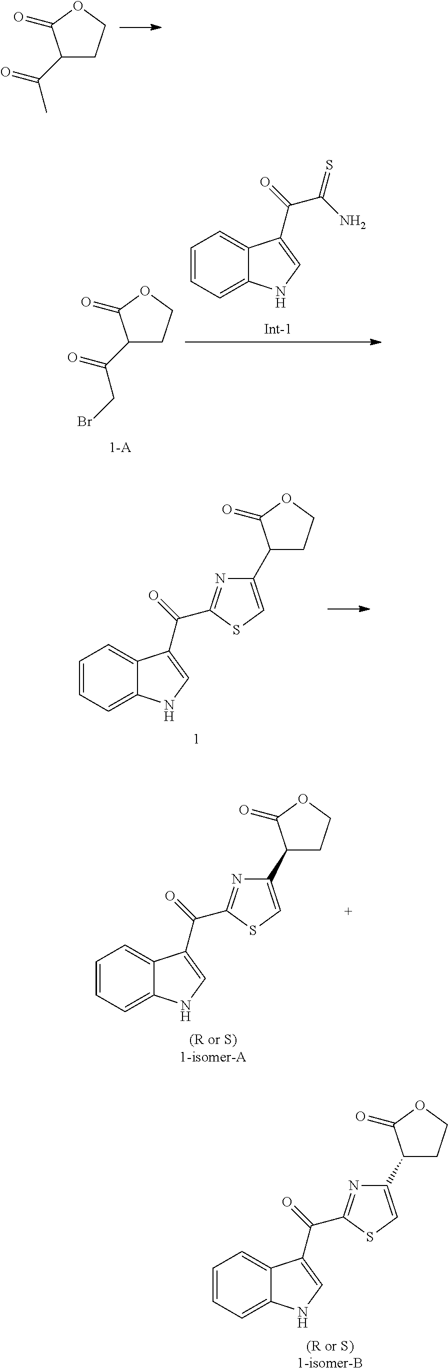

### Step 1: Synthesis of Intermediate 1-A

At room temperature, acetic acid (2.40 g, 40.0 mmol, 1.00 eq.) was added to a solution of 2-acetylbutyrolactone (5.12 g, 40.0 mmol, 1.00 eq.) in dichloromethane (100 mL). The reaction mixture was cooled to below 0° C. A solution of bromine (12.8 g, 80.0 mmol, 2.00 eq.) in dichloromethane (50 mL) was added dropwise over 45 minutes. After the dropwise addition was complete, the reaction mixture was warmed to room temperature and stirred for 1 hour. The reaction mixture was filtered and the filtrate was concentrated under reduced pressure to obtain a brown oily intermediate 1-A (1.50 g, a yield of 18.20%), which was used directly in the next step.

### Step 2: Synthesis of Compound 1

At room temperature, Intermediate 1-A (2.50 g, 12.1 mmol, 1.00 eq.) was added to a solution of intermediate Int-1 (3.00 g, 14.6 mmol, 1.20 eq.) in tetrahydrofuran (30 mL). The mixture was stirred overnight at room temperature in nitrogen atmosphere. The mixture was concentrated to dryness under reduced pressure. Ice water was added, followed by stirring, and a solid precipitated. The solid was filtered to obtain a crude product (2.50 g). The crude product was recrystallized from ethyl acetate to obtain Compound 1 (a yellow solid, 2.30 g, a yield of 51%).

LCMS (ESI): [M+H]₊=313.0.

¹H NMR (400 MHz, DMSO-d₆) δ 12.25 (s, 1H), 8.99 (d, J=2.1 Hz, 1H), 8.38-8.27 (m, 1H), 8.03 (s, 1H), 7.61-7.55 (m, 1H), 7.33-7.25 (m, 2H), 4.58-4.53 (m, 1H), 4.47-4.35 (m, 2H), 2.80-2.60 (m, 2H).

### Compound 1-Isomer-A and Compound 1-Isomer-B

Compound 1 (800 mg) was subjected to chiral preparative separation to obtain Compound 1-isomer-A (340 mg, fraction 1, retention time of 1.163 minutes) and Compound 1-isomer-B (340 mg, fraction 2, retention time of 1.418 minutes).

### Analytical Method:

Instrument: UPCC (waters)
Column: Regis (R, R) Whelk-O1 (4.6×100 mm, 3.5 μm)
Column temperature: 40° C.
Mobile phase: CO₂/MeOH [0.2% (v/v) NH₃ (7 mol/L in MeOH)]=45/55 (v/v)
Flow rate: 3 mL/min
Back pressure: 2000 psi

### Preparation Method:

Instrument: SFC-150 mgm (waters)
Column: Regis (R, R) Whelk-O1 (25×250 mm, 10 μm)
Column temperature: 30° C.
Mobile phase: CO₂/MeOH [0.2% (v/v) NH₃ (7 mol/L in MeOH)]=50/50 (v/v)
Flow rate: 100 mL/min
Back pressure: 100 bar
Detection wavelength: 214 nm

### Compound 1-Isomer-A

LCMS (ESI): [M+H]⁺=313.0.

¹H NMR (400 MHz, MeOH-d₄) δ 9.04 (s, 1H), 8.38-8.35 (m, 1H), 7.82 (s, 1H), 7.52-7.49 (m, 1H), 7.28-7.26 (m, 2H), 4.64-4.59 (m, 1H), 4.50-4.44 (m, 1H), 4.32-4.28 (m, 1H), 2.80-2.60 (in, 2H).

### Compound 1-Isomer-B

LCMS (ESI): [M+H]⁺=313.0.

¹H NMR (400 MHz, MeOH-d₄) δ9.04 (s, 1H), 8.38-8.35 (m, 1H), 7.82 (s, 1H), 7.52-7.49 (m, 1H), 7.28-7.26 (m, 2H), 4.64-4.59 (m, 1H), 4.50-4.44 (m, 1H), 4.32-4.28 (m, 1H), 2.80-2.60 (in, 2H).

TABLE 2
PREPARATION OF COMPOUNDS 1-2 TO 1-9 BY THE SYNTHETIC
METHOD FOR COMPOUND 1

| | Starting | | | |
|---|---|---|---|---|
| Com- | Material | | | |
| pound | A | Starting Material B | Structural Formula | LCMS (ESI) and NMR |
| 1-2 | 1-A | Int-2 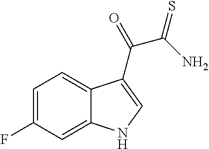 [SMILES: NC(=S)C(=O)c1c[nH]c2cc(F)ccc12] | 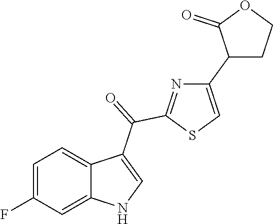 [SMILES: O=C(c1nc(C2CCOC2=O)cs1)c1c[nH]c2cc(F)ccc12] | LCMS (ESI): 331 [M + H]⁺ ¹H NMR (400 MHz, DMSO- d₆) δ 12.27 (d, J = 1.0 Hz, 1H), 8.99 (d, J = 3.2 Hz, 1H), 8.30 (dd, J = 8.7, 5.6 Hz, 1H), 8.05 (s, 1H), 7.39 (dd, J = 9.6, 2.3 Hz, 1H), 7.15 (dt, J = 1.4 Hz, 9.6 Hz, 1H), 4.56 (dt, J = 3.3 Hz, 8.4 Hz, 1H), 4.47-4.33 (m, 2H), 2.83-2.57 (m, 2H). |
| 1-3 | 1-A | Int-3 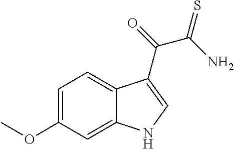 | 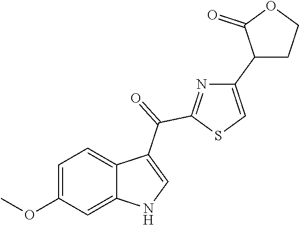 [SMILES: COc1ccc2c(C(=O)c3nc(C4CCOC4=O)cs3)c[nH]c2c1] | LCMS (ESI): 343 [M + H]⁺ ¹H NMR (400 MHz, DMSO- d₆) δ 12.04 (s, 1H), 8.87 (d, J = 3.1 Hz, 1H), 8.16 (d, J = 8.7 Hz, 1H), 8.01 (s, 1H), 7.06 (d, J = 2.1 Hz, 1H), 6.91 (dd, J = 8.7, 2.2 Hz, 1H), 4.55 (td, J = 8.4, 3.3 Hz, 1H), 4.44-4.35 (m, 2H), 3.81 (s, 3H), 2.79- 2.58 (m, 2H). |
| 1-4 | 1-A | Int-4 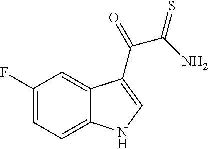 [SMILES: NC(=S)C(=O)c1c[nH]c2ccc(F)cc12] | 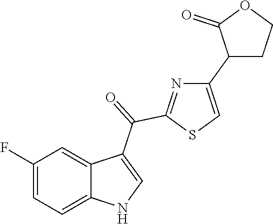 [SMILES: O=C(c1nc(C2CCOC2=O)cs1)c1c[nH]c2ccc(F)cc12] | LCMS (ESI): 331 [M + H]⁺ ¹H NMR (400 MHz, DMSO- d₆) δ 12.35 (s, 1H), 9.03 (d, J = 3.3 Hz, 1H), 8.05 (s, 1H), 7.98 (dd, J = 9.8, 2.6 Hz, 1H), 7.60 (dd, J = 8.9, 4.6 Hz, 1H), 7.16 (td, J = 9.2, 2.6 Hz, 1H), 4.56 (td, J = 8.4, 3.3 Hz, 1H), 4.46-4.32 (m, 2H), 2.79-2.61 (m, 2H). |
| 1-5 | 1-A | Int-5 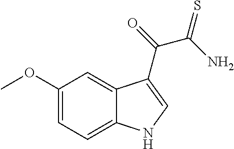 | 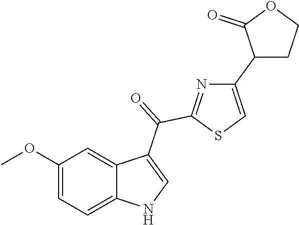 [SMILES: COc1ccc2[nH]cc(C(=O)c3nc(C4CCOC4=O)cs3)c2c1] | LCMS (ESI): 343 [M + H]⁺ ¹H NMR (400 MHz, DMSO- d₆) δ 12.15 (s, 1H), 8.93 (d, J = 1.0 Hz, 1H), 8.02 (s, 1H), 7.84 (s, 1H), 7.47 (d, J = 8.7 Hz, 1H), 6.92 (d, J = 6.8 Hz, 1H), 4.57-4.53 (m, 1H), 4.47- 4.27 (m, 2H), 3.82 (s, 3H), 2.84-2.58 (m, 2H). |
| 1-6 | 1-A | Int-6 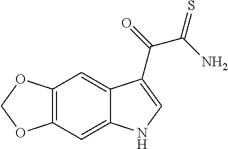 [SMILES: NC(=S)C(=O)c1c[nH]c2cc3c(cc12)OCO3] | 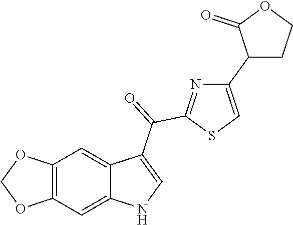 [SMILES: O=C(c1nc(C2CCOC2=O)cs1)c1c[nH]c2cc3c(cc12)OCO3] | LCMS (ESI): 357 [M + H]⁺ ¹H NMR (400 MHz, DMSO- d₆) δ 12.05 (d, J = 1.0 Hz, 1H), 8.80 (d, J = 1.0 Hz, 1H), 8.01 (s, 1H), 7.70 (s, 1H), 7.10 (s, 1H), 6.04 (s, 2H), 4.55 (td, J = 8.4, 1.0 Hz, 1H), 4.46-4.26 (m, 2H), 2.91-2.55 (m, 2H). |
| 1-7 | 1-A | Int-7 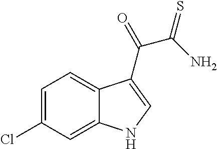 [SMILES: NC(=S)C(=O)c1c[nH]c2cc(Cl)ccc12] | 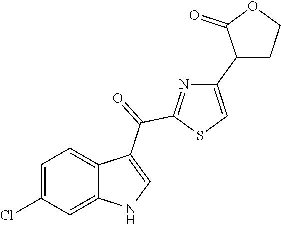 [SMILES: O=C(c1nc(C2CCOC2=O)cs1)c1c[nH]c2cc(Cl)ccc12] | LCMS (ESI): 347 [M + H]⁺ ¹H NMR (400 MHz, DMSO- d₆) δ 12.04 (s, 1H), 8.99 (d, J = 1.0 Hz, 1H), 8.36 (d, J = 8.7 Hz, 1H), 8.00 (s, 1H), 7.36 (d, J = 2.1 Hz, 1H), 7.20 (dd, J = 8.7, 2.2 Hz, 1H), 4.55 (td, J = 8.4, 3.3 Hz, 1H), 4.43-4.35 (m, 2H), 2.79-2.58 (m, 2H). |
| 1-8 | 1-A | Int-8 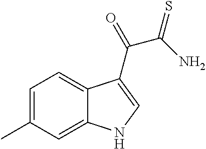 | 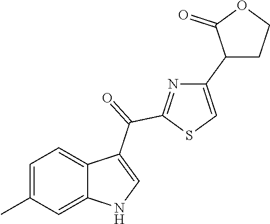 [SMILES: Cc1ccc2c(C(=O)c3nc(C4CCOC4=O)cs3)c[nH]c2c1] | LCMS (ESI): 327 [M + H]⁺ ¹H NMR (400 MHz, DMSO- d₆) δ 12.04 (s, 1H), 8.87 (d, J = 3.1 Hz, 1H), 8.16 (d, J = 8.7 Hz, 1H), 8.01 (s, 1H), 7.06 (d, J = 2.1 Hz, 1H), 6.91 (dd, J = 8.7, 2.2 Hz, 1H), 4.55 (td, J = 8.4, 3.3 Hz, 1H), 4.44-4.35 (m, 2H), 2.79-2.58 (m, 2H). 2.11 (s, 3H). |
| 1-9 | 1-A | Int-9 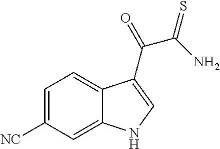 [SMILES: N#Cc1ccc2c(C(=O)C(N)=S)c[nH]c2c1] | 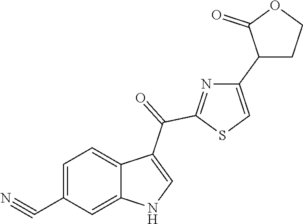 [SMILES: N#Cc1ccc2c(C(=O)c3nc(C4CCOC4=O)cs3)c[nH]c2c1] | LCMS (ESI): 338 [M + H]⁺. |

### Example 2 Synthesis of Compound 2

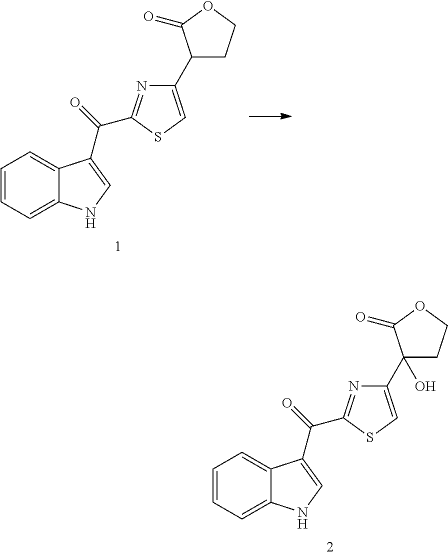 [SMILES: O=C(c1nc(C2CCOC2=O)cs1)c1c[nH]c2ccccc12 ; O=C(c1nc(C2(O)CCOC2=O)cs1)c1c[nH]c2ccccc12]

At room temperature, 1,5,7-Triazabicyclo [4.4.0]dec-5-ene (TBD, 5.00 mg, 0.032 mmol, 0.10 eq.) was added to a solution of Compound 1 (0.10 g, 0.32 mmol, 1.00 eq.) in N, N-dimethylformamide (DMF, 5 mL). The solution was warmed to 45° C. and stirred overnight with the reaction vessel open. The reaction mixture was concentrated to dryness under reduced pressure, followed by purification by silica gel column chromatography (ethyl acetate: petroleum ether=0% to 50%, i.e., a volume percentage of ethyl acetate in the mixed mobile phase of ethyl acetate and petroleum ether was 0%-50%) to obtain Compound 2 (a yellow solid, 70 mg, a yield of 700%).

LCMS (ESI): [M+H]⁺=329.0.

¹H NMR (400 MHz, DMSO-d₆) δ 12.26 (s, 1H), 8.93 (d, J=3.3 Hz, 1H), 8.31 (dd, J=5.9, 3.2 Hz, 1H), 8.09 (s, 1H), 7.68-7.50 (m, 1H), 7.40-7.20 (m, 2H), 6.98 (s, 1H), 4.64-4.43 (m, 2H), 2.94-2.88 (m, 1H), 2.62-2.52 (m, 1H).

TABLE 3
PREPARATION OF COMPOUNDS 2-2 TO 2-5 BY THE SYNTHETIC
METHOD FOR COMPOUND 2 IN EXAMPLE 2

| | Starting | | |
|---|---|---|---|
| Compound | Material | Structural Formula | LCMS (ESI): [M + H]+ |
| 2-2 | Compound 1-2 | 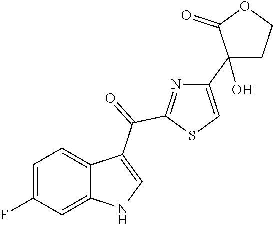 [SMILES: O=C(c1nc(C2(O)CCOC2=O)cs1)c1c[nH]c2cc(F)ccc12] | LCMS (ESI): 347 [M + H]⁺ ¹H NMR (400 MHz, DMSO-d₆) δ 12.29 (d, J = 1.0 Hz, 1H), 8.99 (d, J = 3.2 Hz, 1H), 8.31 (dd, J = 8.7, 5.6 Hz, 1H), 810 (s, 1H), 7.39 (dd, J = 9.6, 2.3 Hz, 1H), 7.15 (dt, J = 1.4 Hz, 9.6 Hz, 1H), 6.99 (s, 1H), 4.64-4.43 (m, 2H), 2.94-2.88 (m, 1H), 2.62-2.52 (m, 1H) |
| 2-3 | Compound 1-3 | 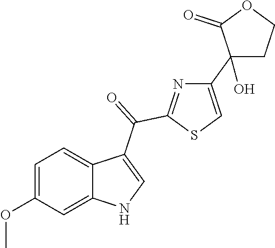 [SMILES: COc1ccc2c(C(=O)c3nc(C4(O)CCOC4=O)cs3)c[nH]c2c1] | LCMS (ESI): 359 [M + H]⁺ |
| 2-4 | Compound 1-4 | 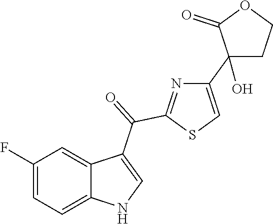 [SMILES: O=C(c1nc(C2(O)CCOC2=O)cs1)c1c[nH]c2ccc(F)cc12] | LCMS (ESI): 347 [M + H]⁺ |
| 2-5 | Compound 1-5 | 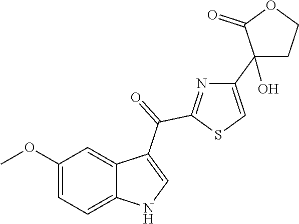 [SMILES: COc1ccc2[nH]cc(C(=O)c3nc(C4(O)CCOC4=O)cs3)c2c1] | LCMS (ESI): 359 [M + H]⁺ |

### Example 3 Synthesis of Compound 3

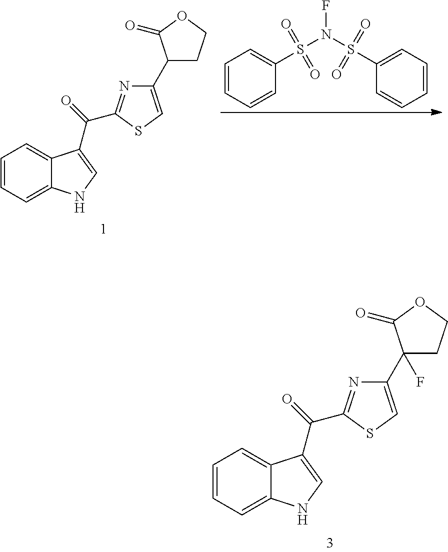

In nitrogen atmosphere, NaH (60.0 mg, 2.50 mmol, 2.50 eq.) was added to a solution of Compound 1 (0.312 g, 1.00 mmol, 1.00 eq.) in tetrahydrofuran (5 mL) at room temperature. After stirring for half an hour, N-fluorobenzenesulfonimide (378 mg, 1.20 mmol, 1.20 eq.) was added, and the mixture was stirred at room temperature for 1 hour. The mixture was concentrated to dryness under reduced pressure and purified by silica gel column chromatography (methanol:dichloromethane=0% to 100%, i.e., a volume percentage of methanol in the mixed mobile phase of methanol and dichloromethane was 0%-100%) to obtain Compound 3 (a yellow solid, 40 mg, a yield of 10%).

LCMS (ESI): [M+H]⁺=331.0.

¹H NMR (400 MHz, DMSO-d₆) δ 12.31 (s, 1H), 8.97 (s, 1H), 8.41 (s, 1H), 8.34-8.11 (m, 1H), 7.59-7.56 (m, 1H), 7.41-7.08 (m, 2H), 4.70-4.64 (m, 1H), 4.62-4.41 (m, 1H), 3.29-3.19 (m, 1H), 3.10-2.93 (m, 1H).

TABLE 4
PREPARATION OF COMPOUNDS 3-2 TO 3-5 BY THE SYNTHETIC
METHOD FOR COMPOUND 3 IN EXAMPLE 3

| | Starting | | |
|---|---|---|---|
| Compound | Material | Structural Formula | LCMS (ESI): [M + H]⁺ |
| 3-2 | Compound 1-2 | 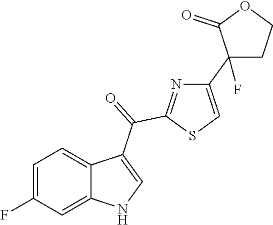 [SMILES: O=C(c1nc(C2(F)CCOC2=O)cs1)c1c[nH]c2cc(F)ccc12] | LCMS (ESI): 349 [M + H]⁺ |
| 3-3 | Compound 1-3 | 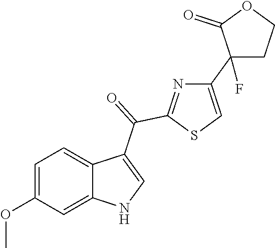 [SMILES: COc1ccc2c(C(=O)c3nc(C4(F)CCOC4=O)cs3)c[nH]c2c1] | LCMS (ESI): 361 [M + H]⁺ |
| 3-4 | Compound 1-4 | 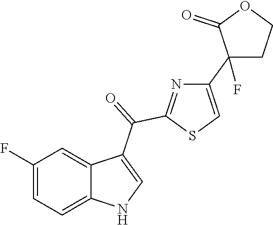 [SMILES: O=C(c1nc(C2(F)CCOC2=O)cs1)c1c[nH]c2ccc(F)cc12] | LCMS (ESI): 349 [M + H]⁺ |
| 3-5 | Compound 1-5 | 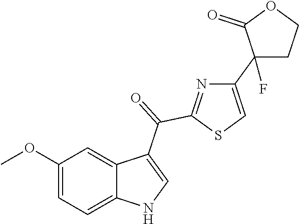 [SMILES: COc1ccc2[nH]cc(C(=O)c3nc(C4(F)CCOC4=O)cs3)c2c1] | LCMS (ESI): 361 [M + H]⁺ |

### Example 4 Synthesis of Compound 4

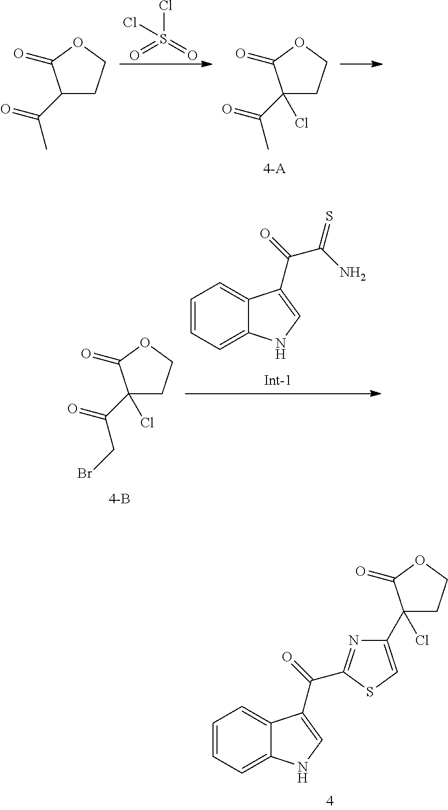

### Step 1: Synthesis of Intermediate 4-A

In nitrogen atmosphere at 0° C., sulfuryl chloride (5.49 g, 41.0 mmol, 1.05 eq.) was added dropwise to alpha-acetyl-gamma-butyrolactone (5.00 g, 39.0 mmol, 1.00 eq.). The reaction mixture was stirred at room temperature for 5 hours. The mixture was poured into water (100 mL) and extracted three times with ethyl acetate (50 mL×3), wherein a volume of the ethyl acetate used each time was 50 mL. The combined organic phases were washed with saturated brine, dried over anhydrous sodium sulfate, filtered, and concentrated under reduced pressure to obtain brown oily intermediate 4-A (6.00 g, a yield of 95%), which was used directly in the next step without further purification.

### Step 2: Synthesis of Intermediate 4-B

In nitrogen atmosphere at 0° C., a solution of bromine (9.88 g, 61.7 mmol, 2.00 eq.) in dichloromethane (30 mL) was added dropwise to a solution of intermediate 4-A (5.00 g, 30.9 mmol, 1.00 eq.) in dichloromethane (20 mL). The reaction mixture was stirred at room temperature overnight. The mixture was concentrated under reduced pressure to obtain brown oily intermediate 4-B (6.70 g, a yield of 90%), which was used directly in the next step without further purification.

### Step 3: Synthesis of Compound 4

In nitrogen atmosphere at room temperature, Intermediate Int-1 (2.1 g, 10.3 mmol, 1.03 eq.) was added to a solution of intermediate 4-B (2.41 g, 10.0 mmol, 1.00 eq.) in tetrahydrofuran (20 mL). The reaction mixture was stirred at room temperature overnight. The mixture was poured into ice water (100 mL) and extracted three times with ethyl acetate (50 mL×3), wherein a volume of the ethyl acetate used each time was 50 mL. The combined organic phases were washed with saturated brine, dried over anhydrous sodium sulfate, filtered, and concentrated to dryness under reduced pressure. The crude product was purified by silica gel column chromatography (methanol:dichloromethane=0% to 30%, i.e., a volume percentage of methanol in the mixed mobile phase of methanol and dichloromethane was 0%-30%) to obtain Compound 4 (a yellow solid, 2.3 g, a yield of 67%).

LCMS (ESI): 347 [M+H]⁺.

TABLE 5
PREPARATION OF COMPOUNDS 4-2 TO 4-5 BY THE SYNTHETIC
METHOD FOR COMPOUND 4 IN EXAMPLE 4

| | Starting | | |
|---|---|---|---|
| Compound | Material | Structural Formula | LCMS (ESI): [M + H]⁺ |
| 4-2 | Int-2 | 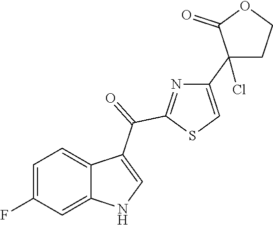 [SMILES: O=C(c1nc(C2(Cl)CCOC2=O)cs1)c1c[nH]c2cc(F)ccc12] | LCMS (ESI): 365 [M + H]⁺ |
| 4-3 | Int-3 | 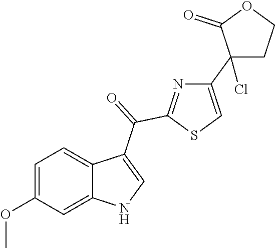 [SMILES: COc1ccc2c(C(=O)c3nc(C4(Cl)CCOC4=O)cs3)c[nH]c2c1] | LCMS (ESI): 377 [M + H]⁺ |
| 4-4 | Int-4 | 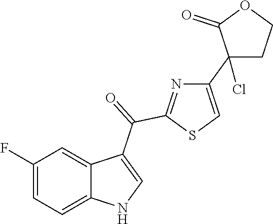 [SMILES: O=C(c1nc(C2(Cl)CCOC2=O)cs1)c1c[nH]c2ccc(F)cc12] | LCMS (ESI): 365 [M + H]⁺ |
| 4-5 | Int-5 | 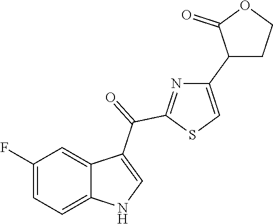 [SMILES: O=C(c1nc(C2CCOC2=O)cs1)c1c[nH]c2ccc(F)cc12] | LCMS (ESI): 377 [M + H]⁺ |

### Example 5 Synthesis of Compound 5

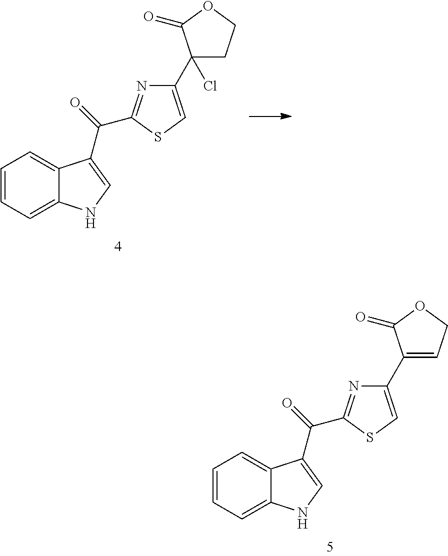 [SMILES: O=C(c1nc(C2(Cl)CCOC2=O)cs1)c1c[nH]c2ccccc12 ; O=C1OCC=C1c1csc(C(=O)c2c[nH]c3ccccc23)n1]

In nitrogen atmosphere at room temperature, triethylamine (58.5 mg, 0.578 mmol, 2.00 eq.) and triethylamine trihydrofluoride (93.2 mg, 0.578 mmol, 2.00 eq.) were added to a solution of compound 4 (0.100 g, 0.289 mmol, 1.00 eq.) in acetonitrile (3 mL). The mixture was stirred under reflux overnight. After cooling to room temperature, the mixture was concentrated to dryness under reduced pressure and purified by silica gel column chromatography (methanol:dichloromethane=0% to 30%, i.e., a volume percentage of methanol in the mixed mobile phase of methanol and dichloromethane was 0%-30%) to obtain Compound 5 (a yellow solid, 0.01 g, a yield of 11%).

LCMS (ESI): [M+H]⁺=311.2. ¹H NMR (400 MHz, DMSO-d₆) δ 12.30 (s, 1H), 9.27 (s, 1H), 8.51 (s, 2H), 8.33 (dd, J=5.8, 3.2 Hz, 1H), 7.59 (dd, J=5.8, 2.8 Hz, 1H), 7.37-7.16 (m, 2H), 5.17 (d, J=1.3 Hz, 2H).

### Example 6

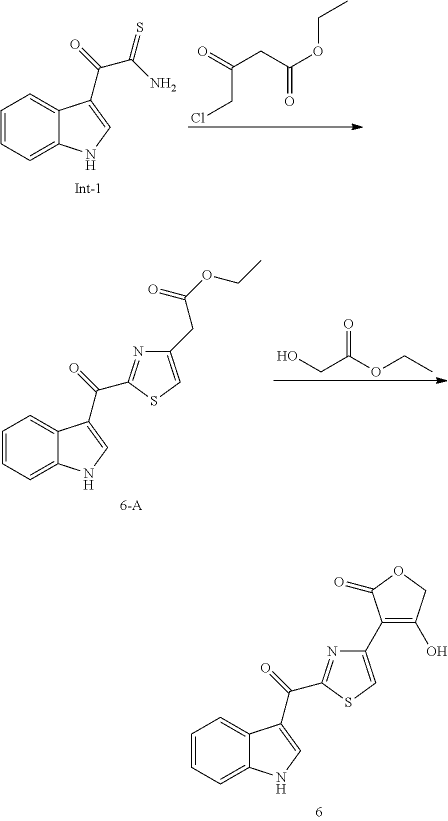

### Step 1: Synthesis of Intermediate 6-A

Intermediate 6-A was prepared by the same procedure as Step 2 for the synthesis of Compound 1, and the yield was 78%.

### Step 2: Synthesis of Compound 6

In nitrogen atmosphere at room temperature, ethyl glycolate (0.13 g, 1.27 mmol, 4.00 eq.) and potassium tert-butoxide (0.29 g, 2.55 mmol, 8.00 eq.) were added to a solution of intermediate 6-A (0.10 g, 0.32 mmol, 1.00 eq.) in tetrahydrofuran (5 mL). The mixture was stirred at room temperature overnight. The mixture was poured into ice water (10 mL) and the pH was adjusted to 7 with a saturated aqueous tartaric acid solution. The mixture was extracted three times with ethyl acetate (10 mL×3), and a volume of the ethyl acetate used each time was 10 mL. The combined organic phases were washed with saturated brine, dried over anhydrous sodium sulfate, filtered, and concentrated to dryness under reduced pressure. The crude product was purified by preparative high-performance liquid chromatography (HPLC) to obtain Compound 6 (a yellow solid, 45.00 mg, 43%).

LCMS [M+H]⁺=327.0.

¹H NMR (400 MHz, MeOH-d4) δ 9.50 (s, 1H), 8.39-8.37 (m, 1H), 7.96 (s, 1H), 7.53-7.44 (m, 1H), 7.30-7.19 (m, 2H), 4.54 (s, 2H).

### Example 7

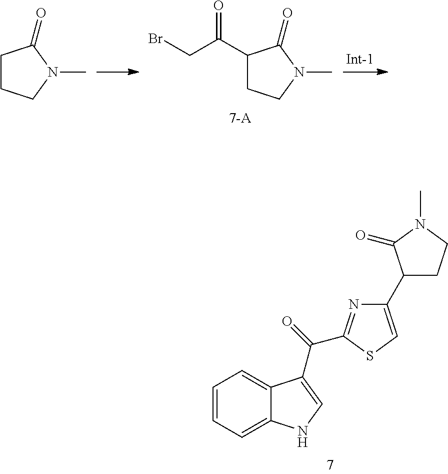

### Step 1: Synthesis of Intermediate 7-A

A freshly prepared solution of lithium diisopropylamide (LDA), which was prepare by adding dropwise n-Butyllithium (n-BuLi) (4.04 mL, 10.09 mmol, 2.5 mol/L in tetrahydrofuran (THF)) to a solution of diisopropylamine (1.42 mL, 10.09 mmol) in anhydrous THF (20 mL) at −78° C., was added dropwise to a solution of N-methyl-2-pyrrolidinone (1.0 g, 10.09 mmol) in anhydrous THF (15.0 mL) at −78° C. The mixture was stirred at the same temperature for 1 hour. A solution of ethyl bromoacetate (1.34 mL, 12.11 mmol) was added dropwise at −78° C. over 5 minutes. The mixture was stirred at the same temperature for 3 hours. The temperature was slowly raised to −20° C. 1 mol/L HCl (20 mL) was added dropwise while controlling the temperature not to exceed −20° C. The mixture was stirred for 10 to 15 minutes. The reaction mixture was diluted with ice water (30-40 g), and the layers were separated. The aqueous layer was extracted three times with ethyl acetate (EtOAc) (3×30 mL), and a volume of EtOAc used each time was 30 mL. The combined ethyl acetate phases were dried over Na₂SO₄ and concentrated under reduced pressure. The resulting crude residue was purified by column chromatography to obtain compound 7-A (a pale yellow oil, 1.230 g, a yield of 69%).

LCMS [M+H]⁺=220, 222.

### Step 2: Synthesis of Compound 7

The Compound 7 was prepared by the same procedure as Step 2 for the Synthesis of Compound 1. Intermediate 7-A (330 mg, 1.5 mmol) was added to a solution of intermediate Int-1 (300 mg, 1.46 mmol, 1.20 eq.) in tetrahydrofuran (10 mL) at room temperature. The mixture was stirred at room temperature in nitrogen atmosphere overnight. The mixture was concentrated to dryness under reduced pressure. Ice water was added, followed by stirring, and a solid was precipitated. The solid was filtered to obtain a crude product (152 mg). The crude product was recrystallized from ethyl acetate to obtain Compound 7 (a yellow solid, 120 mg, a yield of 54%).

LCMS (ESI): [M+H]⁺=326.1.

¹H NMR (400 MHz, DMSO-d₆) δ 12.25 (s, 1H), 8.99 (d, J=3.2 Hz, 1H), 8.36-8.25 (m, 1H), 7.90 (s, 1H), 7.66-7.49 (m, 1H), 7.38-7.16 (m, 2H), 3.99 (t, J=8.7 Hz, 1H), 3.61-3.41 (m, 2H), 2.84 (s, 3H), 2.55-2.49 (m, 1H), 2.38-2.33 (m, 1H).

TABLE 6
PREPARATION OF COMPOUNDS 7-2 TO 7-6 BY THE SYNTHETIC
METHOD FOR COMPOUND 7 IN EXAMPLE 7

| | Starting | | | |
|---|---|---|---|---|
| Com- | Material | | | |
| pound | A | Starting Material B | Structural Formula | LCMS and NMR |
| 7-2 | 7-A | Int-2 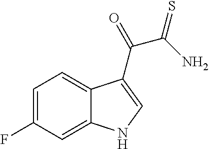 [SMILES: NC(=S)C(=O)c1c[nH]c2cc(F)ccc12] | 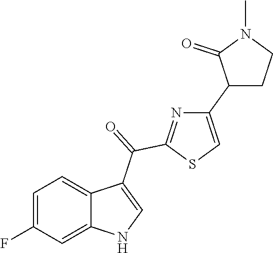 [SMILES: CN1CCC(c2csc(C(=O)c3c[nH]c4cc(F)ccc34)n2)C1=O] | LCMS (ESI): 344.1 [M + H]⁺ ¹H NMR (400 MHz, DMSO- d₆) δ 12.27 (s, 1H), 8.98 (s, 1H), 8.29 (dd, J = 8.8, 5.6 Hz, 1H), 7.92 (s, 1H), 7.38 (dd, J = 9.6, 2.3 Hz, 1H), 7.14 (td, J = 9.6, 2.4 Hz, 1H), 3.99 (t, J = 8.7 Hz, 1H), 3.64-3.40 (m, 2H), 2.84 (s, 3H), 2.60-2.49 (m, 1H), 2.38-2.33 (m, 1H). |
| 7-3 | 7-A | Int-3 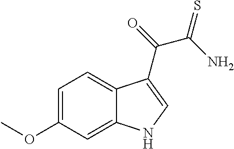 | 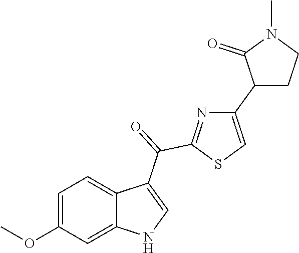 [SMILES: COc1ccc2c(C(=O)c3nc(C4CCN(C)C4=O)cs3)c[nH]c2c1] | LCMS (ESI): 356.1 [M + H]⁺ ¹H NMR (400 MHz, DMSO- d₆) δ 12.04 (s, 1H), 8.86 (s, 1H), 8.16 (d, J = 8.7 Hz, 1H), 7.88 (s, 1H), 7.06 (d, J = 2.2 Hz, 1H), 6.91 (dd, J = 8.7, 2.3 Hz, 1H), 3.97 (t, J = 8.7 Hz, 1H), 3.81 (s, 3H), 3.61-3.37 (m, 2H), 2.83 (s, 3H), 2.56- 2.49 (m, 1H), 2.47-2.33 (m, 1H). |
| 7-4 | 7-A | Int-4 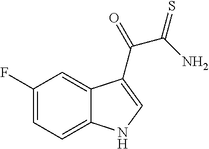 [SMILES: NC(=S)C(=O)c1c[nH]c2ccc(F)cc12] | 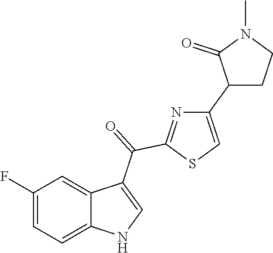 [SMILES: CN1CCC(c2csc(C(=O)c3c[nH]c4ccc(F)cc34)n2)C1=O] | LCMS (ESI): 344.1 [M + H]⁺ ¹H NMR (400 MHz, DMSO- d₆) δ 12.35 (s, 1H), 9.02 (d, J = 3.3 Hz, 1H), 7.98 (dd, J = 9.9, 2.6 Hz, 1H), 7.92 (s, 1H), 7.59 (dd, J = 8.8, 4.6 Hz, 1H), 7.15 (td, J = 9.2, 2.6 Hz, 1H), 3.99 (t, J = 8.7 Hz, 1H), 3.62- 3.41 (m, 2H), 2.83 (s, 3H), 2.58-2.49 (m, 1H), 2.38-2.33 (m, 1H). |
| 7-5 | 7-A | Int-5 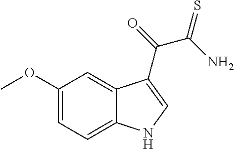 | 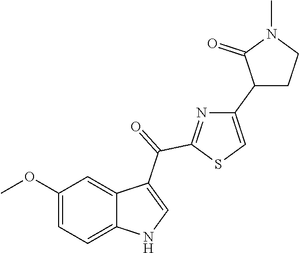 [SMILES: COc1ccc2[nH]cc(C(=O)c3nc(C4CCN(C)C4=O)cs3)c2c1] | LCMS (ESI): 356.1 [M + H]⁺ ¹H NMR (400 MHz, DMSO- d₆) δ 12.15 (d, J = 2.4 Hz, 1H), 8.93 (d, J = 3.3 Hz, 1H), 7.89 (s, 1H), 7.84 (d, J = 2.5 Hz, 1H), 7.46 (d, J = 8.8 Hz, 1H), 6.91 (dd, J = 8.8, 2.6 Hz, 1H), 3.98 (t, J = 8.7 Hz, 1H), 3.82 (s, 3H), 3.56-3.38 (m, 2H), 2.84 (s, 3H), 2.59-2.47 (m, 1H), 2.40-2.33 (m, 1H). |
| 7-6 | 7-A | Int-6 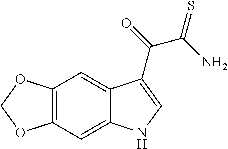 [SMILES: NC(=S)C(=O)c1c[nH]c2cc3c(cc12)OCO3] | 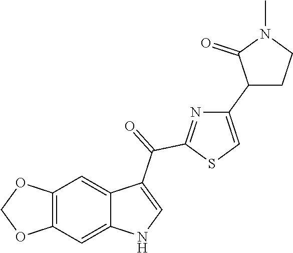 [SMILES: CN1CCC(c2csc(C(=O)c3c[nH]c4cc5c(cc34)OCO5)n2)C1=O] | LCMS (ESI): 370.1 [M + H]⁺ ¹H NMR (400 MHz, DMSO- d₆) δ 12.05 (s, 1H), 8.79 (s, 1H), 7.88 (s, 1H), 7.70 (s, 1H), 7.10 (s, 1H), 6.04 (s, 2H), 3.97 (t, J = 8.7 Hz, 1H), 3.62-3.40 (m, 2H), 2.83 (s, 3H), 2.58- 2.47 (m, 1H), 2.36-2.31 (m, 1H). |

### Example 8

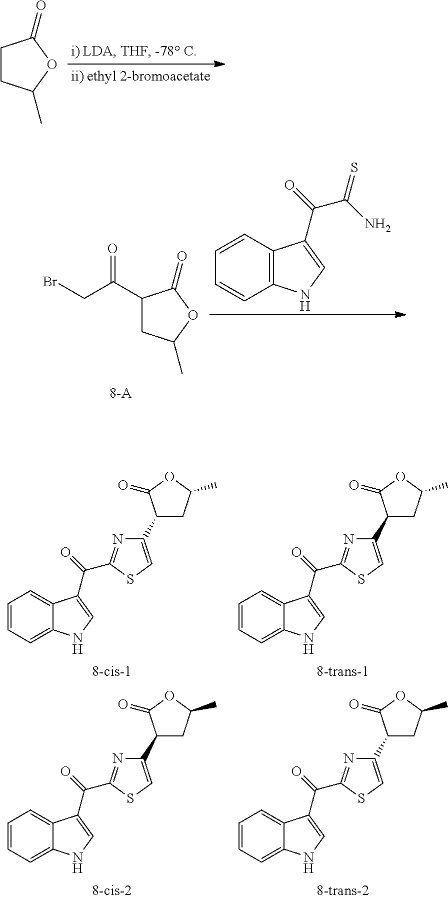

### Step 1: Synthesis of Intermediate 8-A

A freshly prepared solution of LDA, which was prepared by adding dropwise n-BuLi (4.04 mL, 10.09 mmol, 2.5 mol/L in THF to a solution of diisopropylamine (1.42 mL, 10.09 mmol) in anhydrous THF (20 mL) at −78° C., was slowly added dropwise to a solution of γ-valerolactone (1.1 g, 11 mmol) in anhydrous THF (15.0 mL) at −78° C. The mixture was stirred at the same temperature for 1 hour. At −78° C., a solution of ethyl bromoacetate (1.34 mL, 12.11 mmol) was added dropwise over 5 minutes. The mixture was stirred at the same temperature for 3 hours. The mixture was slowly warmed to −20° C. 1 mol/L hydrochloric acid (HCl) (20 mL) was added dropwise while controlling the temperature not to exceed −20° C. The mixture was stirred for 10 to 15 minutes. The reaction mixture was diluted with ice water (30 g-40 g) and the layers were separated. The aqueous layer was extracted three times with EtOAc (3×30 mL), and a volume of EtOAc used each time was 30 mL. The combined ethyl acetate phases were dried over sodium sulfate (Na₂SO₄) and concentrated under reduced pressure. The resulting crude residue was purified by column chromatography to obtain compound 8-A (a pale yellow oil, 1.230 g, a yield of 69%).

LCMS [M+H]⁺=221, 223.

### Step 2: Synthesis of Compound 8

The Compound 8 was prepared by the same procedure as Step 2 for synthesis of compound 1. At room temperature, intermediate 8-A (332 mg, 1.5 mmol) was added to a solution of intermediate Int-1 (300 mg, 1.46 mmol, 1.20 eq.) in THF (10 mL). The mixture was stirred overnight at room temperature in nitrogen atmosphere. The mixture was concentrated to dryness under reduced pressure. Ice water was added and the mixture was stirred, resulting in precipitation of a solid. The solid was filtered to obtain a crude product (152 mg). The crude product was recrystallized from ethyl acetate to obtain Compound 8 (a yellow solid, 343 mg, a yield of 72%).

LCMS (ESI): [M+H]⁺=327.1.

¹H NMR (400 MHz, DMSO-d₆) δ 12.29 (s, 1H), 9.00 (d, J=3.2 Hz, 0.6H), 8.99 (d, J=3.2 Hz, 0.4H), 8.31-8.33 (m, 1H), 8.02 (s, 0.6H), 8.01 (s, 0.4H), 7.55-7.58 (m, 1H), 7.25-7.32 (m, 2H), 4.95-5.00 (m, 0.4H), 4.72-4.77 (m, 0.6H), 4.48-4.54 (m, 1H), 2.83-2.90 (m, 0.6H), 2.72-2.79 (m, 0.4H), 2.32-2.44 (m, 0.4H), 2.24-2.32 (m, 0.6H), 1.48 (d, J=8.0 Hz, 1.8H), 1.44 (d, J=8.0 Hz, 1.2H).

### Step 3: Isomer Resolution

Compound 8 obtained from Step 2 was subjected to chromatographic separation. The resolution conditions were as follows:
Mobile Phase (Co_Solvent): MeOH [0.2% (v/v) NH₃ (7 mol/L in MeOH)]
Column (Column Name): AD-3 4.6*100 mm 3 um
Back Pressure: 2000 psi
Flow rate: 3.0 mL/min
Detector (Channel Name): PDA Ch1 MaxPlot (210-400 nm)
Detection Wavelength (Proc. Chnl. Descr.): PDA Ch1 MaxPlot (210-400 nm)
Column Temperature: 40° C.
Run Time: 6.0 minutes

Four diastereomers were obtained after separation, which were 8-cis-1, 8-cis-2, 8-trans-1, and 8-trans-2.

TABLE 7
PREPARATION OF COMPOUNDS 8-2 TO 8-6 BY THE SYNTHETIC
METHOD FOR COMPOUND 8 IN EXAMPLE 8

| | Starting | | | |
|---|---|---|---|---|
| Com- | Material | | | |
| pound | A | Starting Material B | Structural Formula | LCMS and NMR |
| 8-2 | 8-A | Int-2 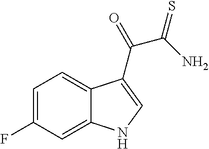 [SMILES: NC(=S)C(=O)c1c[nH]c2cc(F)ccc12] | 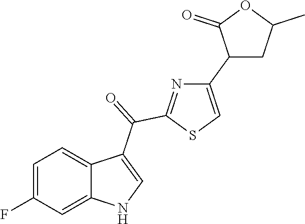 [SMILES: CC1CC(c2csc(C(=O)c3c[nH]c4cc(F)ccc34)n2)C(=O)O1] | LCMS (ESI): 345.1 [M + H]⁺ ¹H NMR (400 MHz, DMSO-d₆) δ 12.30 (s, 1H), 8.97 + 8.98 (two s, 1H), 8.30 + 8.28 (two d, J = 8.0 Hz, 1H), 8.04 + 8.02 (two s, 1H), 7.40 + 7.38 (two d, 1H), 7.12-7.17 (m, 1H), 4.7-4.97 (m, 0.55H), 4.72-4.76 (m, 0.45H), −7.58 (m, 1H), 7.25-7.32 (m, 2H), 4.95-5.00 (m, 0.4H), 4.72- 4.77 (m, 0.6H), 4.48-4.54 (m, 1H), 2.83-2.90 (m, 0.6H), 2.72- 2.79 (m, 0.4H), 2.32-2.44 (m, 0.4H), 2.24-2.32 (m, 0.6H), |
| | | | | 1.48 (d, J = 8.0 Hz, 1.65H), 1.44 |
| | | | | (d, J = 8.0 Hz, 1.35H) |
| 8-3 | 8-A | Int-3 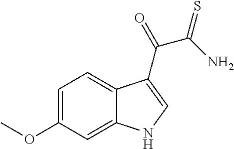 | 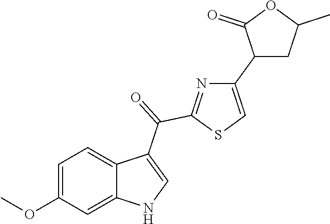 | LCMS (ESI): 357.1 [M + H]⁺ ¹H NMR (400 MHz, DMSO-d₆) δ 12.29 (s, 1H), 8.88 + 8.86 (two d, 1H), 8.16 (d, J = 8.0 Hz, 1H), 8.01 + 7.99 (two s, 1H), 7.05 + 7.06 (two s, 1H), 6.90-6.92 (two d, 1H), 4.95-5.01 (m, 0.45H), 4.72-4.78 (m, 0.55H), 4.45-4.3 (m, 1H), 3.81 (s, 3H), 2.83-2.90 (m, 0.55H), 2.70-2.78 (m, 0.45H), 2.35-2.44 (m, 0.45H), 2.22-2.31 (m, 0.55H), 1.48 (d, J = 8.0 Hz, 1.65H), 1.44 (d, J = 8.0 Hz, 1.35H) |
| 8-4 | 8-A | Int-4 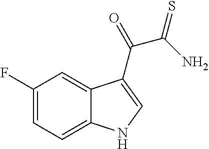 [SMILES: NC(=S)C(=O)c1c[nH]c2ccc(F)cc12] | 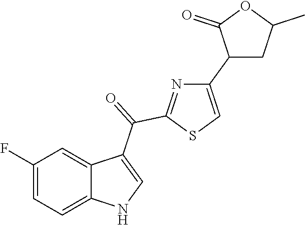 [SMILES: CC1CC(c2csc(C(=O)c3c[nH]c4ccc(F)cc34)n2)C(=O)O1] | LCMS (ESI): 345.1 [M + H]⁺ ¹H NMR (400 MHz, DMSO-d₆) δ 12.38 (s, 1H), 9.02 + 9.04 (two s, 1H), 8.04-7.97 (m, 2H), 7.98 + 7.99 (two s, 1H), 7.13-7.18 (m, 1H), 4.95-5.00 (m, 0.45H), 4.72- 4.77 (m, 0.55H), 4.48-4.53 (m, 1H), 2.74-2.88 (two m, 1H), 2.31- 2.47 (m, 0.45H), 2.19-2.31 (m, 0.55H), 1.48 (d, J = 8.0 Hz, 1.65H), 1.44 (d, J = 8.0 Hz, 1.35H) |
| 8-5 | 8-A | Int-5 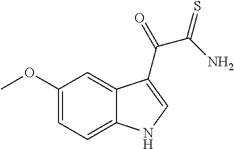 | 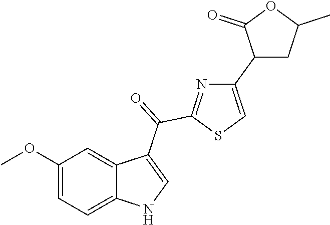 | LCMS (ESI): 356.1 [M + H]⁺ ¹H NMR (400 MHz, DMSO-d₆) δ 12.18 (s, 1H), 8.92 + 8.94 (two d, 1H), 8.01 + 7.99 (two s, 1H), 7.83 + 7.84 (two s, 1H), 7.45 + 7.47 (two s, 1H), 6.90-6.93 (two d, 1H), 4.93-5.01 (m, 0.45H), 4.71-4.75 (m, 0.55H), 4.47-4.53 (m, 1H), 3.82 (s, 3H), 2.83-2.90 (m, 0.55H), 2.72-2.78 (m, 0.45H), 2.37-2.44 (m, 0.45H), 2.22-2.31 (m, 0.55H), 1.48 (d, J = 8.0 Hz, 1.65H), 1.44 (d, J = 8.0 Hz, 1.35H) |
| 8-6 | 8-A | Int-6 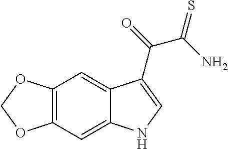 [SMILES: NC(=S)C(=O)c1c[nH]c2cc3c(cc12)OCO3] | 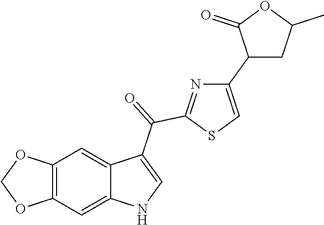 [SMILES: CC1CC(c2csc(C(=O)c3c[nH]c4cc5c(cc34)OCO5)n2)C(=O)O1] | LCMS (ESI): 371.1 [M + H]⁺ ¹H NMR (400 MHz, DMSO-d₆) δ 12.18 (s, 1H), 8.81 + 8.79 (two s, 1H), 8.00 + 7.98 (two s, 1H), 7.70 (two s, 1H), 7.10 (s, 1H), 6.04 (s, 2H), 4.96-4.97 (m, 0.45H), 4.71-4.75 (m, 0.55H), 4.47-4.53 (m, 1H), 2.85-2.88 (m, 0.55H), 2.72-2.75 (m, 0.45H), 2.37-2.44 (m, 0.45H), 2.22-2.31 (m, 0.55H), 1.47 (d, J = 8.0 Hz, 1.65H), 1.43 (d, J = 8.0 Hz, 1.35H) |

### Example 9

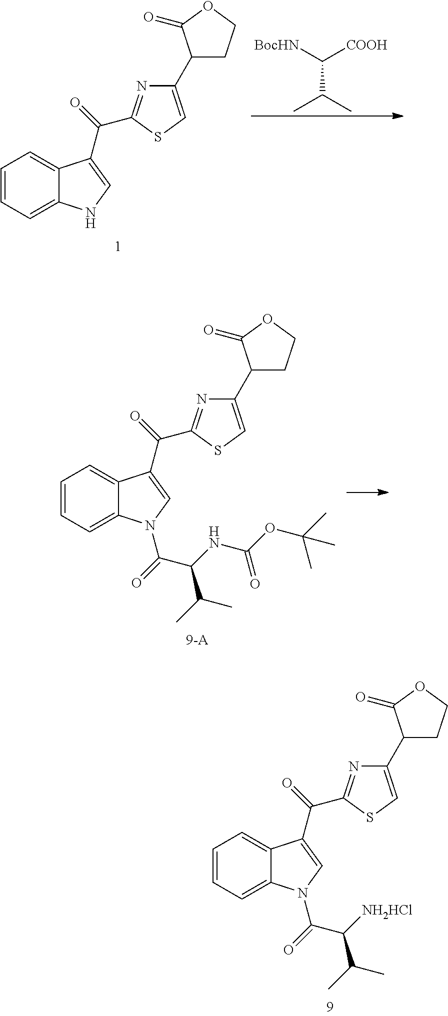

### Step 1: Synthesis of Intermediate 9-A

3-[2-(1H-indole-3-carbonyl)-1,3-thiazol-4-yl]tetrahydrofuran-2-one (0.8 g, 2.56 mmol) was dissolved in N,N-dimethylformamide (15 mL). N-tert-butoxycarbonyl-L-valine (1.7 g, 7.825 mmol), 0-(7-azabenzotriazol-1-yl)-N,N,N,N-tetramethyluronium hexafluorophosphate (2 g, 5.26 mmol), and N,N-diisopropylethylamine (4.7 mL, 27.2 mmol) were added sequentially. The reaction mixture was stirred at room temperature for 18 hours. The reaction was monitored by liquid chromatography-mass spectrometry (LC-MS) until completion. The reaction mixture was diluted with water and extracted with ethyl acetate. The organic phase was dried and concentrated under reduced pressure, followed by purification by silica gel column chromatography to obtain the target product tert-butyl {[(2S)-3-methyl-1-oxo-1-(3-{[4-(2-oxotetrahydrofuran-3-yl)-1,3-thiazol-2-yl]carbonyl}indol-1-yl)butan-2-yl]amino}carboxylate (Intermediate 9-A) (an off-white solid, 1.26 g, 2.46 mmol, a yield of 96.18%).

LCMS (ESI): [M+H]⁺=512.2.

¹H NMR (400 MHz, DMSO-d₆): δ 9.70-9.50 (m, 1H), 8.50-8.34 (m, 2H), 8.18-8.16 (m, 1H), 7.80-7.70 (m, 1H), 7.51-7.48 (m, 2H), 4.82-4.77 (m, 1H), 4.57-4.47 (m, 1H), 4.47-4.34 (m, 2H), 2.82-2.73 (m, 2H), 2.28-2.15 (m, 1H), 1.04-0.98 (m, 3H), 0.97-0.88 (m, 3H).

### Step 2: Synthesis of Compound 9

Tert-butyl {[(2S)-3-methyl-1-oxo-1-(3-{[4-(2-oxotetrahydrofuran-3-yl)-1,3-thiazol-2-yl]carbonyl}indol-1-yl)butan-2-yl]amino}carboxylate (Intermediate 9-A) (0.1 g, 0.195 mmol) was dissolved in ethyl acetate (1.5 mL). A 4 mol/L HCl/ethyl acetate solution (0.5 mL, 2 mmol) was added dropwise under an ice bath. The reaction mixture was stirred at room temperature for 18 hours. The reaction mixture was concentrated under reduced pressure. The residue was treated with n-hexane/tetrahydrofuran to obtain the product 3-[2-({1-[(2S)-2-amino-3-methylbutanoyl]indol-3-yl}carbonyl)-1,3-thiazol-4-yl]tetrahydrofuran-2-one hydrochloride (Compound 9) (a white solid powder, 70 mg, 0.153 mmol, a yield of 78.3%).

LCMS (ESI): [M+H]⁺=412.2.

¹H NMR (400 MHz, DMSO-d₆): δ 9.45-9.35 (m, 1H), 8.88-8.66 (br, 3H), 8.50-8.30 (m, 2H), 8.26-8.14 (m, 1H), 7.62-7.46 (m, 2H), 5.05-4.86 (m, 1H), 4.59-4.49 (m, 1H), 4.49-4.37 (m, 2H), 2.82-2.63 (m, 2H), 2.45-2.32 (m, 1H), 1.13-0.97 (m, 6H).

### Example 10

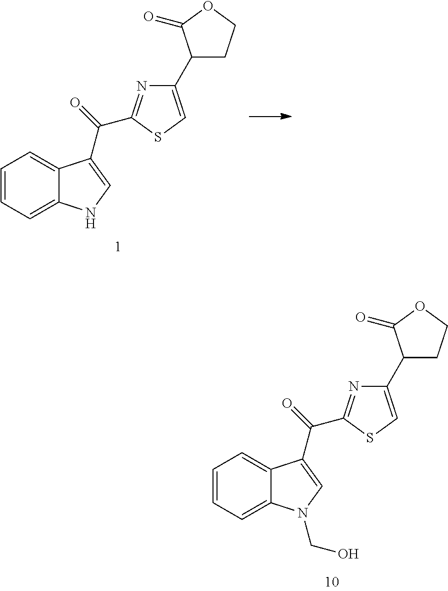 [SMILES: O=C(c1nc(C2CCOC2=O)cs1)c1c[nH]c2ccccc12 ; O=C(c1nc(C2CCOC2=O)cs1)c1cn(CO)c2ccccc12]

### Step 1: Synthesis of Compound 10

3-[2-(1H-indole-3-carbonyl)-1,3-thiazol-4-yl]tetrahydrofuran-2-one (0.5 g, 1.6 mmol) was dissolved in tetrahydrofuran (5 mL). A 37% formaldehyde aqueous solution (5 mL) and tetrabutylammonium fluoride (42 mg, 0.16 mmol) were added. The reaction mixture was stirred at room temperature for 12 hours. The reaction was monitored by LC-MS until completion. The reaction mixture was diluted with ethyl acetate, washed with saturated brine, dried, and concentrated under reduced pressure. The residue was purified by silica gel column chromatography to obtain the product 3-{2-[1-(hydroxymethyl)indole-3-carbonyl]thiazol-4-yl}tetrahydrofuran-2-one (Compound 10) (a white solid, 420 mg, 1.17 mmol, a yield of 73.1%).

LCMS (ESI): [M+H]⁺=343.2.

¹H NMR (400 MHz, DMSO-d₆): δ 9.07 (s, 1H), 8.35-8.33 (m, 1H), 8.05 (s, 1H), 7.74-7.72 (m, 1H), 7.34-7.32 (m, 2H), 6.92-6.88 (t, J=7.6 Hz, 1H), 5.70-5.68 (d, J=7.6 Hz, 2H), 4.60-4.55 (m, 1H), 4.46-4.37 (m, 2H), 2.77-2.72 (m, 2H)

### Example 11

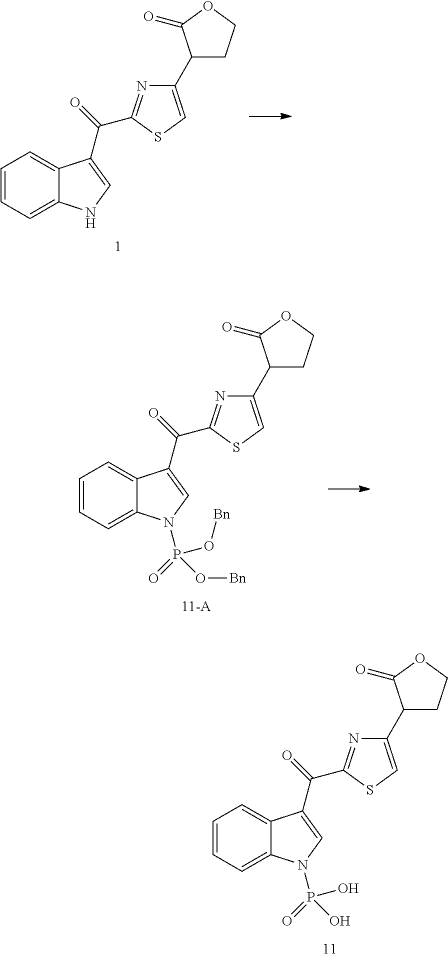 [SMILES: O=C(c1nc(C2CCOC2=O)cs1)c1c[nH]c2ccccc12 ; O=C(c1nc(C2CCOC2=O)cs1)c1cn(P(=O)(OCc2ccccc2)OCc2ccccc2)c2ccccc12 ; O=C(c1nc(C2CCOC2=O)cs1)c1cn(P(=O)(O)O)c2ccccc12]

### Step 1: Synthesis of Intermediate 11-A

3-[2-(1H-indole-3-carbonyl)-1,3-thiazol-4-yl]tetrahydrofuran-2-one (0.5 g, 1.6 mmol) was dissolved in anhydrous tetrahydrofuran (10 mL). A 0.5 mol/L potassium bis(trimethylsilyl)amide toluene solution (3.2 mL) was added dropwise under an ice bath. After stirring for 20 minutes, a 0.4 mol/L dibenzyl phosphorochloridate toluene solution (5 mL) was added dropwise slowly. The reaction mixture was stirred for 2 hours, and the reaction was monitored by LC-MS until completion. Saturated ammonium chloride (2 mL) was added to quench the reaction. The mixture was extracted with ethyl acetate. The organic phase was dried and concentrated under reduced pressure to obtain the product dibenzyl {3-[4-(2-oxotetrahydrofuran-3-yl)thiazole-2-carbonyl]-1H-indol-1-yl}phosphate (crude product 11-A) (a brown solid, 0.92 g, 1.6 mmol).

LCMS (ESI): [M+H]⁺=573.4.

### Step 2: Synthesis of Compound 11

Dibenzyl {3-[4-(2-oxotetrahydrofuran-3-yl)thiazole-2-carbonyl]-1H-indol-1-yl}phosphate (0.92 g, 1.6 mmol) was dissolved in methanol (40 mL). 20% Palladium hydroxide on carbon (1.12 g, 1.6 mmol) was added. After hydrogen displacement, the hydrogenation reaction was carried out for 8 hours. The reaction was monitored by LC-MS until completion. The reaction mixture was filtered and concentrated under reduced pressure. The residue was purified by reverse-phase column chromatography to obtain the product {3-[4-(2-oxotetrahydrofuran-3-yl)thiazole-2-carbonyl]-1H-indol-1-yl}phosphoric acid (Compound 11) (a yellow solid, 0.38 g, 0.97 mmol, a yield of 60.6%).

LCMS (ESI): [M+H]⁺=393.4.

¹H NMR (400 MHz, DMSO-d₆): δ 9.03-9.01 (m, 1H), 8.66 (s, 1H), 8.28-8.20 (m, 1H), 8.01-7.96 (m, 1H), 7.36-7.26 (m, 2H), 4.58-4.52 (m, 1H), 4.50-4.36 (m, 2H), 2.80-2.70 (m, 2H).

### Example 12

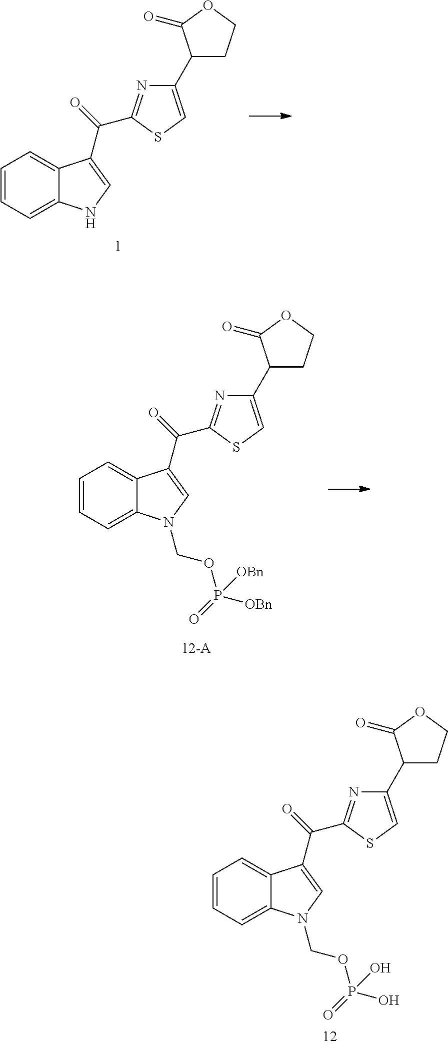 [SMILES: O=C(c1nc(C2CCOC2=O)cs1)c1c[nH]c2ccccc12 ; O=C(c1nc(C2CCOC2=O)cs1)c1cn(COP(=O)(OCc2ccccc2)OCc2ccccc2)c2ccccc12 ; O=C(c1nc(C2CCOC2=O)cs1)c1cn(COP(=O)(O)O)c2ccccc12]

### Step 1: Synthesis of Intermediate 12-A

3-[2-(1H-indole-3-carbonyl)-1,3-thiazol-4-yl]tetrahydrofuran-2-one (0.5 g, 1.6 mmol) was dissolved in anhydrous tetrahydrofuran (10 mL). A 0.5 mol/L potassium bis(trimethylsilyl)amide toluene solution (3.2 mL) was added dropwise under an ice bath. After stirring for 20 minutes, dibenzyl chloromethylphosphonate (522 mg, 1.6 mmol) was added. The reaction mixture was stirred at room temperature overnight. The reaction was monitored by LC-MS until completion. The reaction mixture was concentrated under reduced pressure. The residue was purified by silica gel column chromatography to obtain the product dibenzyl {3-[4-(2-oxotetrahydrofuran-3-yl)thiazole-2-carbonyl]-1H-indol-1-yl}methylphosphate (Intermediate 12-A) (a yellow oil, 512 mg, 0.85 mmol).

LCMS (ESI): [M+H]⁺=603.4.

### Step 2: Synthesis of Compound 12

Dibenzyl {3-[4-(2-oxotetrahydrofuran-3-yl)thiazole-2-carbonyl]-1H-indol-1-yl}methylphosphate (0.51 g, 0.85 mmol) was dissolved in methanol (20 mL). 20% Palladium hydroxide on carbon (0.6 g, 0.85 mmol) was added. After hydrogen displacement, the hydrogenation reaction was carried out for 8 hours. The reaction was monitored by LC-MS until completion. The reaction mixture was filtered and concentrated under reduced pressure. The residue was purified by reverse-phase column chromatography to obtain the product {3-[4-(2-oxotetrahydrofuran-3-yl)thiazole-2-carbonyl]-1H-indol-1-yl}methylphosphoric acid (Compound 12) (a yellow solid, 64 mg, 0.15 mmol, a yield of 17.6%).

LCMS (ESI): [M+H]⁺=423.4.

¹H NMR (400 MHz, DMSO-d₆): δ 9.05-9.01 (m, 1H), 8.67-8.62 (m, 1H), 8.30-8.25 (m, 1H), 7.81-7.78 (m, 1H), 7.46-7.39 (m, 2H), 5.90-5.85 (m, 2H), 4.60-4.55 (m, 1H), 4.50-4.37 (m, 2H), 2.84-2.72 (m, 2H).

### Example 13

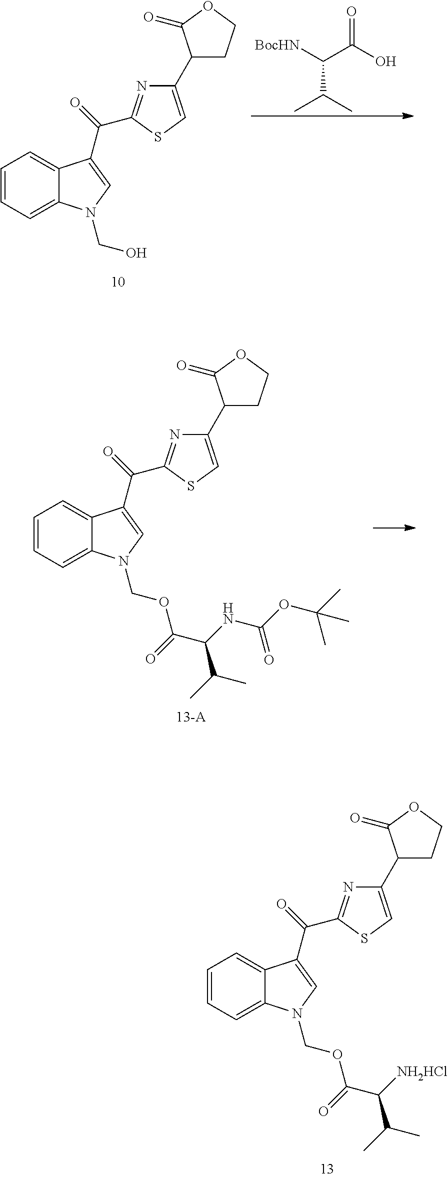

### Step 1: Synthesis of Intermediate 13-A

3-{2-[1-(hydroxymethyl)indole-3-carbonyl]thiazol-4-yl}tetrahydrofuran-2-one (Compound 10) (0.44 g, 1.28 mmol) was dissolved in N,N-dimethylformamide (6 mL). N-tert-butoxycarbonyl-L-valine (0.42 g, 1.92 mmol), 0-(7-azabenzotriazol-1-yl)-N,N,N,N-tetramethyluronium hexafluorophosphate (0.73 g, 1.92 mmol), and N,N-diisopropylethylamine (0.67 mL, 3.84 mmol) were added sequentially. After stirring at room temperature for 18 hours, the reaction was monitored by LC-MS until completion. The reaction mixture was diluted with ethyl acetate and washed with saturated brine. The organic phase was dried and concentrated under reduced pressure. The residue was purified by silica gel column chromatography to obtain the product {3-[4-(2-oxotetrahydrofuran-3-yl)thiazole-2-carbonyl]-1H-indol-1-yl}-N-tert-butoxycarbonyl-L-valine methyl ester (13-A) (a colorless oil, 206 mg, 0.38 mmol, a yield of 29.7%).

LCMS (ESI): [M+H]⁺=542.2.

### Step 2: Synthesis of Compound 13

{3-[4-(2-oxotetrahydrofuran-3-yl)thiazole-2-carbonyl]-1H-indol-1-yl}-N-tert-butoxycarbonyl-L-valine methyl ester (13-A) (0.2 g, 0.37 mmol) was dissolved in ethyl acetate (3 mL). A 4 mol/L HCl/ethyl acetate solution (1 mL, 4 mmol) was added under an ice bath. The reaction mixture was stirred at room temperature for 18 hours. The reaction system was concentrated under reduced pressure. The residue was treated with n-hexane/tetrahydrofuran to obtain the product {3-[4-(2-oxotetrahydrofuran-3-yl)thiazole-2-carbonyl]-1H-indol-1-yl}-L-valine methyl ester hydrochloride (Compound 13) (a white solid powder, 72 mg, 0.15 mmol, a yield of 40.8%).

LCMS (ESI): [M+H]⁺=442.2.

¹H NMR (400 MHz, DMSO-d₆): δ 9.20 (s, 1H), 8.82-8.68 (br, 3H), 8.40-8.38 (m, 1H), 8.13 (s, 1H), 7.81-7.76 (m, 1H), 7.45-7.40 (m, 2H), 6.60-6.42 (m, 2H), 5.02-4.96 (m, 1H), 4.74-4.64 (m, 1H), 4.53-4.42 (m, 2H), 2.85-2.82 (m, 2H), 2.12-1.98 (m, 1H), 1.13-0.97 (m, 6H).

### Example 14

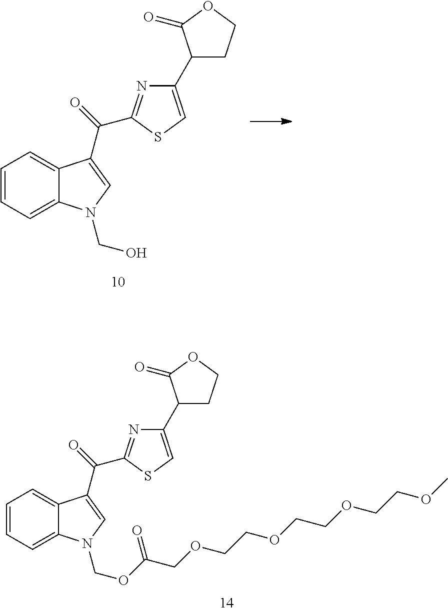 [SMILES: O=C(c1nc(C2CCOC2=O)cs1)c1cn(CO)c2ccccc12 ; COCCOCCOCCOCC(=O)OCn1cc(C(=O)c2nc(C3CCOC3=O)cs2)c2ccccc21]

### Step 1: Synthesis of Compound 14

3-{2-[1-(hydroxymethyl)indole-3-carbonyl]thiazol-4-yl}tetrahydrofuran-2-one (Compound 10) (0.2 g, 0.58 mmol) was dissolved in N,N-dimethylformamide (5 mL). 3,6,9,12-tetraoxatridecanoic acid (129 mg, 0.58 mmol), 0-(7-azabenzotriazol-1-yl)-N,N,N,N-tetramethyluronium hexafluorophosphate (0.33 g, 0.87 mmol), and N,N-diisopropylethylamine (0.2 mL, 1.16 mmol) were added sequentially. The reaction mixture was stirred at room temperature for 18 hours, and the reaction was monitored by LC-MS until completion. The reaction system was diluted with water and extracted with ethyl acetate. The organic phase was dried and concentrated under reduced pressure. The residue was purified by silica gel column chromatography to obtain the product 3,6,9,12-tetraoxatridecanoic acid {3-[4-(2-oxotetrahydrofuran-3-yl)thiazole-2-carbonyl]-1H-indol-1-yl}methyl ester (Compound 14) (a colorless oil, 0.115 g, 0.21 mmol, a yield of 36.2%).

LCMS (ESI): [M+H]⁺=547.2.

¹H NMR (400 MHz, DMSO-d₆): δ 9.10 (s, 1H), 8.45-8.40 (m, 1H), 8.32 (s, 1H), 7.70-7.60 (m, 1H), 7.40-7.35 (m, 2H), 6.28 (s, 2H), 4.55-4.50 (m, 1H), 4.48-4.38 (m, 2H), 4.20 (s, 2H), 3.72-3.65 (m, 4H), 3.64-3.58 (m, 6H), 3.54-3.52 (m, 2H), 3.36 (s, 3H), 2.78-2.70 (m, 2H).

### Example 15

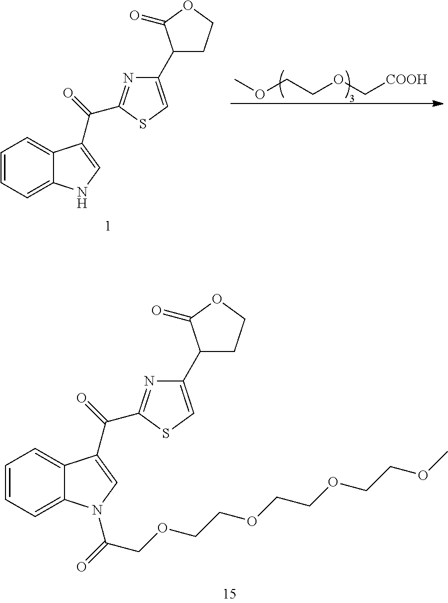

### Step 1: Synthesis of Compound 15

3-[2-(1H-indole-3-carbonyl)-1,3-thiazol-4-yl]tetrahydrofuran-2-one (0.4 g, 1.28 mmol) was dissolved in N,N-dimethylformamide (6 mL). 3,6,9,12-tetraoxatridecanoic acid (568 mg, 2.56 mmol), 0-(7-azabenzotriazol-1-yl)-N,N,N,N-tetramethyluronium hexafluorophosphate (0.97 g, 2.56 mmol), and N,N-diisopropylethylamine (1.34 mL, 7.68 mmol) were added sequentially. The reaction mixture was stirred at room temperature for 18 hours, and the reaction was monitored by LC-MS until completion. The reaction system was diluted with water and extracted with ethyl acetate. The organic phase was dried and concentrated under reduced pressure. The residue was purified by silica gel column chromatography to obtain the product 3-{2-[1-(3,6,9,12-tetraoxatridecanoyl)-1H-indole-3-carbonyl]thiazol-4-yl}dihydrofuran-2(3H)-one (Compound 15) (a colorless oil, 0.21 g, 0.4 mmol, a yield of 31.5%).

LCMS (ESI): [M+H]⁺=517.2.

¹H NMR (400 MHz, DMSO-d₆): δ 9.11 (s, 1H), 8.48-8.42 (m, 1H), 8.33 (s, 1H), 7.72-7.66 (m, 1H), 7.42-7.36 (m, 2H), 4.58-4.52 (m, 1H), 4.50-4.41 (m, 2H), 4.22 (s, 2H), 3.74-3.65 (m, 4H), 3.64-3.56 (m, 6H), 3.54-3.52 (m, 2H), 3.38 (s, 3H), 2.77-2.72 (m, 2H).

### Efficacy Example 1: Human AHR Agonist Luciferase Assay

Cytochrome P450 family 1 member A1 (CYP1A1) is one of the hallmark genes regulated by the AHR signaling pathway. The upstream promoter of the CYP1A1 gene includes a specific sequence called a dioxin responsive element (DRE), which binds to an activated AHR to initiate CYP1A1 expression. To detect AHR activation, a reporter gene plasmid was constructed by placing the gene sequence from −1200 bp to 0 bp upstream of CYP1A1 in front of a Nano-luc luciferase reporter gene according to conventional construction methods of reporter gene plasmids in the art. The reporter gene plasmid was transfected into human hepatoma HepG2 cells by a conventional liposome transfection method in the art, and stably expressing cells were selected by puromycin. The selected cells were diluted to a concentration of 5 cells/mL and seeded into a 96-well plate at 100 μL/well, and single clones were obtained after growth. A single clone with normal morphology, normal growth, and the highest induction signal was selected for the human AHR agonist luciferase assay.

On day one, the HepG2 monoclonal cell line stably expressing reporter gene was counted and seeded into a white opaque 96-well plate at a density of 1×10⁴ cells per well −8×10⁴ cells per well using 100 μL of complete medium without tryptophan and phenol red (minimal essential medium (MEM) without tryptophan and phenol red, 10% (v/v) fetal bovine serum, 1× non-essential amino acids, 1× sodium pyruvate, and 1× GlutaMax). The cells were cultured in an incubator maintained at 37° C. with 5% (v/v) CO₂ for 24 hours.

Test compounds were prepared as 10 mmol/L stock solutions in dimethyl sulfoxide (DMSO) and stored at 4° C. protected from light. Before treatment, the compound stock solutions, positive control compounds (e.g., dioxin, 2-(1H-indole-3-carbonyl)thiazole-4-carboxylic acid methyl ester (ITE), or kynurenine), and a negative control compound (DMSO) were subjected to preliminary dilution (determined based on pre-experiments) with the complete medium without tryptophan and phenol red, followed by 3- to 20-fold serial dilutions to 11 concentration points to obtain 2× dilution solutions. When treating the cells, 50 μL of medium was first aspirated from the 96-well plate containing the seeded cells, and then 50 μL of the serially diluted 2× dilution solution was added. Each compound was tested in duplicate, and each duplicate experiment included a well with a concentration of 0 (i.e., containing only the medium) to determine the baseline. The reporter cells were returned to the incubator and incubated for 4 to 24 hours after treatment.

After treatment, 100 μL/well of Nano-Glo Luciferase Assay Reagent was added to the 96-well plate, and the relative light unit (RLU) of each well was measured. The RLU values for the wells with the concentration of 0 on the 96-well plates were averaged to calculate the baseline Ave RLU^{Vehicle}. The activity of AHR induced by the test compounds at different concentrations was calculated according to Formula (1) based on the ratio of RLU^{Test Cmpd} from the experimental groups to the baseline Ave RLU^{Vehicle}, to determine the activation fold.

$$activation fold = \frac{{RLU}^{Test Cmpd}}{Ave {RELU}^{Vehicle}} . \qquad Formula ( 1 )$$

The activation fold and the corresponding compound concentration were fitted using GraphPad Prism 9 with [Agonist] vs. response—variable slope (four parameters) to calculate the EC₅₀ of the compound for AHR activation. The EC₅₀ values for the compounds are shown in Table 8, where A indicates EC₅₀≤500 nM, B indicates 500 nM<EC₅₀≤2.0 μM, and C indicates 2.0 μM<EC₅₀≤100 μM.

### Efficacy Example 2: Mouse AHR Agonist Luciferase Assay

To detect AHR activation, a reporter gene plasmid was constructed by placing six tandem xenobiotic responsive elements (XREs, see Buckley, S. M. K. et al., Sci. Rep. 2015; 5: 11842.) in front of a Nano-luc luciferase reporter gene according to conventional construction methods of reporter gene plasmids in the art. The reporter gene plasmid was transfected into mouse hepatoma Hepa1-6 cells by a conventional liposome transfection method in the art, and stably expressing cells were selected by puromycin for the mouse AHR agonist luciferase assay.

On day one, the stably expressing reporter gene Hepa1-6 cell line was counted and seeded into a white opaque 96-well plate at a density of 1×10⁴ cells per well −8×10⁴ cells per well using 100 μL of phenol red-free Dulbecco's Modified Eagle Medium (DMEM) (DMEM without phenol red, 10% fetal bovine serum). The cells were cultured in an incubator maintained at 37° C. with 5% CO₂ for 24 hours.

Test compounds were prepared as 10 mM stock solutions in DMSO and stored at 4° C. protected from light. Before treatment, the compound stock solutions, positive control compounds (e.g., dioxin, ITE, or kynurenine), and a negative control compound (DMSO) were subjected to preliminary dilution (determined based on pre-experiments) with complete medium without tryptophan and phenol red, followed by 3- to 20-fold serial dilutions to 11 concentration points to obtain 10× dilution solutions. When treating the cells, 10 μL of medium was first aspirated from the 96-well plate containing the seeded cells, and then 10 μL of the serially diluted 10× dilution solution was added. Each compound was tested in duplicate, and each duplicate experiment included a well with a concentration of 0 (i.e., containing only medium) to determine the baseline. The reporter cells were returned to the incubator and incubated for 4 to 24 hours after treatment.

After treatment, 100 μL/well of Nano-Glo Luciferase Assay Reagent was added to the 96-well plate, and the RLU of each well was measured. The RLU values for the wells with the concentration of 0 on the 96-well plates were averaged to calculate the baseline Ave RLU^{Vehicle} The activity of AHR induced by the test compounds at different concentrations was calculated according to Formula (1) based on the ratio of RLU^{Test Cmpd} from the experimental groups to the baseline Ave RLU^{Vehicle}, to determine the activation fold.

$$activation fold = \frac{{RLU}^{Test Cmpd}}{Ave {RELU}^{Vehicle}} . \qquad Formula ( 1 )$$

The activation fold and the corresponding compound concentration were fitted using GraphPad Prism 9 with [Agonist] vs. response—variable slope (four parameters) to determine the EC₅₀ of the compound for AHR activation. The EC₅₀ values for the compounds are shown in Table 8, where A indicates EC₅₀≤100 nM, B indicates 100 nM<EC₅₀≤2.0 μM, and C indicates 2.0 μM<EC₅₀≤100 μM.

### Efficacy Example 3: Rat AHR Agonist Luciferase Assay

To detect AHR activation, a Nano-luc luciferase reporter gene linked to six tandem xenobiotic responsive elements (XREs) (see Buckley, S. M. K. et al., Sci. Rep. 2015; 5: 11842.) was synthesized and cloned into a lentiviral transfer plasmid pGWLV11-new using KpnI and XbaI restriction sites. A lentivirus containing the reporter gene was generated by co-transfecting the transfer plasmid with packaging plasmids into 293T cells using conventional transfection methods in the art, followed by purification. The reporter gene was introduced into a genome of rat hepatoma H-4-II-E cells via lentiviral infection, and cells stably expressing the reporter gene were selected with puromycin for the rat AHR agonist luciferase assay.

On day one, the H-4-II-E cell line stably expressing the reporter gene was counted and seeded into a white opaque 96-well plate at a density of 1×10⁴ cells per well −8×10⁴ cells per well using 100 μL of complete medium without tryptophan and phenol red (MEM without tryptophan and phenol red, 10% fetal bovine serum, 1× non-essential amino acids, 1× sodium pyruvate, and 1× GlutaMax). The cells were cultured in an incubator maintained at 37° C. with 5% CO₂ for 24 hours.

Test compounds were prepared as 10 mM stock solutions in DMSO and stored at 4° C. protected from light. Before treatment, the compound stock solutions, positive control compounds (e.g., dioxin, ITE, or kynurenine), and a negative control compound (DMSO) were subjected to a preliminary dilution (determined based on pre-experiments) with the complete medium without tryptophan and phenol red, followed by 3- to 20-fold serial dilutions to 11 concentration points to obtain 2× dilution solutions. When treating the cells, 50 μL of medium was first aspirated from the 96-well plate containing the seeded cells, and then 50 μL of the serially diluted 2× dilution solutions was added. Each compound was tested in duplicate, and each duplicate experiment included a well with a concentration of 0 (i.e., containing only the medium) to determine the baseline. The reporter cells were returned to the incubator and incubated for 4 to 24 hours after treatment.

After treatment, 100 μL/well of Nano-Glo Luciferase Assay Reagent was added to the 96-well plates, and the RLU of each well was measured. The RLU values for the wells with the concentration of 0 on the 96-well plates were averaged to calculate the baseline Ave RLU^{Vehicle}. The activity of AHR induced by the test compounds at different concentrations was calculated according to Formula (1) based on the ratio of RLU^{Test Cmpd} from the experimental groups to the baseline Ave RLU^{Vehicle}, to determine the activation fold.

$$activation fold = \frac{{RLU}^{Test Cmpd}}{Ave {RELU}^{Vehicle}} . \qquad Formula ( 1 )$$

The activation fold and the corresponding compound concentration were fitted using GraphPad Prism 9 with [Agonist] vs. response—variable slope (four parameters) to calculate the EC₅₀ of the compound for AHR activation. The EC₅₀ values for the compounds are shown in Table 8, where A indicates EC₅₀≤10 nM, B indicates 10 nM<EC₅₀≤2.0 μM, and C indicates 2.0 μM<EC₅₀≤100 μM.

TABLE 8
EC₅₀ VALUES OF THE COMPOUNDS

| | HepG2 | Hepa 1-6 | H4IIE |
|---|---|---|---|
| Compound No. | EC₅₀ | EC₅₀ | EC₅₀ |
| 1 | A | A | A |
| 1-isomer-A | A | A | A |
| 1-isomer-B | A | A | A |
| 1-2 | A | A | A |
| 1-3 | A | A | A |
| 1-4 | A | A | A |
| 1-5 | A | A | A |
| 1-6 | A | A | A |
| 1-7 | A | — | — |
| 1-8 | A | — | — |
| 1-9 | A | — | — |
| 2 | A | A | A |
| 2-2 | A | A | A |
| 2-3 | A | A | A |
| 2-4 | A | A | — |
| 2-5 | A | A | — |
| 3 | A | A | — |
| 3-2 | A | A | — |
| 3-3 | A | A | — |
| 3-4 | A | B | — |
| 3-5 | B | — | — |
| 4 | A | A | A |
| 4-2 | A | A | A |
| 4-3 | A | — | — |
| 4-4 | A | — | — |
| 4-5 | A | — | — |
| 5 | A | A | — |
| 6 | B | — | — |
| 7 | A | — | C |
| 7-2 | A | — | C |
| 7-3 | A | — | C |
| 7-4 | A | — | C |
| 7-5 | A | A | C |
| 7-6 | A | A | C |
| 8 | A | A | — |
| 8-cis-1 | A | — | — |
| 8-cis-2 | A | — | — |
| 8-trans-1 | A | — | — |
| 8-trans-2 | A | — | — |
| 8-1 | A | A | — |
| 8-2 | A | A | — |
| 8-3 | A | A | — |
| 8-4 | A | A | — |
| 8-5 | A | A | — |
| 8-6 | A | A | — |
| 9 | B | B | — |
| 10 | B | B | — |
| 11 | B | B | — |
| 12 | B | B | — |
| 13 | B | B | — |
| 14 | B | B | — |
| 15 | B | B | — |
| ITE | A | B | B |

As shown in Table 8, the compounds of the present disclosure activate expression of CYP1A1 gene and exhibit a strong agonistic activity on AHR.

What is claimed is:

1. An aryl hydrocarbon receptor modulator, or an enantiomer, a prodrug, or a pharmaceutically acceptable salt thereof, wherein the aryl hydrocarbon receptor modulator comprises the following general formula (I): 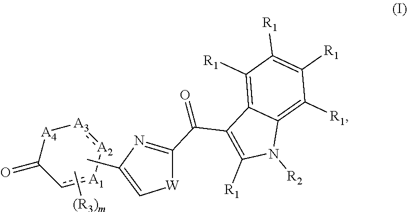
wherein
W is C═C or S;
A₁, A₂, and A₃ are each independently a bond or C; m is an integer from 1 to 7; R₃ is independently H, D, halogen, CN, OR, SR, N(R)R, —C(O)R, —C(O)OR, RC(O)O—, S(O)ₙR, unsubstituted C1-C8 alkyl or substituted C1-C8 alkyl with 1-6 Rₐ, unsubstituted C2-C8 alkenyl or substituted C2-C8 alkenyl with 1-6 Rₐ, unsubstituted C2-C8 alkynyl or substituted C2-C8 alkynyl with 1-6 Rₐ, or unsubstituted C3-C10 cycloalkyl or substituted C3-C10 cycloalkyl with 1-6 Rₐ, wherein Rₐ is D, halogen, OR, SR, N(R)R, —C(O)R, —C(O)OR, RC(O)O—, or S(O)ₙR;
A₄ is independently O or NR₄, wherein R₄ is H, unsubstituted C1-C8 alkyl or substituted C1-C8 alkyl with 1-6 R_{b}, unsubstituted C2-C8 alkenyl or substituted C2-C8 alkenyl with 1-6 R_{b}, unsubstituted C2-C8 alkynyl or substituted C2-C8 alkynyl with 1-6 R_{b}, or unsubstituted C3-C10 cycloalkyl or substituted C3-C10 cycloalkyl with 1-6 R_{b}; and R_{b} is D, halogen, OR, SR, N(R)R, —C(O)R, —C(O)OR, RC(O)O—, or S(O)ₙR;
R₁ is independently H, D, halogen, CN, OR, SR, N(R)R, —C(O)R, —C(O)OR, RC(O)O—, S(O)ₙR, unsubstituted C1-C8 alkyl or substituted C1-C8 alkyl with 1-6 R_{b}, unsubstituted C2-C8 alkenyl or substituted C2-C8 alkenyl with 1-6 R_{b}, unsubstituted C2-C8 alkynyl or substituted C2-C8 alkynyl with 1-6 R_{b}, or unsubstituted C3-C10 cycloalkyl or substituted C3-C10 cycloalkyl with 1-6 R_{b}; or two adjacent or meta R₁ groups form a 3-10 membered carbocycle or a 3-10 membered heterocycle containing 1 to 3 heteroatoms selected from N, O, or S, wherein
n is 1 or 2;
R is independently H, C1-C8 alkyl, C2-C8 alkenyl, C2-C8 alkynyl, or C3-C10 cycloalkyl; and
R₂ is H, —(CH₂O)ₐC(O)(CH₂)_{b}(O)_{c}(CH(R_{c}))_{d}(CH₂CH₂O)ₑR_{c}, —(CH₂O)ₐP(O)(Rₚ)₂, or —(CH₂O)ₐ(CH(R_{c}))_{d}(CH₂CH₂O)ₑR_{c}, wherein Rₚ is independently selected form (CH₂)_{b}(O)_{c}(CH(R_{c}))_{d}(CH₂CH₂O)ₑR_{c}; R_{c} is independently H, D, C1-C8 alkyl, NH₂—, (C1-C8 alkyl)NH—, (C1-C8 alkyl)₂N—, NH₂(C1-C8 alkyl)-, OH, HO—(C1-C8 alkyl)-, or (C1-C8 alkyl)O—; a, b, c, and d are each independently 0 or 1; and e is an integer from 0 to 3.

2. The aryl hydrocarbon receptor modulator according to claim 1, wherein when W is S, the general formula (I) is a general formula (Ia):

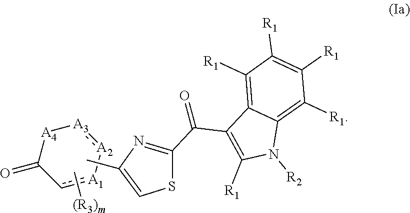

3. The aryl hydrocarbon receptor modulator according to claim 2, wherein
when A₄ is O, the general formula (Ia) is a general formula (Ia1): 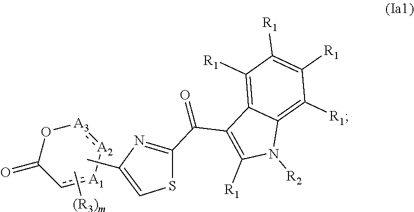
when A₄ is NR₄, the general formula (Ia) is a general formula (Ia2):

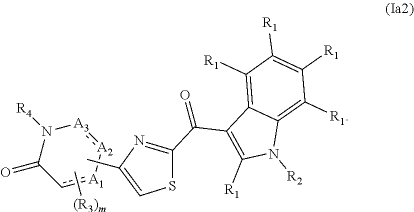

4. The aryl hydrocarbon receptor modulator according to claim 1, wherein when W is C═C, the general formula (I) is a general formula (Ib):

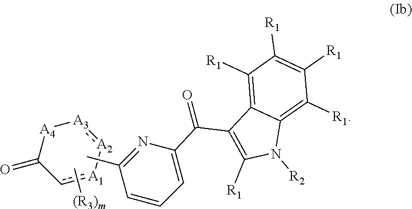

5. The aryl hydrocarbon receptor modulator according to claim 4, wherein
when A₄ is 0, the general formula (Ib) is a general formula (Ib1): 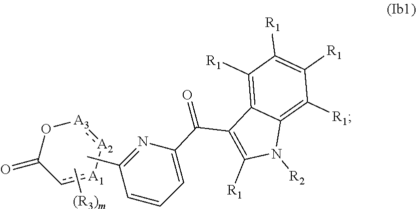
when A₄ is NR₄, the general formula (Ib) is a general formula (Ib2):

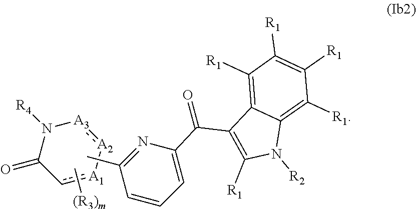

6. The aryl hydrocarbon receptor modulator according to claim 1, wherein
A₁, A₂, and A₃ are each independently a bond or C; m is an integer from 1 to 5; R₃ is independently H, D, halogen, CN, OR, SR, N(R)R, —C(O)R, —C(O)OR, RC(O)O—, S(O)ₙR, unsubstituted C1-C6 alkyl or substituted C1-C6 alkyl with 1-3 Rₐ, unsubstituted C2-C6 alkenyl or substituted C2-C6 alkenyl with 1-3 Rₐ, unsubstituted C2-C6 alkynyl or substituted C2-C6 alkynyl with 1-3 Rₐ, or unsubstituted C3-C8 cycloalkyl or substituted C3-C8 cycloalkyl with 1-3 Rₐ; and Rₐ is D, halogen, OR, SR, N(R)R, —C(O)R, —C(O)OR, RC(O)O—, or S(O)ₙR;
n is 1 or 2; and
R is independently H, C1-C6 alkyl, C2-C6 alkenyl, C2-C6 alkynyl, or C3-C8 cycloalkyl;
preferably,
A₁, A₂, and A₃ are each independently a bond or C; m is an integer from 1 to 3; R₃ is independently H, D, halogen, CN, OR, SR, N(R)R, —C(O)R, —C(O)OR, RC(O)O—, S(O)ₙR, unsubstituted C1-C4 alkyl or substituted C1-C4 alkyl with 1-2 Rₐ, unsubstituted C2-C4 alkenyl or substituted C2-C4 alkenyl with 1-2 Rₐ, unsubstituted C2-C4 alkynyl or substituted C2-C4 alkynyl with 1-2 Rₐ, or unsubstituted C3-C7 cycloalkyl or substituted C3-C7 cycloalkyl with 1-2 Rₐ; and Rₐ is D, halogen, OR, SR, N(R)R, —C(O)R, —C(O)OR, RC(O)O—, or S(O)ₙR;
n is 1 or 2; and
R is independently H, C1-C4 alkyl, C2-C4 alkenyl, C2-C4 alkynyl, or C3-C7 cycloalkyl;
more preferably,
A₁, A₂, and A₃ are each independently a bond or C; m is an integer from 1 to 2; R₃ is independently H, D, halogen, CN, OR, SR, N(R)R, —C(O)R, —C(O)OR, RC(O)O—, S(O)ₙR, unsubstituted C1-C3 alkyl or substituted C1-C3 alkyl with 1 Rₐ, unsubstituted C2-C3 alkenyl or substituted C2-C3 alkenyl with 1 Rₐ, unsubstituted C2-C3 alkynyl or substituted C2-C3 alkynyl with 1 Rₐ, or unsubstituted C5-C6 cycloalkyl or substituted C5-C6 cycloalkyl with 1 Rₐ; and Rₐ is D, halogen, OR, SR, N(R)R, —C(O)R, —C(O)OR, RC(O)O—, or S(O)ₙR;
n is 1 or 2; and
R is independently H, C1-C3 alkyl, C2-C3 alkenyl, C2-C3 alkynyl, or C5-C6 cycloalkyl.

7. The aryl hydrocarbon receptor modulator according to claim 1, wherein
R₁ is independently H, D, halogen, CN, OR, SR, N(R)R, —C(O)R, —C(O)OR, RC(O)O—, S(O)ₙR, unsubstituted C1-C6 alkyl or substituted C1-C6 alkyl with 1-3 R_{b}, unsubstituted C2-C6 alkenyl or substituted C2-C6 alkenyl with 1-3 R_{b}, unsubstituted C2-C6 alkynyl or substituted C2-C6 alkynyl with 1-3 R_{b}, or unsubstituted C3-C8 cycloalkyl or substituted C3-C8 cycloalkyl with 1-3 R_{b}; R_{b} is D, halogen, OR, SR, N(R)R, —C(O)R, —C(O)OR, RC(O)O—, or S(O)ₙR; or two adjacent or meta R₁ groups form a 3-8 membered carbocycle or a 3-8 membered heterocycle containing 1-3 heteroatoms selected from N, O, or S;
n is 1 or 2; and
R is independently H, C1-C6 alkyl, C2-C6 alkenyl, C2-C6 alkynyl, or C3-C8 cycloalkyl;
preferably,
R₁ is independently H, D, halogen, CN, OR, SR, N(R)R, —C(O)R, —C(O)OR, RC(O)O—, S(O)ₙR, unsubstituted C1-C4 alkyl or substituted C1-C4 alkyl with 1-2 R_{b}, unsubstituted C2-C4 alkenyl or substituted C2-C4 alkenyl with 1-2 R_{b}, unsubstituted C2-C4 alkynyl or substituted C2-C4 alkynyl with 1-2 R_{b}, or unsubstituted C3-C7 cycloalkyl or substituted C3-C7 cycloalkyl with 1-2 R_{b}; R_{b} is D, halogen, OR, SR, N(R)R, —C(O)R, —C(O)OR, RC(O)O—, or S(O)ₙR; or two adjacent or meta R₁ groups form a 3-7 membered carbocycle or a 3-7 membered heterocycle containing 1-2 heteroatoms selected from N, O, or S;
n is 1 or 2; and
R is independently H, C1-C4 alkyl, C2-C4 alkenyl, C2-C4 alkynyl, or C3-C7 cycloalkyl;
more preferably,
R₁ is independently H, D, halogen, CN, OR, SR, N(R)R, —C(O)R, —C(O)OR, RC(O)O—, S(O)ₙR, unsubstituted C1-C3 alkyl or substituted C1-C3 alkyl with 1 R_{b}, unsubstituted C2-C3 alkenyl or substituted C2-C3 alkenyl with 1 R_{b}, unsubstituted C2-C3 alkynyl or substituted C2-C3 alkynyl with 1 R_{b}, or unsubstituted C5-C6 cycloalkyl or substituted C5-C6 cycloalkyl with 1 R_{b}; R_{b} is D, halogen, OR, SR, N(R)R, —C(O)R, —C(O)OR, RC(O)O—, or S(O)ₙR; or two adjacent or meta R₁ groups form a 5-6 membered carbocycle or a 5-6 membered heterocycle containing 1-2 heteroatoms selected from N, O, or S;
n is 1 or 2; and
R is independently H, C1-C3 alkyl, C2-C3 alkenyl, C2-C3 alkynyl, or C5-C6 cycloalkyl.

8. The aryl hydrocarbon receptor modulator according to claim 1, wherein
R₂ is H, —(CH₂O)ₐC(O)(CH₂)_{b}(O)_{c}(CH(R_{c}))_{d}(CH₂CH₂O)ₑR_{c}, —(CH₂O)ₐP(O)(Rₚ)₂, or —(CH₂O)ₐ(CH(R_{c}))_{d}(CH₂CH₂O)ₑR_{c}, Rₚ is independently selected from (CH₂)_{b}(O)_{c}(CH(R_{c}))_{d}(CH₂CH₂O)ₑR_{c}; R_{c} is independently H, D, C1-C6 alkyl, NH₂—, (C1-C6 alkyl)NH—, (C1-C6 alkyl)₂N—, NH₂(C1-C6 alkyl)-, OH, HO—(C1-C6 alkyl)-, or (C1-C6 alkyl)O—; a, b, c, and d are each independently 0 or 1; and e is an integer from 0 to 2;
preferably,
R₂ is H, —(CH₂O)ₐC(O)(CH₂)_{b}(O)_{c}(CH(R_{c}))_{d}(CH₂CH₂O)ₑR_{c}, —(CH₂O)ₐP(O)(Rₚ)₂, or —(CH₂O)ₐ(CH(R_{c}))_{d}(CH₂CH₂O)ₑR_{c}; Rₚ is independently selected from (CH₂)_{b}(O)_{c}(CH(R_{c}))_{d}(CH₂CH₂O)ₑR_{c}; R_{c} is independently H, D, C1-C4 alkyl, NH₂—, (C1-C4 alkyl)NH—, (C1-C4 alkyl)₂N—, NH₂(C1-C4)alkyl-, OH, HO—(C1-C4)alkyl-, or (C1-C4 alkyl)O—; a, b, c, and d are each independently 0 or 1; and e is an integer of 0 or 1;
more preferably,
R₂ is H, —(CH₂O)ₐC(O)(CH₂)_{b}(O)_{c}(CH(R_{c}))_{d}(CH₂CH₂O)ₑR_{c}, —(CH₂O)ₐP(O)(Rₚ)₂, or —(CH₂O)ₐ(CH(R_{c}))_{d}(CH₂CH₂O)ₑR_{c}; Rₚ is independently selected from (CH₂)_{b}(O)_{c}(CH(R_{c}))_{d}(CH₂CH₂O)ₑR_{c}; R_{c} is independently H, D, C1-C3 alkyl, NH₂—, (C1-C3 alkyl)NH—, (C1-C3 alkyl)₂N—, NH₂(C1-C3)alkyl-, OH, HO—(C1-C3)alkyl-, or (C1-C3 alkyl)O—; a, b, c, and d are each independently 0 or 1; and e is an integer of 0 or 1.

9. The aryl hydrocarbon receptor modulator according to claim 1, wherein
R₄ is H, unsubstituted C1-C6 alkyl or substituted C1-C6 alkyl with 1-3 R_{b}, unsubstituted C2-C6 alkenyl or substituted C2-C6 alkenyl with 1-3 R_{b}, unsubstituted C2-C6 alkynyl or substituted C2-C6 alkynyl with 1-3 R_{b}, or unsubstituted C3-C8 cycloalkyl or substituted C3-C8 cycloalkyl with 1-3 R_{b}; and R_{b} is D, halogen, OR, SR, N(R)R, —C(O)R, —C(O)OR, RC(O)O—, or S(O)ₙR; and
R is independently H, C1-C6 alkyl, C2-C6 alkenyl, C2-C6 alkynyl, or C3-C8 cycloalkyl;
preferably,
R₄ is H, unsubstituted C1-C4 alkyl or substituted C1-C4 alkyl with 1-2 R_{b}, unsubstituted C2-C4 alkenyl or substituted C2-C4 alkenyl with 1-2 R_{b}, unsubstituted C2-C4 alkynyl or substituted C2-C4 alkynyl with 1-2 R_{b}, or unsubstituted C3-C7 cycloalkyl or substituted C3-C7 cycloalkyl with 1-2 R_{b}; and R_{b} is D, halogen, OR, SR, N(R)R, —C(O)R, —C(O)OR, RC(O)O—, or S(O)ₙR; and
R is independently H, C1-C4 alkyl, C2-C4 alkenyl, C2-C4 alkynyl, or C3-C7 cycloalkyl;
more preferably,
R₄ is H, unsubstituted C1-C3 alkyl or substituted C1-C3 alkyl with 1 R_{b}, unsubstituted C2-C3 alkenyl or substituted C2-C3 alkenyl with 1 R_{b}, unsubstituted C2-C3 alkynyl or substituted C2-C3 alkynyl with 1 R_{b}, or unsubstituted C5-C6 cycloalkyl or substituted C5-C6 cycloalkyl with 1 R_{b}; and R_{b} is D, halogen, OR, SR, N(R)R, —C(O)R, —C(O)OR, RC(O)O—, or S(O)ₙR; and
R is independently H, C1-C3 alkyl, C2-C3 alkenyl, C2-C3 alkynyl, or C5-C6 cycloalkyl.

10. The aryl hydrocarbon receptor modulator according to claim 1, wherein the aryl hydrocarbon receptor modulator is the following compounds:

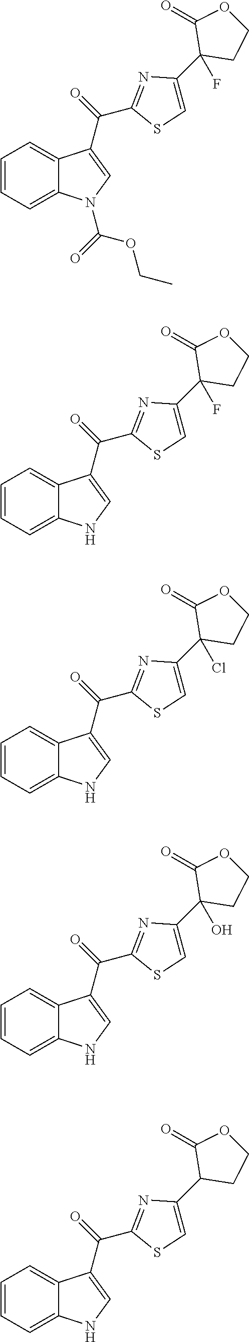 [SMILES: CCOC(=O)n1cc(C(=O)c2nc(C3(F)CCOC3=O)cs2)c2ccccc21 ; O=C(c1nc(C2(F)CCOC2=O)cs1)c1c[nH]c2ccccc12 ; O=C(c1nc(C2(Cl)CCOC2=O)cs1)c1c[nH]c2ccccc12 ; O=C(c1nc(C2(O)CCOC2=O)cs1)c1c[nH]c2ccccc12 ; O=C(c1nc(C2CCOC2=O)cs1)c1c[nH]c2ccccc12]

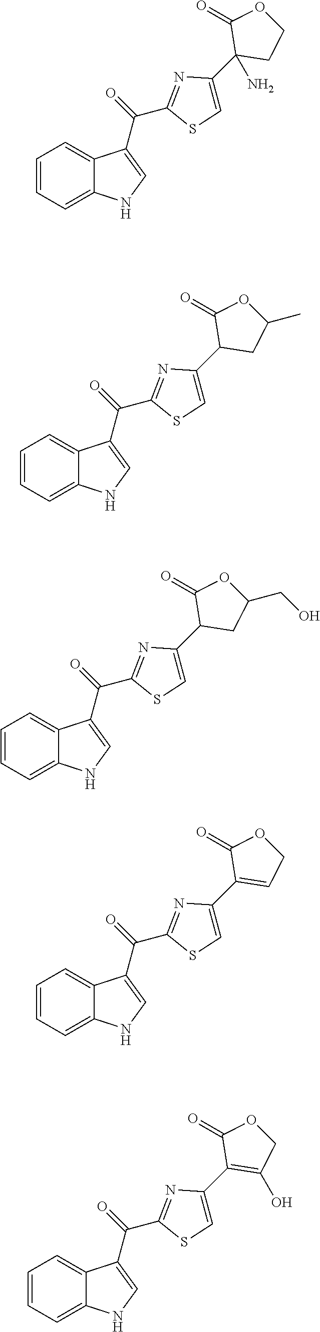 [SMILES: NC1(c2csc(C(=O)c3c[nH]c4ccccc34)n2)CCOC1=O ; CC1CC(c2csc(C(=O)c3c[nH]c4ccccc34)n2)C(=O)O1 ; O=C(c1nc(C2CC(CO)OC2=O)cs1)c1c[nH]c2ccccc12 ; O=C1OCC=C1c1csc(C(=O)c2c[nH]c3ccccc23)n1 ; O=C1OCC(O)=C1c1csc(C(=O)c2c[nH]c3ccccc23)n1]

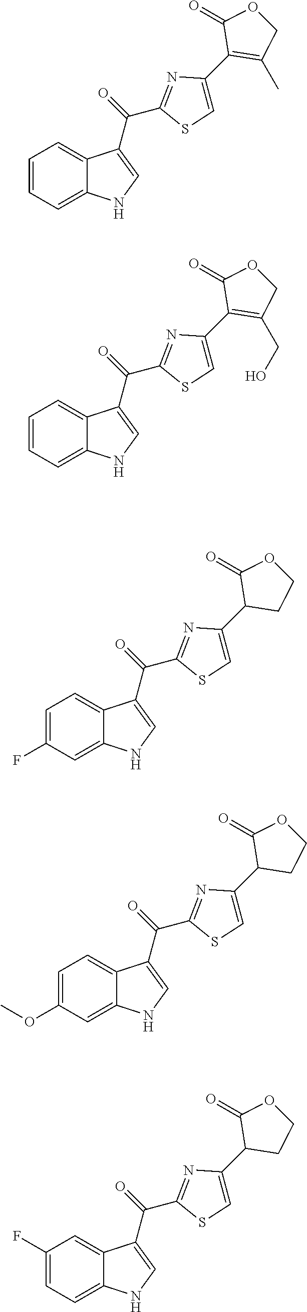

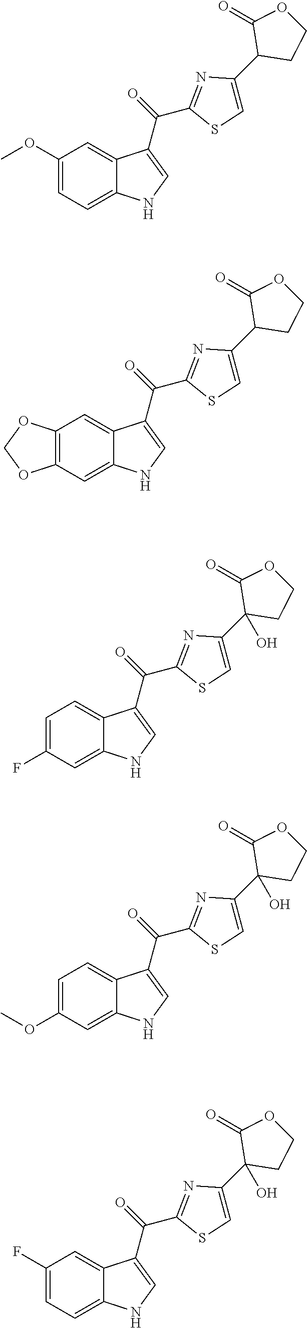 [SMILES: COc1ccc2[nH]cc(C(=O)c3nc(C4CCOC4=O)cs3)c2c1 ; O=C(c1nc(C2CCOC2=O)cs1)c1c[nH]c2cc3c(cc12)OCO3 ; O=C(c1nc(C2(O)CCOC2=O)cs1)c1c[nH]c2cc(F)ccc12 ; COc1ccc2c(C(=O)c3nc(C4(O)CCOC4=O)cs3)c[nH]c2c1 ; O=C(c1nc(C2(O)CCOC2=O)cs1)c1c[nH]c2ccc(F)cc12]

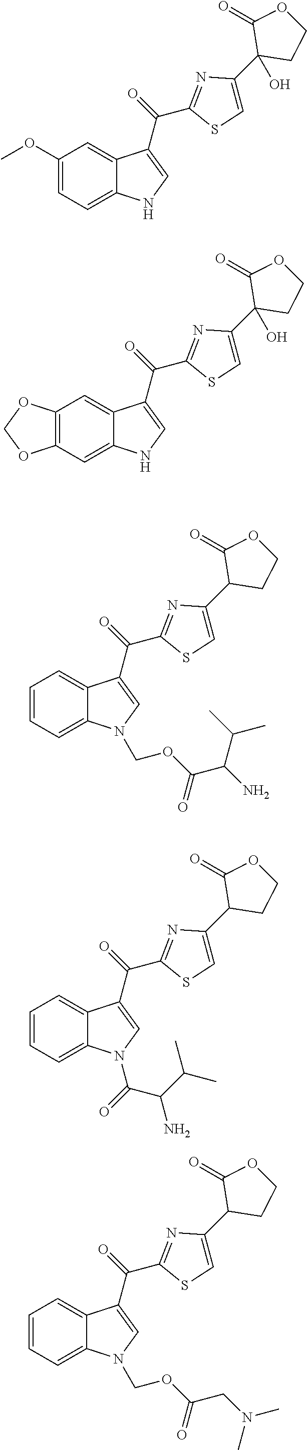

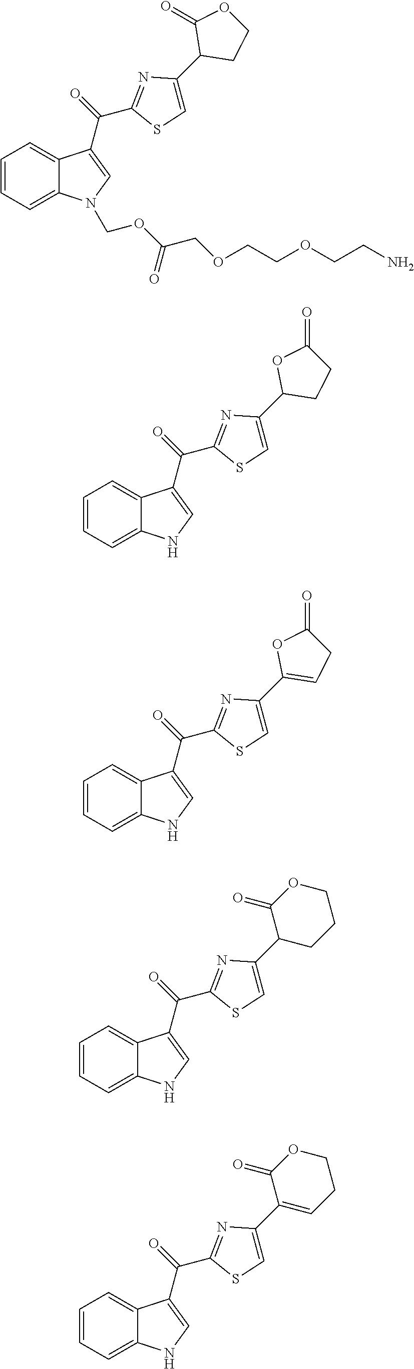 [SMILES: NCCOCCOCC(=O)OCn1cc(C(=O)c2nc(C3CCOC3=O)cs2)c2ccccc21 ; O=C1CCC(c2csc(C(=O)c3c[nH]c4ccccc34)n2)O1 ; O=C1CC=C(c2csc(C(=O)c3c[nH]c4ccccc34)n2)O1 ; O=C(c1nc(C2CCCOC2=O)cs1)c1c[nH]c2ccccc12 ; O=C1OCCC=C1c1csc(C(=O)c2c[nH]c3ccccc23)n1]

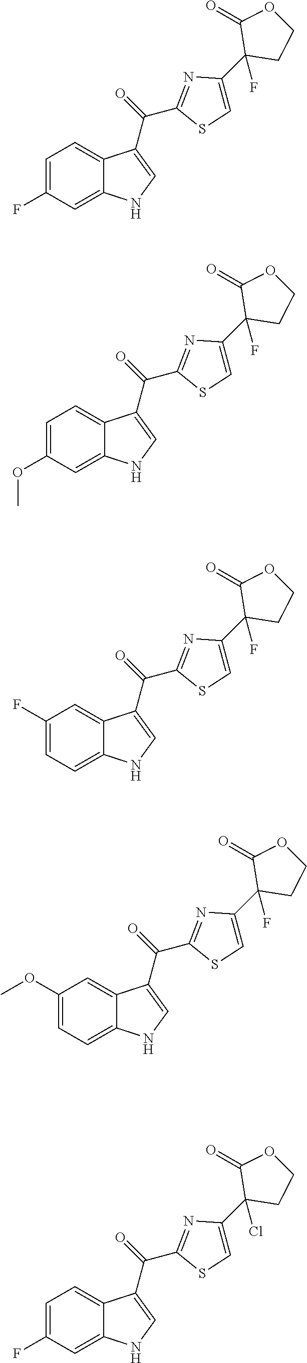 [SMILES: O=C(c1nc(C2(F)CCOC2=O)cs1)c1c[nH]c2cc(F)ccc12 ; COc1ccc2c(C(=O)c3nc(C4(F)CCOC4=O)cs3)c[nH]c2c1 ; O=C(c1nc(C2(F)CCOC2=O)cs1)c1c[nH]c2ccc(F)cc12 ; COc1ccc2[nH]cc(C(=O)c3nc(C4(F)CCOC4=O)cs3)c2c1 ; O=C(c1nc(C2(Cl)CCOC2=O)cs1)c1c[nH]c2cc(F)ccc12]

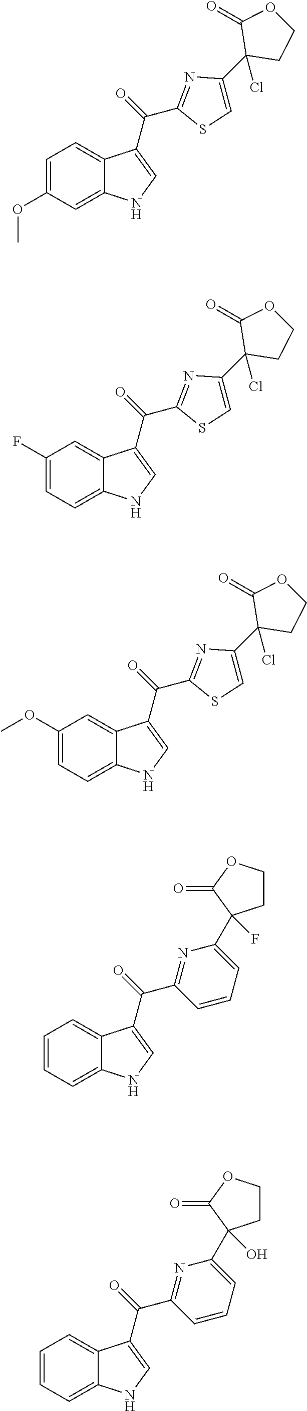 [SMILES: COc1ccc2c(C(=O)c3nc(C4(Cl)CCOC4=O)cs3)c[nH]c2c1 ; O=C(c1nc(C2(Cl)CCOC2=O)cs1)c1c[nH]c2ccc(F)cc12 ; COc1ccc2[nH]cc(C(=O)c3nc(C4(Cl)CCOC4=O)cs3)c2c1 ; O=C(c1cccc(C2(F)CCOC2=O)n1)c1c[nH]c2ccccc12 ; O=C(c1cccc(C2(O)CCOC2=O)n1)c1c[nH]c2ccccc12]

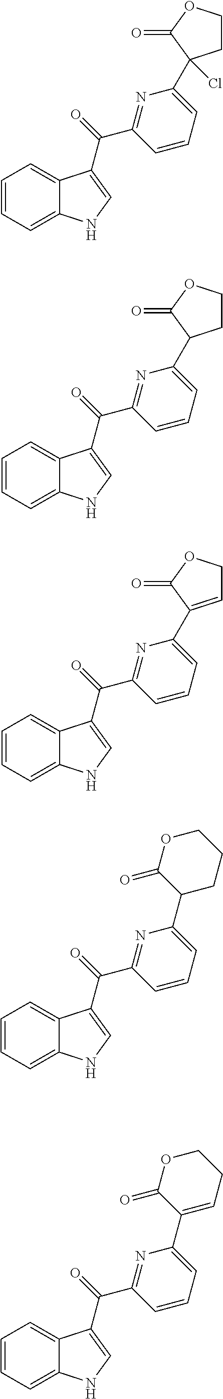 [SMILES: O=C(c1cccc(C2(Cl)CCOC2=O)n1)c1c[nH]c2ccccc12 ; O=C(c1cccc(C2CCOC2=O)n1)c1c[nH]c2ccccc12 ; O=C1OCC=C1c1cccc(C(=O)c2c[nH]c3ccccc23)n1 ; O=C(c1cccc(C2CCCOC2=O)n1)c1c[nH]c2ccccc12 ; O=C1OCCC=C1c1cccc(C(=O)c2c[nH]c3ccccc23)n1]

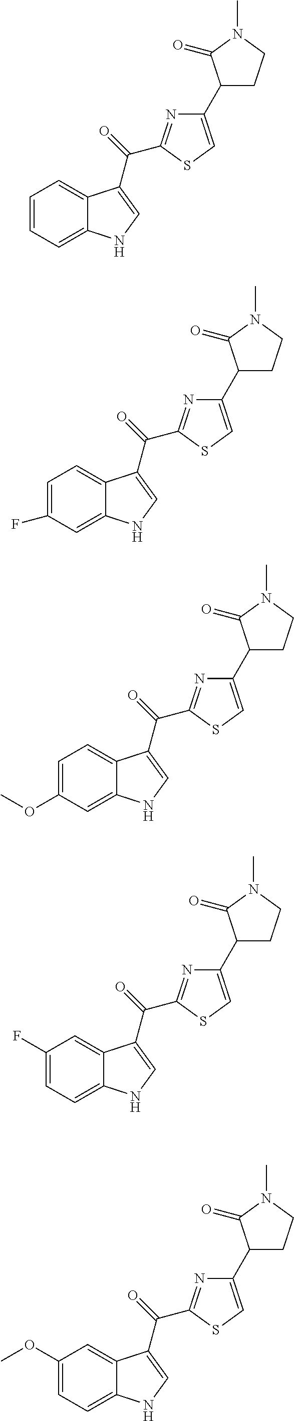 [SMILES: CN1CCC(c2csc(C(=O)c3c[nH]c4ccccc34)n2)C1=O ; CN1CCC(c2csc(C(=O)c3c[nH]c4cc(F)ccc34)n2)C1=O ; COc1ccc2c(C(=O)c3nc(C4CCN(C)C4=O)cs3)c[nH]c2c1 ; CN1CCC(c2csc(C(=O)c3c[nH]c4ccc(F)cc34)n2)C1=O ; COc1ccc2[nH]cc(C(=O)c3nc(C4CCN(C)C4=O)cs3)c2c1]

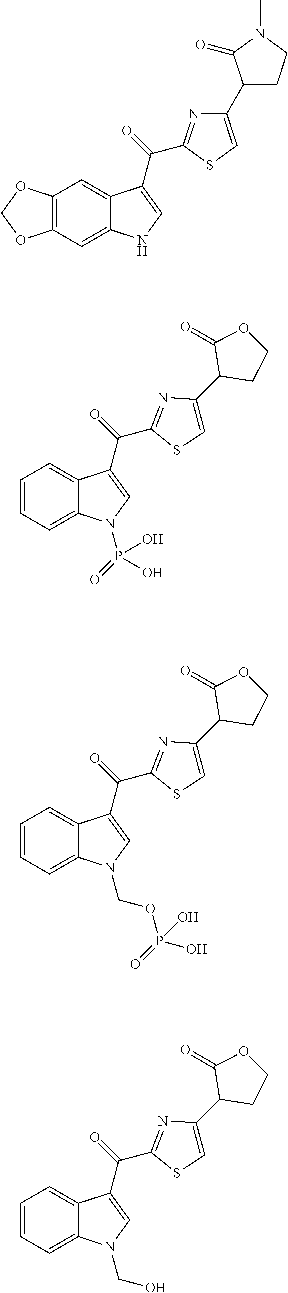 [SMILES: CN1CCC(c2csc(C(=O)c3c[nH]c4cc5c(cc34)OCO5)n2)C1=O ; O=C(c1nc(C2CCOC2=O)cs1)c1cn(P(=O)(O)O)c2ccccc12 ; O=C(c1nc(C2CCOC2=O)cs1)c1cn(COP(=O)(O)O)c2ccccc12 ; O=C(c1nc(C2CCOC2=O)cs1)c1cn(CO)c2ccccc12]

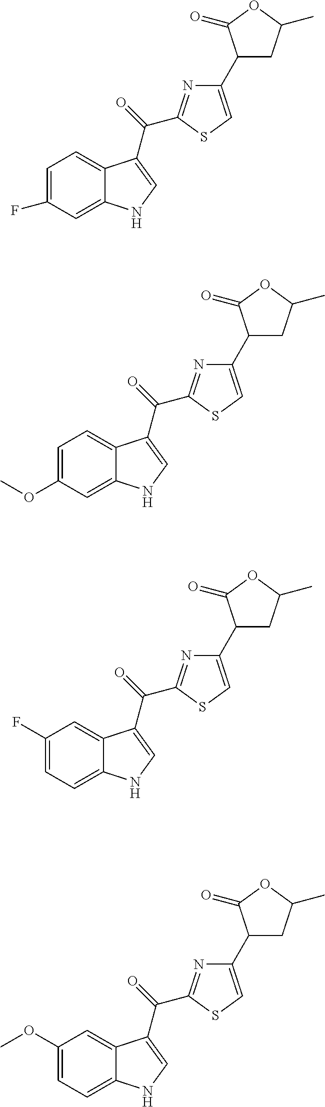

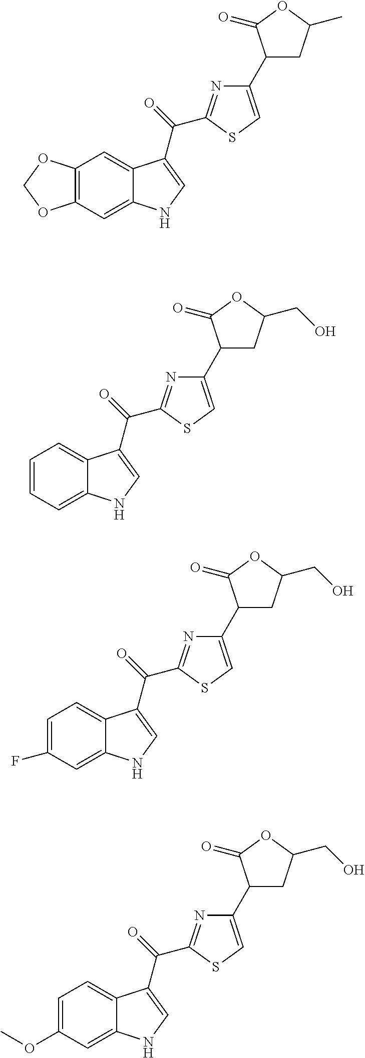

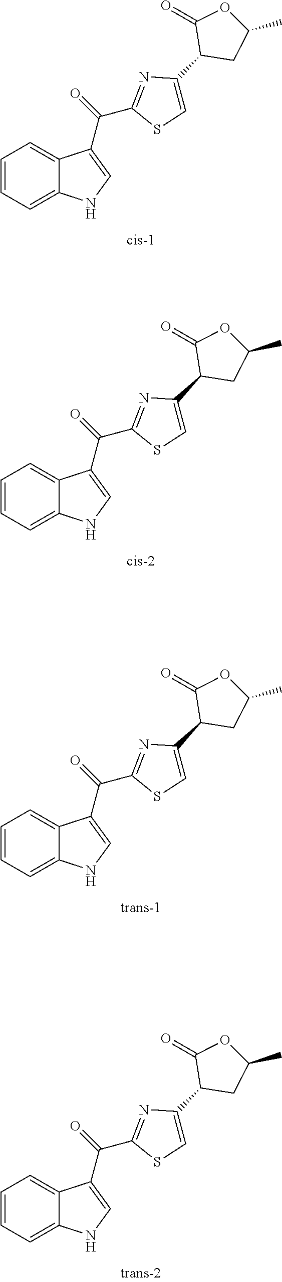 [SMILES: C[C@@H]1C[C@H](c2csc(C(=O)c3c[nH]c4ccccc34)n2)C(=O)O1 ; C[C@H]1C[C@@H](c2csc(C(=O)c3c[nH]c4ccccc34)n2)C(=O)O1 ; C[C@@H]1C[C@@H](c2csc(C(=O)c3c[nH]c4ccccc34)n2)C(=O)O1 ; C[C@H]1C[C@H](c2csc(C(=O)c3c[nH]c4ccccc34)n2)C(=O)O1]

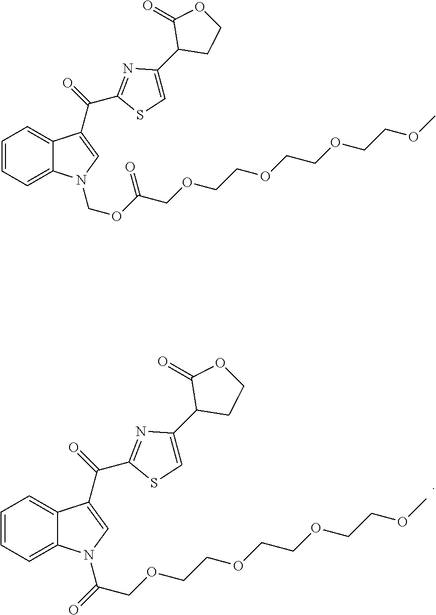 [SMILES: COCCOCCOCCOCC(=O)OCn1cc(C(=O)c2nc(C3CCOC3=O)cs2)c2ccccc21 ; COCCOCCOCCOCC(=O)n1cc(C(=O)c2nc(C3CCOC3=O)cs2)c2ccccc21]

11. A salt, a prodrug, a hydrate, a solvate, or a deuterated derivative further substituted with deuterium of the aryl hydrocarbon receptor modulator according to claim 1.
